(19) United States
(12) Patent Application Publication
(10) Pub. No.: US 2022/0233560 A1
(43) Pub. Date: Jul. 28, 2022

(54) OLIGOSACCHARIDE COMPOSITIONS AND METHODS OF USE

(71) Applicant: Kaleido Biosciences, Inc., Lexington, MA (US)

(72) Inventors: Tatyana Yatsunenko, Lexington, MA (US); Jonathan Lawrence, Lexington, MA (US); Madeline Rosini, Lexington, MA (US); Max Hecht, Lexington, MA (US); Christopher Matthew Liu, Somerville, MA (US)

(73) Assignee: Kaleido Biosciences, Inc., Lexington, MA (US)

(21) Appl. No.: 17/609,234

(22) PCT Filed: May 8, 2020
(86) PCT No.: PCT/US2020/032240

§ 371 Date: Nov. 5, 2021

Related U.S. Application Data
(60) Provisional application No. 62/910,179, filed on Oct. 3, 2019.
(60) Provisional application No. 62/845,305, filed on May 8, 2019.

(51) Int. Cl. A61K 31/702; A61P 1/00; A61P 31/04; C07H 3/06; C07H 1/00; A61P 31/10; A61K 35/741; A61K 45/06

(52) U.S. Cl. CPC A61K 31/702; A61P 1/00; A61P 31/04; C07H 3/06; A61K 2035/115; A61P 31/10; A61K 35/741; A61K 45/06; C07H 1/00

(57) ABSTRACT

Aspects of the disclosure relate to oligosaccharide compositions and methods of making the same. Also provided are methods of using oligosaccharide compositions as microbiome metabolic therapies for reducing pathogen levels and/or abundance and for the treatment of related diseases.

### RELATED APPLICATIONS

This application claims the benefit of the filing date of U.S. Provisional Application No. 62/845,305, entitled “OLIGOSACCHARIDE COMPOSITIONS AND METHODS OF USE THEREOF”, filed May 8, 2019; and U.S. Provisional Application No. 62/910,179, entitled “OLIGOSACCHARIDE COMPOSITIONS AND METHODS OF USE THEREOF”, filed Oct. 3, 2019; the contents of each of which are incorporated herein by reference in their entirety.

### FIELD OF THE INVENTION

The present disclosure relates to oligosaccharide compositions and uses thereof.

### BACKGROUND OF INVENTION

Hospital-acquired infection by antibiotic-resistant bacteria presents a global health crisis. Infections with carbapenem-resistant Enterobacteriaceae (CRE) and vancomycin-resistant Enterococci (VRE) can result in a 50% mortality rate in compromised hosts. A major risk factor for clinical infection is intestinal colonization with CRE or VRE.

Colonization may be associated with transmission as well as increased risk of infection. CRE and vancomycin-resistant Enterococcus (VRE) colonization are associated with increased risk of infection. The relative risk of infection is higher in VRE and CRE colonized patients compared to non-colonized patients. There is also a much higher risk of bloodstream infection (BSI) with colonization by extended-spectrum beta-lactamase (ESBL) producing organisms. Furthermore, colonization is highly associated with spread of hospital acquired infections (HAI). CRE, VRE and C. difficile infections are associated with being acquired after receiving healthcare (e.g., central line-associated bloodstream infections (CLABSI), catheter-associated urinary tract Infection (CAUTI), and C. difficile infections (CDI)). The level of these pathogens in the gut generally correlates with the risk of resistant infections. Thus, there is a need for effective treatments that decolonize patients from these organisms.

### SUMMARY OF INVENTION

According to some aspects, provided herein are microbiome metabolic therapies utilizing oligosaccharide compositions that are useful for driving functional outputs of the gut microbiome organ, e.g., to treat disease. Some aspects of the disclosure relate to a recognition that commensal microbes offer protection from pathogen infection and that in immunocompromised hosts or with antibiotic treatment, the protective properties of the microbial community are compromised, leaving the gut susceptible to pathogen colonization. In some embodiments, microbiome metabolic therapies utilizing oligosaccharide compositions are particularly effective for reducing the acquisition of, colonization of, or reducing the reservoir of a pathogen (e.g., a drug or antibiotic resistant pathogen, or an MDR pathogen) in a subject, e.g., by modulating the abundance (e.g., relative abundance or absolute abundance) of commensal microbial populations. An exemplary mechanism for the reduction of pathogens and increase of commensal bacteria is shown in FIG. 1. For example, in some embodiments microbiome metabolic therapies utilizing oligosaccharide compositions disclosed herein are useful for treating a subject having or at risk of developing an infection (e.g., a gastrointestinal infection, or another infection, e.g. blood stream infection or urinary tract infection, or infections of other organs, e.g. respiratory system, cardiovascular system, etc.) caused by a pathogen (e.g., a bacterial or fungal pathogen or pathobiont (e.g., an opportunistic pathogen that is a symbiotic organism capable of causing disease only when certain genetic and/or environmental conditions are present), including a pathogen resistant to most or even all available antibiotics) such as carbapenem-resistant Enterobacteriaceae (CRE), vancomycin-resistant Enterococcus (VRE) and extended-spectrum beta lactamase-producing Enterobacteriaceae (ESBLE). These are considered urgent (CRE) and serious threats (VRE, ESBLE), respectively, by the Centers for Disease Control.

Provided herein are selected synthetic oligosaccharide compositions that affect the structure (e.g., composition) and/or function (e.g. metabolic activity) of the gut microbiota. In some embodiments, the selected oligosaccharide compositions confer beneficial health effects on a subject. In some embodiments, the selected oligosaccharide compositions described herein reduce the abundance (e.g., relative abundance or absolute abundance) of pathogens or pathobionts (e.g., in the gastrointestinal tract), e.g., when compared to a baseline (e.g., untreated (population of) subject(s), or a subject prior to treatment). In some embodiments, the selected oligosaccharide compositions described herein promote growth of commensal bacteria over growth of pathogens or pathobionts (e.g., in the gastrointestinal tract, e.g., the intestines, e.g., the large intestine or colon). In some embodiments, subjects achieve decolonization with MDR pathogens (e.g., vancomycin-resistant Enterococcus (VRE), extended-spectrum beta lactamase-producing Enterobacteriaceae (ESBLE), and carbapenem-resistant Enterobacteriaceae (CRE), e.g., levels of these bacteria are near to or fall below detectable levels. The reduction in the abundance (e.g., relative abundance or absolute abundance) of a pathogen or pathobiont, may be determined, e.g., by subjecting a sample (e.g., a stool sample) from a subject to nucleic acid sequencing (e.g., whole genome sequencing) and other assays (e.g., colony-forming units (cfu)/g feces by culture). In some embodiments, the selected oligosaccharide compositions described herein promote an increase in alpha-diversity (e.g. an increase in bacterial taxa diversity, e.g., as determined by measuring Shannon diversity, e.g. by nucleic acid sequencing). In some embodiments, the selected oligosaccharide compositions described herein promote richness of the bacterial community. In some embodiments, the selected oligosaccharide compositions described herein reduce inflammation, e.g. inflammation associated with pathogens or pathobionts or other bacteria. The reduction may be determined by measuring one or more markers of inflammation, e.g. IFN-γ, IL-1β, IL-2, IL-4, IL-6, IL-8, IL-10, IL-12p70, IL-13, and TNF-α. The markers can be determined, e.g., from stool or blood samples. In some embodiments, the selected oligosaccharide compositions described herein reduce infections (e.g., the rate of infections), including secondary or opportunistic infections (e.g., hospital acquired infections (HAI)), including, e.g., central line-associated bloodstream infections (CLABSI), catheter-associated urinary tract Infection (CAUTI), and C. difficile infections (CDI)). In some embodiments, the selected oligosaccharide compositions described herein reduce the rate of hospitalizations, e.g., due to or caused by infections. In some embodiments, the selected oligosaccharide compositions described herein shorten the time period of hospitalization required, e.g., to treat or resolve the infections.

In some aspects, the disclosure provides an oligosaccharide composition comprising a plurality of oligosaccharides selected from Formula (I), Formula (II), and Formula (III):

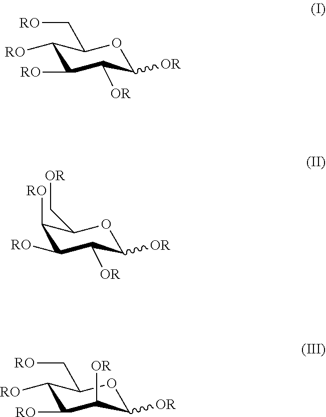

wherein each R independently is selected from hydrogen, and Formulae (Ia), (Ib), (Ic), (Id), (IIa), (IIb), (IIc), (IId), (IIIa), (IIIb), (IIIc), (IIId):

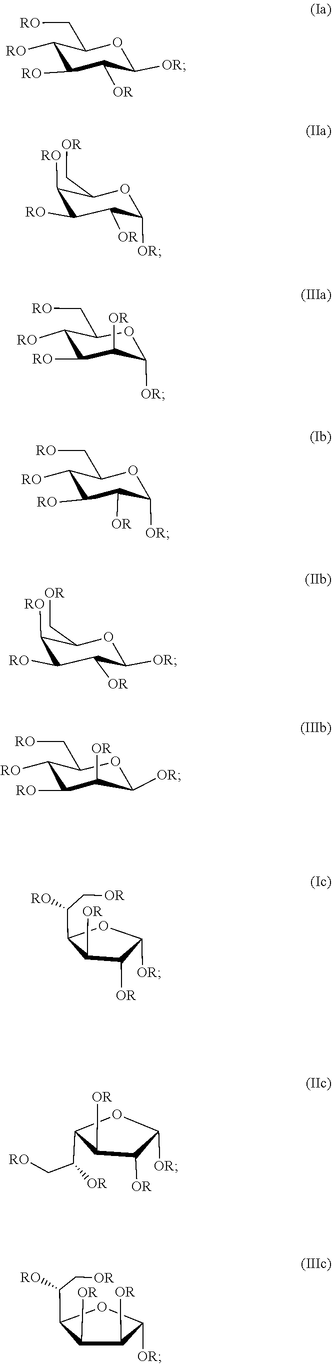

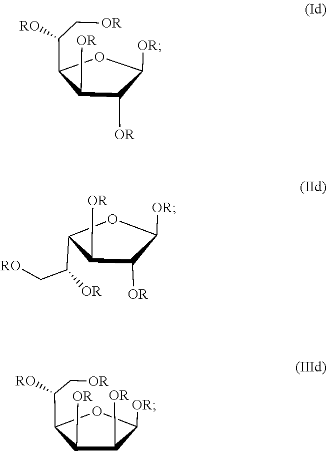

wherein each R independently is as defined above;
wherein the oligosaccharide composition is produced by a process comprising:

(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and

(b) promoting acid catalyzed oligosaccharide formation in the reaction mixture by transferring sufficient heat to the reaction mixture to maintain the reaction mixture at its boiling point.

In some embodiments, step (b) comprises promoting acid catalyzed oligosaccharide formation in the reaction mixture by transferring sufficient heat to the reaction mixture to maintain the reaction mixture at its boiling point until the weight percent of total monomer content in the oligosaccharide composition is in a range of 2% to 20%, wherein the total monomer content comprises dextrose monomer, galactose monomer, and/or mannose monomer.

In some aspects, the disclosure provides an oligosaccharide composition comprising a plurality of oligosaccharides selected from Formula (I), Formula (II), and Formula (III):

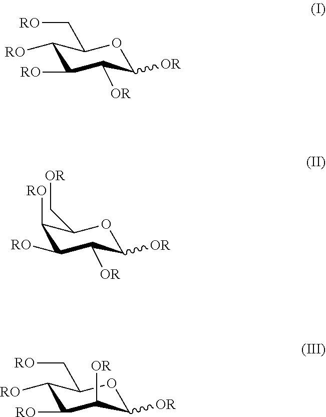

wherein each R independently is selected from hydrogen, and Formulae (IIa), (IIb), (IIc), (IId), (IIIa), (IIIb), (IIIc), (IIId):

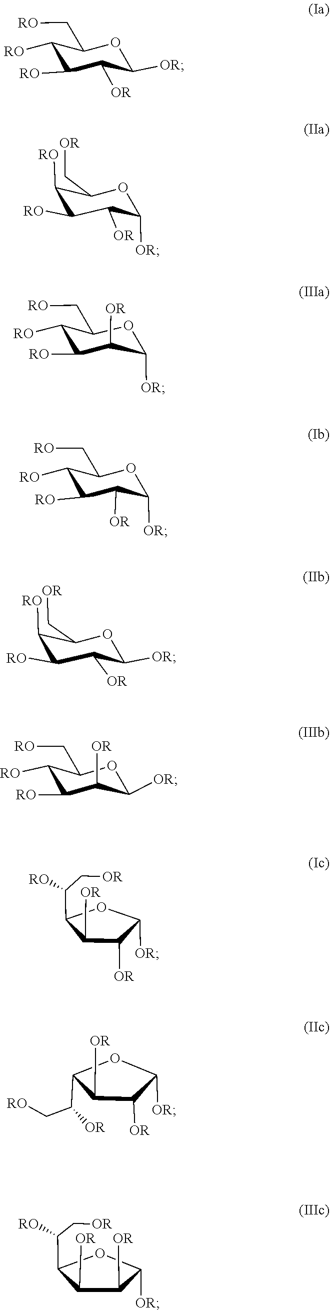

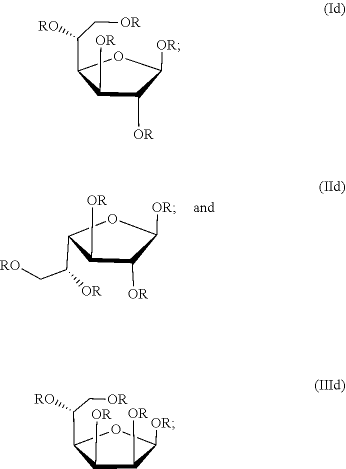

wherein each R independently is as defined above;
wherein the oligosaccharide composition is produced by a process comprising:

(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and

(b) maintaining the reaction mixture at its boiling point, at a pressure in the range of 0.5-1.5 atm, under conditions that promote acid catalyzed oligosaccharide formation, until the weight percent of total monomer content in the oligosaccharide composition is in a range of 2% to 20%, wherein the total monomer content comprises dextrose monomer, galactose monomer, and/or mannose monomer.

In some embodiments, step (b) comprises loading the preparation with an acid catalyst comprising positively charged hydrogen ions, in an amount such that the molar ratio of positively charged hydrogen ions to total dextrose monomer, galactose monomer, and mannose monomer content is in an appropriate range.

In some embodiments, steps (a) and (b) occur simultaneously.

In some embodiments, step (a) comprises heating the reaction mixture under agitation conditions to a temperature in a range of 100° C. to 160° C. In some embodiments, step (a) comprises heating the reaction mixture under agitation conditions to a temperature in a range of 135° C. to 145° C. In some embodiments, step (a) comprises heating the reaction mixture under agitation conditions at a temperature in a range of 100° C. to 160° C. In some embodiments, step (a) comprises heating the reaction mixture under agitation conditions at a temperature in a range of 135° C. to 145° C. In some embodiments, step (a) comprises gradually increasing the temperature (e.g., from room temperature) to about 140° C., under suitable conditions to achieve homogeneity and uniform heat transfer.

In some embodiments, step (b) comprises maintaining the reaction mixture at atmospheric pressure or under vacuum, at a temperature in a range of 135° C. to 145° C., under conditions that promote acid catalyzed oligosaccharide composition formation, until the weight percent of dextrose monomer, galactose monomer, and mannose monomer in the oligosaccharide composition is in a range of 4-14. In some embodiments, step (b) comprises gradually increasing the temperature (e.g., from room temperature) to about 140° C., under suitable conditions to achieve homogeneity and uniform heat transfer.

In some embodiments, said heating comprises melting the preparation and/or heating the preparation under suitable conditions to achieve homogeneity and uniform heat transfer. In some embodiments, the acid catalyst is a soluble catalyst. In some embodiments, the soluble catalyst is an organic acid, optionally a weak organic acid. In some embodiments, the acid catalyst is citric acid, acetic acid, or propionic acid. In some embodiments, the acid catalyst is a strong acid cation exchange resin having one or more physical and chemical properties according to Table 1 and/or wherein the catalyst comprises >3.0 mmol/g sulfonic acid moieties and <1.0 mmol/gram cationic moieties. In some embodiments, the catalyst has a nominal moisture content of 45-50 weight percent. In some embodiments, the catalyst has some or all of the properties shown in Table 1.

TABLE 1
Non-Limiting Example of Strong Acid
Cation Exchange Resin Properties

| Physical Form | | Amber translucent spherical |
|---|---|---|
| | | beads |
| Matrix | | Styrene-DVB, gel |
| Function group | | Sulfonic acid |
| Ionic form as shipped | | H⁺ form |
| Total volume capacity, min. | eq/L | 1.8 |
| | kgr/ft³ as | 39.3 |
| | CaCO₃ | |
| Moisture retention capacity | % | 50-56 |
| Particle size | | |
| Uniformity coefficient, max. | | 1.1 |
| Harmonic mean diameter | μm | 600 ± 50 |
| Whole uncracked beads | % | 95-100 |
| Total swelling (Na⁺ → H⁺) | % | 8 |
| Particle density | g/mL | 1.2 |
| Shipping density | g/L | 800 |
| | lbs/ft³ | 50 |

In some embodiments, the oligosaccharide composition further comprises water at a level below that which is necessary for microbial growth upon storage at room temperature. Methods for controlling moisture levels to address microbial growth are described in Ergun, R. et al, “Moisture and Shelf Life in Sugar Confections, Critical Reviews in Food Science and Nutrition”, 2010, 50:2, 162-192; and NIROOMAND, F. et al. “Fate of Bacterial Pathogens and Indicator Organisms in Liquid Sweeteners” Journal of Food Protection, Vol. 61, No. 3, 1998, Pages 295-299; the contents of each are incorporated in their entirety.

In some embodiments, step (b) further comprises removing water from the reaction mixture by evaporation. In some embodiments, step (b) further comprises maintaining the reaction mixture at 93-94 weight percent dissolved solids.

In some embodiments, the process further comprises: (c) quenching the reaction mixture, for example, using water, while bringing the temperature of the reaction mixture to a temperature in the range of 55° C. to 95° C. (e.g., 85° C., 90° C.); and optionally, (d) separating oligosaccharide composition from the acid catalyst.

In some embodiments, in step (c) the water is deionized water. In some embodiments, in (c) the water has a temperature of about 95° C. In some embodiments, in (c) the water is added to the reaction mixture under conditions sufficient to avoid solidifying the mixture.

In some embodiments, in step (d) said separating comprises removing the catalyst by filtration. In some embodiments, (d) comprises cooling the reaction mixture to below about 85° C. before filtering.

In some embodiments, the process further comprises: (e) diluting the oligosaccharide composition of (d) with water to a concentration of about 45-55 weight percent; (f) passing the diluted composition through a cationic exchange resin; (g) passing the diluted composition through a decolorizing polymer resin; and/or (h) passing the diluted composition through an anionic exchange resin; wherein each of (f), (g), and (h) can be performed one or more times in any order.

In some embodiments, the process further comprises diluting the oligosaccharide composition of (d) with water to a concentration of about 35-55 weight percent and passing the diluted composition through a 45 μm filter.

In some embodiments, wherein the composition comprises water at a level below that which is necessary for microbial growth upon storage at room temperature.

In some embodiments, the composition comprises water in a range of 45-55 weight percent.

In some embodiments, the composition has a MWw (g/mol) in a range of 1905-2290. In some embodiments, the composition has a MWw (g/mol) in a range of 1740-2407. In some embodiments, the composition has a MWw (g/mol) in a range of 1863-2268. In some embodiments, the composition has a MWw (g/mol) in a range of 1700-2295. In some embodiments, the composition has a MWn (g/mol) in a range of 1033-1184. In some embodiments, the composition has a MWn (g/mol) in a range of 975-1155. In some embodiments, the composition has a MWn (g/mol) in a range of 984-1106. In some embodiments, the composition has a MWn (g/mol) in a range of 938-1120.

In some embodiments, a solution comprising the oligosaccharide composition has a pH in a range of 2.50-7.00, optionally 2.50-3.50.

In some embodiments, the composition comprises oligomers having two or more repeat units (DP2+) in a range of 86-96 weight percent. In some embodiments, the composition comprises oligomers having two or more repeat units (DP2+) in a range of 81-100 weight percent. In some embodiments, the composition comprises oligomers having at least three linked monomer units (DP3+) in a range of 85-90 weight percent.

3In some embodiments, the composition further comprises: 0.18-0.51% w/w levoglucosan, 0.01-0.05% w/w lactic acid, and/or 0.04-0.07% w/w formic acid.

In some embodiments, the composition further comprises: 0.40-0.53% w/w levoglucosan, 0.01-0.02% w/w lactic acid, 0.01-0.04% w/w formic acid, and/or 0.01-0.04% w/w citric acid.

In some aspects, the disclosure provides an oligosaccharide composition comprising a plurality of oligosaccharides that are minimally digestible in humans, the composition being characterized by a multiplicity-edited gradient-enhanced ¹H-¹³C heteronuclear single quantum correlation (HSQC) NMR spectrum comprising signals 5, 6, 7, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, the oligosaccharide composition is characterized by a multiplicity-edited gradient-enhanced ¹H-¹³C heteronuclear single quantum correlation (HSQC) NMR spectrum comprising signals 5, 6, 7, 10, 14, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, the oligosaccharide composition is characterized by a multiplicity-edited gradient-enhanced ¹H-¹³C heteronuclear single quantum correlation (HSQC) NMR spectrum comprising signals 5, 6, 7, and 10-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, the oligosaccharide composition is characterized by a multiplicity-edited gradient-enhanced ¹H-¹³C heteronuclear single quantum correlation (HSQC) NMR spectrum comprising signals 1-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some aspects, the disclosure provides an oligosaccharide composition comprising a plurality of oligosaccharides that are minimally digestible in humans, the composition being characterized by a multiplicity-edited gradient-enhanced ¹H-¹³C heteronuclear single quantum correlation (HSQC) NMR spectrum comprising signals 5, 6, 7, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, the oligosaccharide composition is characterized by a multiplicity-edited gradient-enhanced ¹H-¹³C heteronuclear single quantum correlation (HSQC) NMR spectrum comprising signals 5, 6, 7, 10, 14, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, the oligosaccharide composition is characterized by a multiplicity-edited gradient-enhanced ¹H-¹³C heteronuclear single quantum correlation (HSQC) NMR spectrum comprising signals 5, 6, 7, and 10-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, the oligosaccharide composition is characterized by a multiplicity-edited gradient-enhanced ¹H-¹³C heteronuclear single quantum correlation (HSQC) NMR spectrum comprising signals 1-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, any one of signals 1-15 are further characterized by an ¹H integral region and a ¹³C integral region, defined as follows:

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | to |
| 1 | 3.68 | 3.61 | 3.75 | 63.42 | 62.64 | 64.20 |
| 2 | 3.75 | 3.72 | 3.78 | 66.06 | 65.50 | 66.62 |
| 3 | 3.97 | 3.94 | 4.00 | 66.15 | 65.81 | 66.49 |
| 4 | 3.96 | 3.94 | 3.98 | 69.28 | 69.04 | 69.52 |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 8 | 3.97 | 3.94 | 4.00 | 71.56 | 71.29 | 71.84 |
| 9 | 3.72 | 3.67 | 3.77 | 72.35 | 71.95 | 72.74 |
| 10 | 3.33 | 3.27 | 3.4 | 73.74 | 73.26 | 74.22 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |
| 14 | 4.5 | 4.47 | 4.54 | 103.29 | 102.87 | 103.70 |
| 15 | 4.44 | 4.41 | 4.46 | 103.86 | 103.56 | 104.15 |

In some aspects, the disclosure provides an oligosaccharide composition comprising a plurality of oligosaccharides that are minimally digestible in humans, each oligosaccharide comprising a plurality of monomer radicals;

the plurality of oligosaccharides comprising two or more types of monomer radicals selected from radicals (1)-(40):

(1) t-mannopyranose monoradicals, representing 3.0-4.1 mol % of monomer radicals in the plurality of oligosaccharides;

(2) t-glucopyranose monoradicals, representing 11.4-16.3 mol % of monomer radicals in the plurality of oligosaccharides;

(3) t-galactofuranose monoradicals, representing 1.3-7.8 mol % of monomer radicals in the plurality of oligosaccharides;

(4) t-glucofuranose monoradicals, representing 0.1-1.4 mol % of monomer radicals in the plurality of oligosaccharides;

(5) t-galactopyranose monoradicals, representing 8.3-12.5 mol % of monomer radicals in the plurality of oligosaccharides;

(6) 3-glucopyranose monoradicals, representing 3.0-4.9 mol % of monomer radicals in the plurality of oligosaccharides;

(7) 2-mannopyranose and/or 3-mannopyranose monoradicals, representing 1.2-1.9 mol % of monomer radicals in the plurality of oligosaccharides;

(8) 2-glucopyranose monoradicals, representing 2.4-3.2 mol % of monomer radicals in the plurality of oligosaccharides;

(9) 2-galactofuranose and/or 2-glucofuranose monoradicals, representing 0.9-2.3 mol % of monomer radicals in the plurality of oligosaccharides;

(10) 3-galactopyranose monoradicals, representing 2.9-3.9 mol % of monomer radicals in the plurality of oligosaccharides;

(11) 4-mannopyranose and/or 5-mannofuranose and/or 3-galactofuranose monoradicals, representing 1.7-2.9 mol % of monomer radicals in the plurality of oligosaccharides;

(12) 6-mannopyranose monoradicals, representing 2.0-2.9 mol % of monomer radicals in the plurality of oligosaccharides;

(13) 2-galactopyranose monoradicals, representing 1.8-2.7 mol % of monomer radicals in the plurality of oligosaccharides;

(14) 6-glucopyranose monoradicals, representing 7.6-10.8 mol % of monomer radicals in the plurality of oligosaccharides;

(15) 4-galactopyranose and/or 5-galactofuranose monoradicals, representing 2.6-3.8 mol % of monomer radicals in the plurality of oligosaccharides;

(16) 4-glucopyranose and/or 5-glucofuranose and/or 6-mannofuranose monoradicals, representing 3.0-4.5 mol % of monomer radicals in the plurality of oligosaccharides;

(17) 6-glucofuranose monoradicals, representing 0.1-1.6 mol % of monomer radicals in the plurality of oligosaccharides;

(18) 6-galactofuranose monoradicals, representing 1.4-5.0 mol % of monomer radicals in the plurality of oligosaccharides;

(19) 6-galactopyranose monoradicals, representing 5.8-9.1 mol % of monomer radicals in the plurality of oligosaccharides;

(20) 3,4-galactopyranose and/or 3,5-galactofuranose and/or 2,3-galactopyranose diradicals, representing 0.9-1.4 mol % of monomer radicals in the plurality of oligosaccharides;

(21) 3,4-glucopyranose and/or 3,5-glucofuranose diradicals, representing 0.1-1.1 mol % of monomer radicals in the plurality of oligosaccharides;

(22) 2,4-glucopyranose and/or 2,5-glucofuranose and/or 2,4-galactopyranose and/or 2,5-galactofuranose diradicals, representing 0.9-1.4 mol % of monomer radicals in the plurality of oligosaccharides;

(23) 4,6-mannopyranose and/or 5,6-mannofuranose diradicals, representing 0.5-0.7 mol % of monomer radicals in the plurality of oligosaccharides;

(24) 3,6-mannofuranose diradicals, representing 0.1 mol % of monomer radicals in the plurality of oligosaccharides;

(25) 3,6-glucopyranose diradicals, representing 1.4-2.8 mol % of monomer radicals in the plurality of oligosaccharides;

(26) 3,6-mannopyranose and/or 2,6-mannofuranose diradicals, representing 0.4-0.7 mol % of monomer radicals in the plurality of oligosaccharides;

(27) 2,6-mannopyranose diradicals, representing 0.3-0.5 mol % of monomer radicals in the plurality of oligosaccharides;

(28) 3,6-glucofuranose diradicals, representing 0.1-0.4 mol % of monomer radicals in the plurality of oligosaccharides;

(29) 2,6-glucopyranose and/or 4,6-glucopyranose and/or 5,6-glucofuranose diradicals, representing 1.1-3.6 mol % of monomer radicals in the plurality of oligosaccharides;

(30) 3,6-galactofuranose diradicals, representing 0.9-1.4 mol % of monomer radicals in the plurality of oligosaccharides;

(31) 4,6-galactopyranose and/or 5,6-galactofuranose diradicals, representing 2.1-2.9 mol % of monomer radicals in the plurality of oligosaccharides;

(32) 3,6-galactopyranose and/or 2,6-galactofuranose diradicals, representing 1.6-3.0 mol % of monomer radicals in the plurality of oligosaccharides;

(33) 2,6-galactopyranose diradicals, representing 0.7-1.6 mol % of monomer radicals in the plurality of oligosaccharides;

(34) 3,4,6-mannopyranose and/or 3,5,6-mannofuranose and/or 2,3,6-mannofuranose triradicals, representing 0.1-0.3 mol % of monomer radicals in the plurality of oligosaccharides;

(35) 3,4,6-galactopyranose and/or 3,5,6-galactofuranose and/or 2,3,6-galactofuranose triradicals, representing 0.5-1.1 mol % of monomer radicals in the plurality of oligosaccharides;

(36) 3,4,6-glucopyranose and/or 3,5,6-glucofuranose triradicals, representing 0.2-0.5 mol % of monomer radicals in the plurality of oligosaccharides;

(37) 2,3,6-mannopyranose and/or 2,4,6-mannopyranose and/or 2,5,6-mannofuranose triradicals, representing 0.1-0.5 mol % of monomer radicals in the plurality of oligosaccharides;

(38) 2,4,6-glucopyranose and/or 2,5,6-glucofuranose triradicals, representing 0.1-1.4 mol % of monomer radicals in the plurality of oligosaccharides;

(39) 2,3,6-galactopyranose and/or 2,4,6-galactopyranose and/or 2,5,6-galactofuranose triradicals, representing 0.4-0.9 mol % of monomer radicals in the plurality of oligosaccharides; and

(40) 2,3,6-glucopyranose triradicals, representing 0.1-0.5 mol % of monomer radicals in the plurality of oligosaccharides;

the oligosaccharide composition comprising at least one glucofuranose or glucopyranose radical, at least one mannofuranose or mannopyranose radical, and at least one galactofuranose or galactopyranose radical.

In some aspects, the disclosure provides an oligosaccharide composition comprising a plurality of oligosaccharides that are minimally digestible in humans, each oligosaccharide comprising a plurality of monomer radicals;

the plurality of oligosaccharides comprising two or more types of monomer radicals selected from radicals (1)-(43):

(1) t-mannopyranose monoradicals, representing 3.0-4.1 mol % of monomer radicals in the plurality of oligosaccharides;

(2) t-glucopyranose monoradicals, representing 13.6-17.6 mol % of monomer radicals in the plurality of oligosaccharides;

(3) t-galactofuranose monoradicals, representing 3.0-4.2 mol % of monomer radicals in the plurality of oligosaccharides;

(4) t-glucofuranose monoradicals, representing 0.1-0.7 mol % of monomer radicals in the plurality of oligosaccharides;

(5) t-galactopyranose monoradicals, representing 9.7-11.7 mol % of monomer radicals in the plurality of oligosaccharides;

(6) 3-glucopyranose monoradicals, representing 3.8-4.6 mol % of monomer radicals in the plurality of oligosaccharides;

(7) 2-mannopyranose and/or 3-mannopyranose monoradicals, representing 0.8-2.0 mol % of monomer radicals in the plurality of oligosaccharides;

(8) 2-glucopyranose monoradicals, representing 2.7-3.0 mol % of monomer radicals in the plurality of oligosaccharides;

(9) 2-galactofuranose and/or 2-glucofuranose monoradicals and/or 3-glucofuranose, representing 0.8-1.8 mol % of monomer radicals in the plurality of oligosaccharides;

(10) 3-galactopyranose monoradicals, representing 2.8-3.8 mol % of monomer radicals in the plurality of oligosaccharides;

(11) 3-galactofuranose monoradicals, representing 1.6-2.2 mol % of monomer radicals in the plurality of oligosaccharides;

(12) 6-mannopyranose monoradicals, representing 2.1-2.5 mol % of monomer radicals in the plurality of oligosaccharides;

(13) 2-galactopyranose monoradicals, representing 1.7-2.4 mol % of monomer radicals in the plurality of oligosaccharides;

(14) 6-glucopyranose monoradicals, representing 9.5-11.1 mol % of monomer radicals in the plurality of oligosaccharides;

(15) 4-galactopyranose and/or 5-galactofuranose monoradicals, representing 2.5-3.1 mol % of monomer radicals in the plurality of oligosaccharides;

(16) 4-glucopyranose and/or 5-glucofuranose and/or 6-mannofuranose monoradicals, representing 3.0-3.9 mol % of monomer radicals in the plurality of oligosaccharides;

(17) 2,3-galactofuranose diradicals, representing 0.1-0.4 mol % of monomer radicals in the plurality of oligosaccharides;

(18) 6-glucofuranose monoradicals, representing 0.1-0.8 mol % of monomer radicals in the plurality of oligosaccharides;

(19) 6-galactofuranose monoradicals, representing 2.3-2.7 mol % of monomer radicals in the plurality of oligosaccharides;

(20) 6-galactopyranose monoradicals, representing 7.1-8.8 mol % of monomer radicals in the plurality of oligosaccharides;

(21) 3,4-galactopyranose and/or 3,5-galactofuranose and/or 2,3-galactopyranose diradicals, representing 0.9-1.1 mol % of monomer radicals in the plurality of oligosaccharides;

(22) 3,4-glucopyranose and/or 3,5-glucofuranose diradicals, representing 0.5-0.8 mol % of monomer radicals in the plurality of oligosaccharides;

(23) 2,3-glucopyranose diradicals, representing 0.1-2.1 mol % of monomer radicals in the plurality of oligosaccharides;

(24) 2,4-mannopyranose and/or 2,5-mannofuranose diradicals, representing 0.1-0.9 mol % of monomer radicals in the plurality of oligosaccharides;

(25) 2,4-glucopyranose and/or 2,5-glucofuranose and/or 2,4-galactopyranose and/or 2,5-galactofuranose diradicals, representing 0.5-1.9 mol % of monomer radicals in the plurality of oligosaccharides;

(26) 4,6-mannopyranose and/or 5,6-mannofuranose diradicals, representing 0.4- 0.7 mol % of monomer radicals in the plurality of oligosaccharides;

(27) 3,6-glucopyranose diradicals, representing 2.0-2.9 mol % of monomer radicals in the plurality of oligosaccharides;

(28) 3,6-mannopyranose diradicals, representing 0.4-0.7 mol % of monomer radicals in the plurality of oligosaccharides;

(29) 2,6-mannopyranose diradicals, representing 0.4-0.5 mol % of monomer radicals in the plurality of oligosaccharides;

(30) 3,6-glucofuranose diradicals, representing 0.1-0.3 mol % of monomer radicals in the plurality of oligosaccharides;

(31) 2,6-glucopyranose and/or 4,6-glucopyranose and/or 5,6-glucofuranose diradicals, representing 1.7-2.6 mol % of monomer radicals in the plurality of oligosaccharides;

(32) 3,6-galactofuranose diradicals, representing 0.9-1.2 mol % of monomer radicals in the plurality of oligosaccharides;

(33) 4,6-galactopyranose and/or 5,6-galactofuranose diradicals, representing 2.1-2.9 mol % of monomer radicals in the plurality of oligosaccharides;

(34) 3,6-galactopyranose diradicals, representing 2.0-2.7 mol % of monomer radicals in the plurality of oligosaccharides;

(35) 2,6-galactopyranose diradicals, representing 1.0-1.5 mol % of monomer radicals in the plurality of oligosaccharides;

(36) 3,4,6-mannopyranose and/or 3,5,6-mannofuranose and/or 2,3,6-mannofuranose triradicals, representing 0.1 mol % of monomer radicals in the plurality of oligosaccharides;

(37) 3,4,6-galactopyranose and/or 3,5,6-galactofuranose and/or 2,3,6-galactofuranose triradicals, representing 0.5-1.0 mol % of monomer radicals in the plurality of oligosaccharides;

(38) 3,4,6-glucopyranose and/or 3,5,6-glucofuranose triradicals, representing 0.1-0.6 mol % of monomer radicals in the plurality of oligosaccharides;

(39) 2,3,6-mannopyranose triradicals, representing 0.1-0.3 mol % of monomer radicals in the plurality of oligosaccharides;

(40) 2,4,6-glucopyranose and/or 2,5,6-glucofuranose triradicals, representing 0.1-0.8 mol % of monomer radicals in the plurality of oligosaccharides;

(41) 2,3,6-galactopyranose and/or 2,4,6-galactopyranose and/or 2,5,6-galactofuranose triradicals, representing 0.1-1.3 mol % of monomer radicals in the plurality of oligosaccharides;

(42) 2,4,6-galactopyranose and/or 2,5,6-galactofuranose triradicals, representing 0.1-0.9 mol % of monomer radicals in the plurality of oligosaccharides;

(43) 2,3,6-glucopyranose triradicals, representing 0.1-0.7 mol % of monomer radicals in the plurality of oligosaccharides;

the oligosaccharide composition comprising at least one glucofuranose or glucopyranose radical, at least one mannofuranose or mannopyranose radical, and at least one galactofuranose or galactopyranose radical.

In some embodiments, the molar percentages of the monomer radicals are determined using a permethylation assay.

In some embodiments, the composition is substantially non-absorbable in a human.

In some aspects, the disclosure provides a method of reducing a ratio of pathogenic bacteria to commensal bacteria in the gastrointestinal tract of a human subject. In some embodiments, a method of reducing a ratio of pathogenic bacteria to commensal bacteria in the gastrointestinal tract of a human subject comprises administering to the gastrointestinal tract of the subject an effective amount of an oligosaccharide composition as described herein.

In some aspects, the disclosure provides a method of reducing the relative or absolute abundance of pathogens in the gastrointestinal tract of a human subject. In some embodiments, a method of reducing the relative or absolute abundance of pathogens in the gastrointestinal tract of a human subject comprises administering to the gastrointestinal tract of the subject an effective amount of an oligosaccharide composition as described herein.

In some embodiments, the oligosaccharide composition is administered in an amount effective to modulate (e.g. reduce or inhibit) colonization or to modulate (e.g. increase) decolonization of the pathogen in the gut (e.g., small intestine, large intestine and/or colon) of the human subject. In some embodiments, the oligosaccharide composition is administered in an amount effective to reduce or inhibit colonization (e.g., colonization by VRE, CRE, and/or ESBLE). In some embodiments, the oligosaccharide composition is administered in an amount effective to increase decolonization (e.g., decolonization by VRE, CRE, and/or ESBLE).

In some embodiments, a method reduces the abundance of pathogenic bacteria in the gastrointestinal tract, relative to a control (e.g., a control subject or baseline measurement).

In some embodiments, a method increases the abundance of commensal bacteria in the gastrointestinal tract, relative to a control (e.g., a control subject or baseline measurement).

In some embodiments, the reduction of the relative or absolute abundance of pathogens is determined by performing nucleic acid sequencing (e.g., 16S metagenomic sequencing) of a fecal sample collected from the subject. In some embodiments, the reduction of the relative or absolute abundance of pathogens is determined by: (i) performing 16S metagenomic sequencing of a fecal sample collected from the subject prior to administration of the oligosaccharide composition; (ii) performing 16S metagenomic sequencing of a fecal sample collected from the subject following administration of the oligosaccharide composition; and (iii) comparing the relative or absolute abundance of pathogens determined using the sequencing data provided in (ii) relative to the relative or absolute abundance of pathogens determined using the sequencing data provided in (i).

In some aspects, the disclosure provides a method of treating a subject for a pathogen infection. In some embodiments, a method of treating a subject for a pathogen infection comprises administering to the gastrointestinal tract of the subject an effective amount of an oligosaccharide composition as described herein, thereby treating the subject.

In some embodiments, a method of treating a subject for a pathogen infection comprises administering to the gastrointestinal tract of the subject an effective amount of an oligosaccharide composition, wherein the oligosaccharide composition has an average degree of polymerization of 5-20 and comprises a plurality of oligosaccharides selected from Formula (I), Formula (II), and Formula (III):

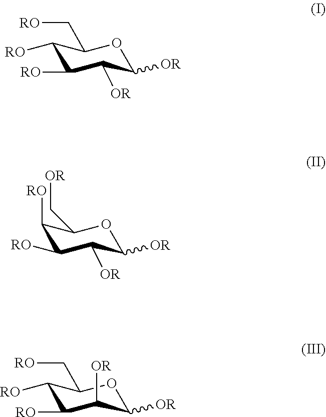

wherein each R independently is selected from hydrogen, and Formulae (Ia), (Ib), (Ic), (Id), (IIa), (IIb), (IIc), (IId), (IIIa), (IIIb), (IIIc), (IIId):

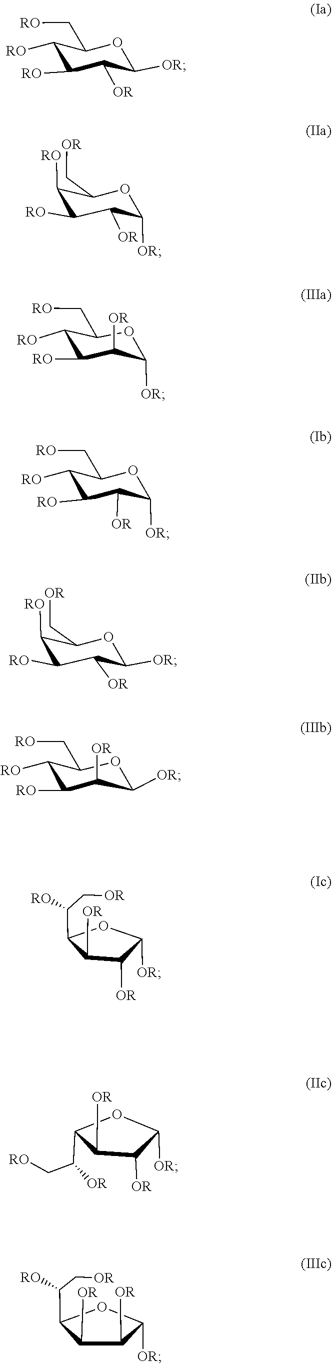

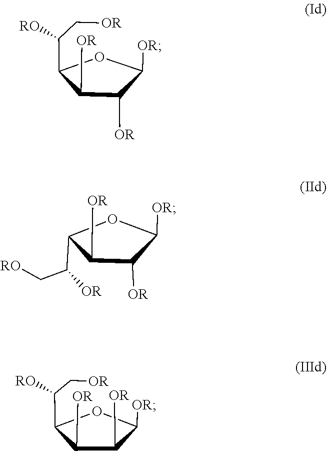

wherein each R independently is as defined above;

thereby treating the subject.

In some embodiments, a method reduces the rate of infection. In some embodiments, a method reduces the abundance of pathogen. In some embodiments, a method reduces the abundance of pathogen of infection by at least 5%, 10%, 20%, or 30%, relative to a baseline measurement (e.g., wherein the baseline measurement is determined prior to treatment). In some embodiments, a method treats the infection. In some embodiments, a method prevents the onset of an infection.

In some embodiments, a pathogen infection is an infection of the gastrointestinal tract, lungs, bloodstream, central nervous system, lymphatic system, and/or soft tissues of the subject.

In some embodiments, the oligosaccharide composition is administered in an amount sufficient, to reduce or prevent dysbiosis in the gut (e.g., small intestine, large intestine and/or colon) of the human subject.

In some embodiments, the oligosaccharide composition reduces the risk of an adverse effect of the pathogen on the human subject.

In some embodiments, the oligosaccharide composition is administered in an amount effective to: (a) reduce pathogen biomass (e.g., the number of pathogens and/or the number of drug- or antibiotic-resistance gene or MDR element carriers); (b) modulate (e.g., increase) the level of anti-microbial compounds produced by the subject (e.g., by the resident gut microbiota and/or the host (e.g., human cells)); (c) modulate the environment of the GI tract (e.g., small intestine, large intestine or colon), e.g. reducing the pH (e.g., by increasing production or levels of lactic acid, e.g. produced by the resident gut microbiota); (d) modulate (e.g., reduce) a conjugation property of a donor microbe of a drug- or antibiotic-resistance gene or MDR element or modulate (e.g., reduce) the ability of a donor microbe to share a drug- or antibiotic-resistance gene or MDR element with a recipient; (e) reduce the number of drug- or antibiotic-resistance gene or MDR element recipients; (f) reduce the copy number of a drug- or antibiotic-resistance gene or MDR element (e.g. total copy number, e.g. in a donor microbe); and/or (g) increase the fitness cost of the maintenance of antibiotic resistance genes or elements, in the human subject.

In some embodiments, the oligosaccharide composition is administered in an amount effective to: (a) decrease the relative abundance or absolute abundance of pathogens and/or drug- or antibiotic-resistance gene or MDR element carriers; and/or (b) increase the relative abundance or absolute abundance of commensal or beneficial bacteria.

In some embodiments, the pathogen is a bacterial microorganism or a fungal microorganism. In some embodiments, the pathogen is a drug or antibiotic resistant pathogen, optionally a multi-drug resistant (MDR) pathogen. In some embodiments, the pathogen is vancomycin resistant Enterococcus (VRE) or carbapenem resistant Enterobacteriaceae (CRE).

In some embodiments, the pathogen is VRE Enterococcus faecium. In some embodiments, the pathogen is CRE Escherichia coli or CRE Klebsiella pneumoniae. In some embodiments, the pathogen is Candida albicans, Candida glabrata, Candida krusei, Candida tropicalis, or Candida lusitaniae. In some embodiments, the pathogen is Clostridium difficile. In some embodiments, the pathogen is gram-positive bacteria or gram-negative bacteria. In some embodiments, the pathogen is a fungus. In some embodiments, the pathogen is Candida.

In some embodiments, a human subject has received cancer treatment. In some embodiments, a human subject is a transplant recipient. In some embodiments, a human subject has received immunosuppression. In some embodiments, a human subject has an auto-immune disease (e.g., systemic lupus erythematosus, rheumatoid arthritis, Sjögren's syndrome, or Crohn's disease). In some embodiments, a human subject has a hematological malignancy. In some embodiments, a human subject has cirrhosis. In some embodiments, a human subject has or is at risk of having end-stage liver disease (ESLD). In some embodiments, a human subject is preparing for or recovering from a gastrointestinal surgery. In some embodiments, a human subject is a patient in an intensive care unit (ICU). In some embodiments, a human subject has had multiple courses of antibiotics, and/or chronic use of antibiotics. In some embodiments, a human subject has a positive stool culture for Carbapenem-resistant Enterobacteriaciae (CRE), extended spectrum beta lactamase (ESBL) producing Enterobacteriaciae (ESBLE), and Vancomycin-resistant Enterococcus (VRE). In some embodiments, a human subject has a positive stool culture for Carbapenem-resistant Enterobacteriaciae (CRE). In some embodiments, a human subject has a positive stool culture for extended spectrum beta lactamase (ESBL) producing Enterobacteriaciae (ESBLE). In some embodiments, a human subject has a positive stool culture for Vancomycin-resistant Enterococcus (VRE). In some embodiments, a human subject has low diversity of bacterial communities in the gastrointestinal tract. In some embodiments, a human subject is a hematopoietic stem cell transplant (HSCT) recipient. In some embodiments, a human subject is a solid organ transplant recipient. In some embodiments, a human subject has recently had a central line-associated bloodstream infection (CLABSI). In some embodiments, a human subject has recently had a catheter-associated urinary tract infection (CAUTI). In some embodiments, a human subject has recently had a C. difficile infections).

In some aspects, the disclosure provides a method comprising

(a) identifying a human subject who (i) has received cancer treatment; (ii) is a transplant recipient; (iii) has received immunosuppression; (iv) has an auto-immune disease (e.g., systemic lupus erythematosus, rheumatoid arthritis, Sjögren's syndrome, or Crohn's disease); (v) has a hematological malignancy; (vi) has cirrhosis (e.g., including end-stage liver disease (ESLD)); (vii) is preparing for or recovering from a gastrointestinal surgery; (viii) is a patient in an intensive care unit (ICU); (ix) has had multiple courses of antibiotics, and/or chronic use of antibiotics; (x) has a positive stool culture for Carbapenem-resistant Enterobacteriaciae (CRE), extended spectrum beta lactamase (ESBL) producing Enterobacteriaciae (ESBLE), and/or Vancomycin-resistant Enterococcus (VRE); (xi) has low diversity of bacterial communities in the gastrointestinal tract; (xii) has increased levels of a drug or antibiotic resistant pathogen (e.g., VRE, CRE, Candida, or Clostridium difficile), gram-positive bacteria or gram-negative bacteria; and/or (xiii) has recently had a central line-associated bloodstream infection (CLABSI), a catheter-associated urinary tract infection (CAUTI), or a C. difficile infections); and

(b) treating the subject with an effective amount of an oligosaccharide composition as described herein.

In some embodiments, a method further comprises administering to the human subject a population of commensal or probiotic bacteria.

In some embodiments, a human subject is a patient having a gut microbiome devoid of any detectable levels of commensal bacteria.

In some embodiments, a method further comprises administering to the human subject antibiotics (e.g., broad spectrum antibiotics) or other standard-of-care treatment concurrent with the oligosaccharide composition.

In some embodiments, the subject has been treated with antibiotics (e.g., broad spectrum antibiotics) or other standard-of-care treatment prior to administration with the oligosaccharide composition.

In some embodiments, the oligosaccharide composition is administered to the subject one to twenty-eight days before a cancer treatment, surgery (e.g., transplant, e.g., hematopoietic stem cell), or admission to an intensive care unit. In some embodiments, the oligosaccharide composition is administered to the subject one to twenty-eight days after a cancer treatment, surgery (e.g., transplant, e.g., hematopoietic stem cell), or admission to an intensive care unit.

In some embodiments, the oligosaccharide composition is administered to the subject at least one to twenty-eight days after onset of a pathogen infection.

In some embodiments, the oligosaccharide composition is administered to the intestines (e.g., the large intestine).

In some embodiments, the oligosaccharide composition is self-administered to the subject. In some embodiments, the oligosaccharide composition is formulated as a pharmaceutical composition for oral delivery. In some embodiments, the oligosaccharide composition is orally administered to the subject. In some embodiments, the oligosaccharide composition is formulated as a pharmaceutical composition for delivery by a feeding tube. In some embodiments, the oligosaccharide composition is administered to the subject by a feeding tube.

In some embodiments, the oligosaccharide composition is administered to the subject once per day or twice per day.

In some aspects, the disclosure provides a method of reducing the relative or absolute abundance of pathogens in the gastrointestinal tract of a human subject, the method comprising administering to the gastrointestinal tract of the subject an effective amount of an oligosaccharide composition, wherein the oligosaccharide composition comprises a plurality of oligosaccharides selected from Formula (I), Formula (II), and Formula (III):

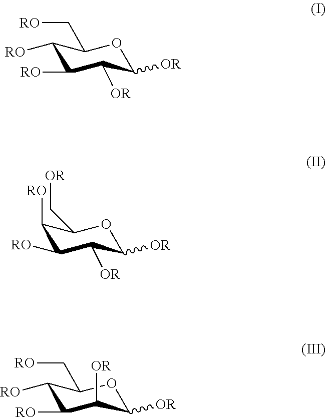

wherein each R independently is selected from hydrogen, and Formulae (Ia), (Ib), (Ic), (Id), (IIa), (IIb), (IIc), (IId), (IIIa), (IIIb), (IIIc), (IIId):

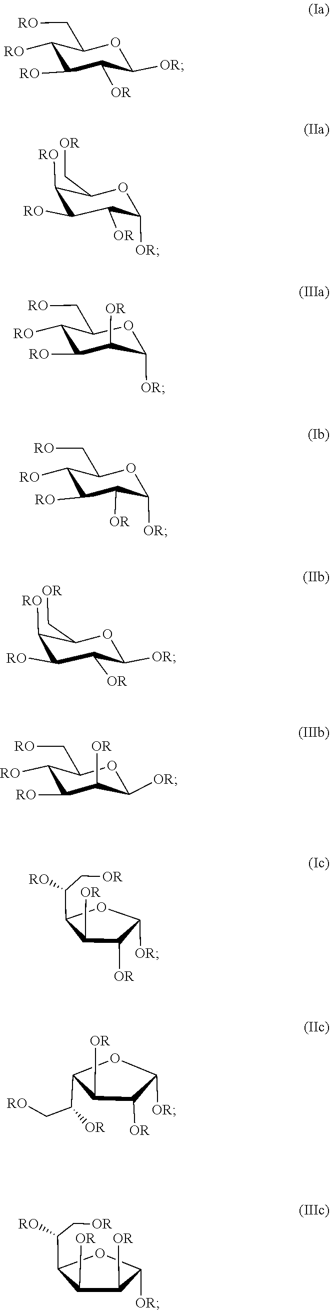

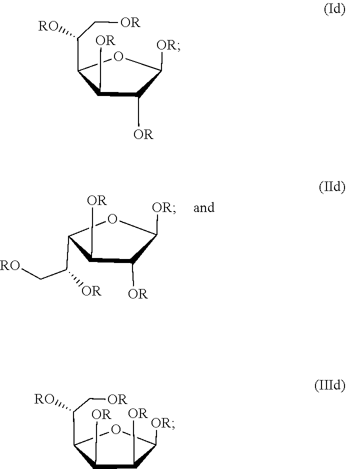

wherein each R independently is as defined above;

wherein the oligosaccharide composition is produced by a process comprising:

(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and

(b) promoting acid catalyzed oligosaccharide formation in the reaction mixture by transferring sufficient heat to the reaction mixture to maintain the reaction mixture at its boiling point.

In some embodiments, step (b) comprises promoting acid catalyzed oligosaccharide formation in the reaction mixture by transferring sufficient heat to the reaction mixture to maintain the reaction mixture at its boiling point until the weight percent of total monomer content in the oligosaccharide composition is in a range of 2% to 20%, wherein the total monomer content comprises dextrose monomer, galactose monomer, and/or mannose monomer.

In some aspects, the disclosure relates to a oligosaccharide composition (which may be useful as a microbiome metabolic therapy) that comprises a plurality of oligosaccharides selected from Formula (I) Formula (II) and Formula (III):

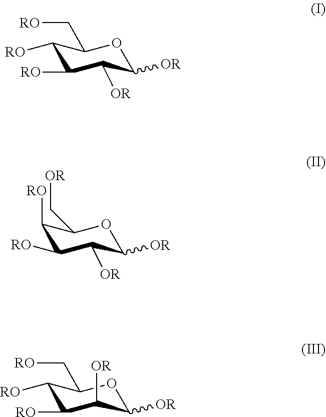

wherein each R independently is selected from hydrogen, and Formulae (Ia), (Ib), (Ic), (Id), (IIa), (IIb), (IIc), (IId), (IIIa), (IIIb), (IIIc), (IIId):

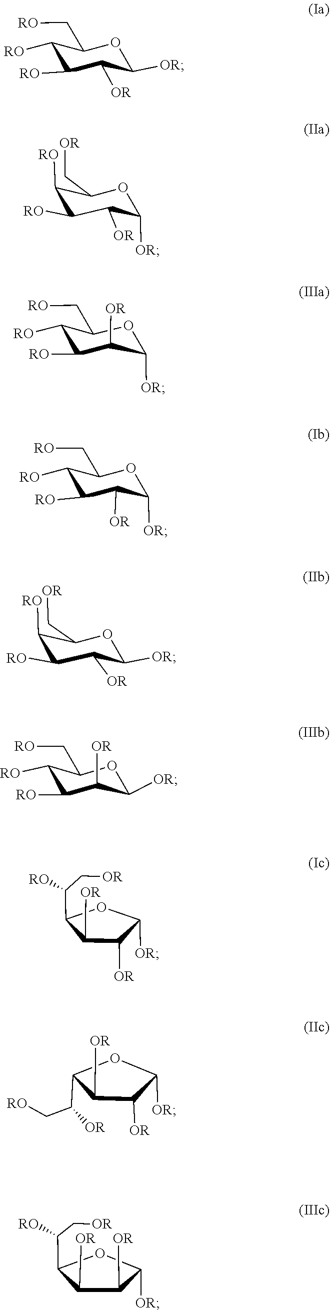

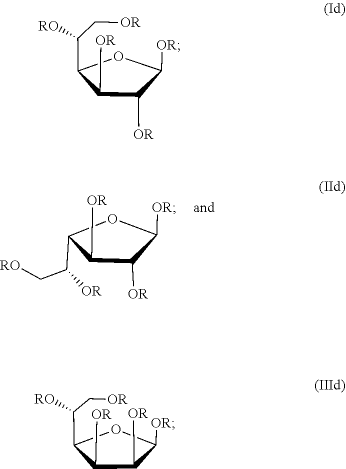

wherein each R independently is as defined above. In some embodiments, the oligosaccharide composition is produced by a process comprising:

(a) heating a preparation comprising dextrose monomer, galactose monomer, and mannose monomer wherein the molar ratio of dextrose to galactose is about 1:1 and the molar ratio of dextrose to mannose is about 4.5:1 under agitation conditions, to a temperature in a range of 100° C. to 160° C.;

(b) loading the preparation with an acid catalyst comprising positively charged hydrogen ions, thereby forming a reaction mixture; and

(c) maintaining the reaction mixture at atmospheric pressure, at a temperature in a range of 100° C. to 160° C., under conditions that promote acid catalyzed oligosaccharide composition formation, until the weight percent of total monomer content in the oligosaccharide composition is in a range of 2% to 14%, wherein the total monomer content comprises dextrose monomer, galactose monomer, and/or mannose monomer;

(d) quenching the reaction mixture, for example, using water, while bringing the temperature of the reaction mixture to a temperature in the range of 55° C. to 95° C. (e.g., 85° C., 90° C.); and optionally,

(e) separating oligosaccharide compositions from the acid catalyst;

thereby obtaining the oligosaccharide composition.

In another aspect the disclosure relates to a oligosaccharide composition (which may be useful as a microbiome metabolic therapy) that comprises a plurality of oligosaccharides selected from Formula (I), Formula (II), and Formula (III):

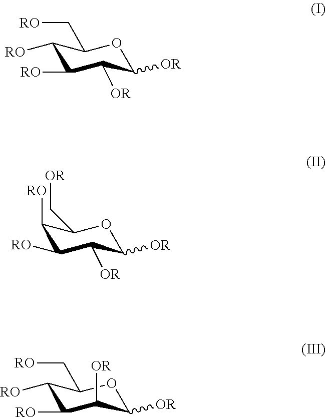

wherein each R independently is selected from hydrogen, and Formulae (IIa), (IIb), (IIc), (IId), (IIIa), (IIIb), (IIIc), (IIId):

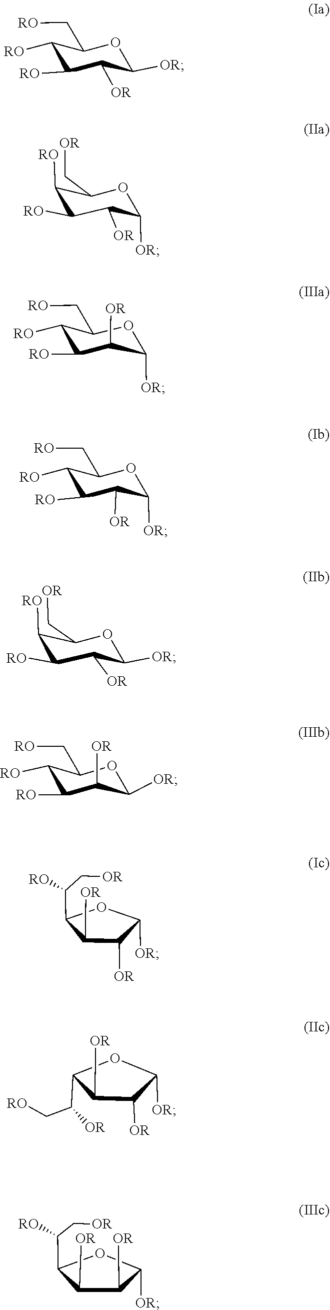

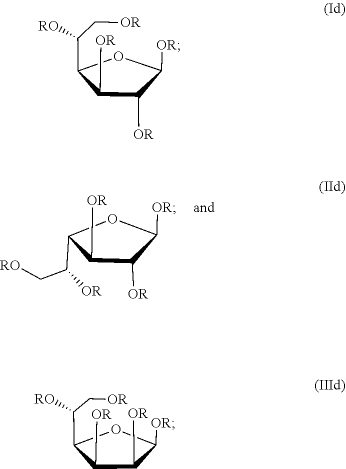

wherein each R independently is as defined above;

wherein the oligosaccharide composition is produced by a process comprising:

(a) heating a preparation comprising dextrose monomer, galactose monomer, and mannose monomer wherein the molar ratio of dextrose to galactose is about 1:1 and the molar ratio of dextrose to mannose is about 4.5:1 under agitation conditions, to a temperature in a range of 100° C. to 160° C.;

(b) loading the preparation with an acid catalyst comprising positively charged hydrogen ions, thereby forming a reaction mixture;

(c) maintaining the reaction mixture at its boiling point, at a pressure in the range of 0.5-1.5 atm, under conditions that promote acid catalyzed oligosaccharide formation, until the weight percent of total monomer content in the oligosaccharide composition is in a range of 2% to 14%, wherein the total monomer content comprises dextrose monomer, galactose monomer, and/or mannose monomer;

(d) quenching the reaction mixture, for example, using water, while bringing the temperature of the reaction mixture to a temperature in the range of 55° C. to 95° C. (e.g., 85° C., 90° C.); and optionally,

(e) separating oligosaccharides from the acid catalyst;

thereby obtaining the oligosaccharide composition.

### BRIEF DESCRIPTION OF THE DRAWINGS

FIG. 1 depicts exemplary uses of oligosaccharide compositions to reduce the colonization of pathogens in an ex vivo assay (closed system) and an in vivo clinical setting (open system). Bacteria depictions in dark color (red) symbolize pathogens or pathobionts. Bacteria depictions in light color (blue) symbolize commensal bacteria.

FIG. 2 provides graphs showing reduction in pathogen growth in cultures of single pathogen strains (Clostridium difficile strains) incubated in the presence of samples of a selected oligosaccharide composition.

FIG. 3 provides graphs showing reduction in pathogen growth in cultures of single pathogen strains (VRE Enterococcus faecium) incubated in the presence of samples of a selected oligosaccharide composition.

FIG. 4 provides graphs showing reduction in pathogen growth in cultures of single pathogen strains (CRE Escherichia coli, CRE Klebsiella pneumoniae) incubated in the presence of samples of a selected oligosaccharide composition.

FIG. 5 provides graphs showing reduction in pathogen growth in cultures of single pathogen strains (Candida albicans, Candida glabrata, Candida krusei, Candida tropicalis) incubated in the presence of samples of a selected oligosaccharide composition.

FIGS. 6A-6B provide graphs showing reduction in pathogen growth in cultures of single pathogen strains (Candida lusitaniae) incubated in the presence of samples of a selected oligosaccharide composition. FIG. 6A provides graphs specific for ATCC 66035 strain. FIG. 6B provides graphs specific for ATCC 42720 strain.

FIGS. 7A-7B provide graphs showing reduction in pathogen growth (normalized to water controls) in an ex vivo pathogen reduction assay where fecal samples from 11 ICU patients were incubated with the selected oligosaccharide composition. FIG. 7A is a graph showing reduction of pathogens in fecal samples spiked with carbapenem-resistant Enterobacteriaceae. FIG. 7B is a graph showing reduction of pathogens in fecal samples spiked with vancomycin-resistant Enterococcaceae.

FIGS. 8A-8B provide graphs showing reduction in pathogen growth (normalized to water controls) in an ex vivo pathogen reduction assay where fecal samples from hepatic encephalopathy (HE) patients were incubated with the selected oligosaccharide composition. FIG. 8A is a graph showing reduction of pathogens in fecal samples spiked with carbapenem-resistant Enterobacteriaceae. FIG. 8B is a graph showing reduction of pathogens in fecal samples spiked with vancomycin-resistant Enterococcaceae.

FIG. 9 depicts a SEC-HPLC chromatogram of a selected oligosaccharide composition using the method provided in Example 15.

FIG. 10 depicts an overlay of SEC-HPLC chromatograms of standard saccharides for use in Example 17.

FIG. 11 is a HSQC NMR spectra of the selected oligosaccharide composition.

FIG. 12 provides a graph showing the microbial compositions of fecal samples collected from ICU patients and fecal samples collected from healthy subjects. Presented are relative proportions of discrete bacterial taxa (genus-level) in each fecal sample.

FIGS. 13A-13B provide graphs showing reduction in relative proportions of pathogenic microbes (e.g., VRE E. faecium, Enterobacteriales) and increase in relative proportions of commensal microbes in an ex vivo pathogen reduction assay. Fecal samples that had been spiked with vancomycin-resistant Enterococcaceae or carbapenem-resistant Enterobacteriaceae were incubated with the selected oligosaccharide composition, FOS, or water.

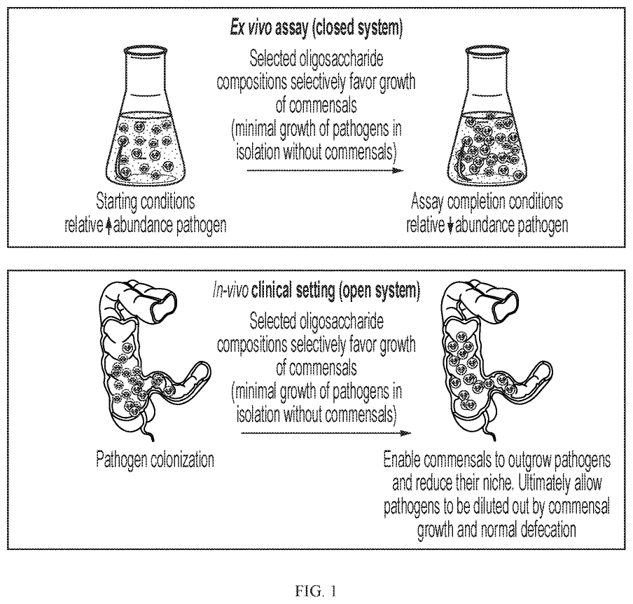

### DETAILED DESCRIPTION OF INVENTION

Aspects of the disclosure relate to oligosaccharide compositions that are effective for reducing pathogen levels, abundance and/or colonization and colonization in a subject.

Some aspects of the disclosure are based on the results of an extensive screening effort that was performed to identify oligosaccharide compositions that are capable of modulating, e.g., reducing, levels of pathogens in a subject. Hundreds of unique oligosaccharide compositions were assayed for their effect on pathogen levels. The oligosaccharide compositions examined in the screen were produced using different saccharide monomers, e.g., dextrose monomers, xylose monomers, etc., and under conditions involving differing reaction temperatures, for varying periods of time, and/or in the presence of different catalyst conditions.

From this screening effort, a selected oligosaccharide composition was identified as a highly effective modulator of pathogen levels and colonization. Accordingly, in some embodiments, this oligosaccharide composition is particularly useful for treating subjects having high levels of pathogen colonization in their GI tract (e.g., subjects colonized with pathogens in their intestines) and/or receiving broad spectrum antibiotics. Further aspects of the disclosure, including a description of defined terms, are provided below.

### I. Definitions

Agitation conditions: As used herein, the term “agitation conditions” refers to conditions that promote or maintain a substantially uniform or homogeneous state of a mixture (e.g., a reaction mixture comprising dextrose monomer, galactose monomer, and mannose monomer) with respect to dispersal of solids (e.g., solid catalysts), uniformity of heat transfer, or other similar parameters. Agitation conditions generally include stirring, shaking, and/or mixing of a reaction mixture. In some embodiments, agitation conditions may include the addition of gases or other liquids into a solution. In some embodiments, agitation conditions are used to maintain substantially uniform or homogenous distribution of a catalyst, e.g., an acid catalyst. In some embodiments, a monosaccharide preparation is heated in the presence of an acid catalyst under suitable conditions to achieve homogeneity and uniform heat transfer in order to synthesize an oligosaccharide composition.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Dextrose monomer: As used herein, the term “dextrose monomer” refers to a D-isomer of a glucose monomer, known as D-glucose. In some embodiments, a dextrose monomer is dextrose monohydrate or 70DS corn syrup.

Effective amount: As used herein, the term “effective amount” refers to an administered amount or concentration of an oligosaccharide composition that is necessary and sufficient to elicit a biological response, e.g., in a subject or patient. In some embodiments, an effective amount of an oligosaccharide composition is capable of modulating, e.g., increasing or decreasing, the activity or levels of an enzyme in a subject. In some embodiments, an effective amount of an oligosaccharide composition is capable of modulating, e.g., increasing or decreasing, the processing of a metabolite. In some embodiments, an effective amount of an oligosaccharide composition is capable of modulating, e.g., increasing or decreasing, the concentration or number of at least one microbial species. In some embodiments, an effective amount of an oligosaccharide composition is capable of modulating, e.g., decreasing, the symptoms of a disease associated with elevated pathogen colonization in a subject (e.g., the severity or number of symptoms). In some embodiments, an effective amount of an oligosaccharide composition is capable of reducing the acquisition of, colonization of, or reducing the reservoir of a pathogen (e.g., a drug or antibiotic resistant pathogen, or an MDR pathogen) in a subject. In some embodiments, an effective amount of an oligosaccharide composition is capable of treating a subject having intestinal colonization with a pathogen, e.g., CRE or VRE.

Galactose monomer: As used herein, the term “galactose monomer” generally refers to a D-isomer of a galactose monomer, known as D-galactose.

Mannose monomer: As used herein, the term “mannose monomer” generally refers to a D-isomer of a mannose monomer, known as D-mannose.

Monosaccharide Preparation: As used herein, the term “monosaccharide preparation” refers to a preparation that comprises two or more monosaccharides (e.g., dextrose monomer, galactose monomer, and mannose monomer). In some embodiments, a monosaccharide preparation comprises dextrose monomers, galactose monomers, and mannose monomers.

Oligosaccharide: As used herein, the term “oligosaccharide” (which may be used interchangeably with the term “glycan” in some contexts) refers to a saccharide molecule comprising at least two monosaccharides (e.g., dextrose monomers, galactose monomers, mannose monomers) linked together via a glycosidic bond (having a degree of polymerization (DP) of at least 2 (e.g., DP2+)). In some embodiments, an oligosaccharide comprises at least two, at least three, at least four, at least five, at least six, at least seven, at least eight, at least nine, or at least ten monosaccharides subunits linked by glycosidic bonds. In some embodiments, an oligosaccharide is in the range of 3-20, 4-16, 5-15, 8-12, 5-25, 10-25, 20-50, 40-80, or 75-100 monosaccharides linked by glycosidic bonds. In some embodiments, an oligosaccharide comprises at least one 1,2; 1,3; 1,4; and/or 1,6 glycosidic bond. Oligosaccharides may be linear or branched. Oligosaccharides may have one or more glycosidic bonds that are in alpha-configurations and/or one or more glycosidic bonds that are in beta-configurations.

Pharmaceutical Composition: As used herein, a “pharmaceutical composition” refers to a composition having pharmacological activity or other direct effect in the mitigation, treatment, or prevention of disease, and/or a finished dosage form or formulation thereof and is for human use. A pharmaceutical composition or pharmaceutical preparation is typically produced under good manufacturing practices (GMP) conditions. Pharmaceutical compositions or preparations may be sterile or non-sterile. If non-sterile, such pharmaceutical compositions or preparations typically meet the microbiological specifications and criteria for non-sterile pharmaceutical products as described in the U.S. Pharmacopeia (USP) or European Pharmacopoeia (EP). Any oligosaccharide composition described herein may be formulated as a pharmaceutical composition.

Subject: As used herein, the term “subject” refers to a human subject or patient. Subjects may include a newborn (a preterm newborn, a full-term newborn), an infant up to one year of age, young children (e.g., 1 yr to 12 yrs), teenagers, (e.g., 13-19 yrs), adults (e.g., 20-64 yrs), and elderly adults (65 yrs and older). In some embodiments, a subject is of a pediatric population, or a subpopulation thereof, including neonates (birth to 1 month), infants (1 month to 2 years), developing children (2-12 years), and adolescents (12-16 years). In some embodiments, a subject is a healthy subject. In some embodiments, a subject is a patient having higher abundance of pathogen relative to a healthy subject, e.g., a subject colonized with a pathogen (e.g., CRE and/or VRE pathogens) in their gastrointestinal tract (e.g., their colon or intestines.). In some embodiments, a subject is a patient receiving broad spectrum antibiotics. In some embodiments, the subject is particularly susceptible to pathogen infection, e.g., the subject is critically-ill and/or immunocompromised. In some embodiments, the subject is a patient having a lower abundance of commensal bacteria relative to a healthy subject in their gastrointestinal tract (e.g., their colon or intestines).

Treatment and Treating: As used herein, the terms “treating” and “treatment” refer to the administration of a composition to a subject (e.g., a symptomatic subject afflicted with an adverse condition, disorder, or disease) so as to affect a reduction in severity and/or frequency of a symptom, eliminate a symptom and/or its underlying cause, and/or facilitate improvement or remediation of damage, and/or preventing an adverse condition, disorder, or disease in an asymptomatic subject who is susceptible to a particular adverse condition, disorder, or disease, or who is suspected of developing or at risk of developing the condition, disorder, or disease. In some embodiments, treating a subject with an oligosaccharide composition reduces the relative or absolute abundance of pathogens in the gastrointestinal tract of the subject. In some embodiments, treating a subject with an oligosaccharide composition slows or reduces the rate of a pathogen infection (e.g., in the gastrointestinal tract). In some embodiments, treating a subject with an oligosaccharide composition prevents the onset of a pathogen infection (e.g., in the gastrointestinal tract). In some embodiments, the oligosaccharide composition treats an infection (e.g., bacterial infection). In some embodiments, the oligosaccharide composition treats a localized infection. In some embodiments, the oligosaccharide composition treats a nascent infection. In some embodiments, the oligosaccharide composition treats a systemic infection, e.g., a systemic C. diff infection. However, in some embodiments, the selected oligosaccharide composition prevents an infection, e.g., a localized, nascent, or systemic infection. In some embodiments, treating a subject with an oligosaccharide composition eliminates a pathogen infection and/or reduces pathogenic load (e.g., in the gastrointestinal tract). In some embodiments, a subject has or is at risk of a pathogenic (e.g., bacterial or fungal) infection (e.g., an infection of the gastrointestinal tract). In some embodiments, a subject has recently had and/or recovered pathogenic (e.g., bacterial or fungal) infection (e.g., an infection of the gastrointestinal tract). In some embodiments, a subject is an immunocompromised subject (e.g., a hematopoietic stem cell transplantation (HSCT) patient). In some embodiments, a subject is a healthy subject.

### II. Oligosaccharide Compositions

Provided herein are oligosaccharide compositions, and their methods of use for modulating levels of pathogens in a human subject.

In one aspect, oligosaccharide compositions are provided herein that comprise a plurality of oligosaccharides selected from Formula (I), Formula (II), and Formula (III):

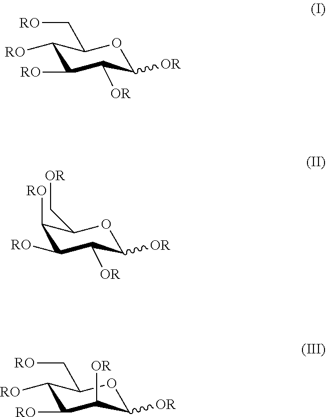

wherein each R independently is selected from hydrogen, and Formulae (Ia), (Ib), (Ic), (Id), (IIa), (IIb), (IIc), (IId), (IIIa), (IIIb), (IIIc), (IIId):

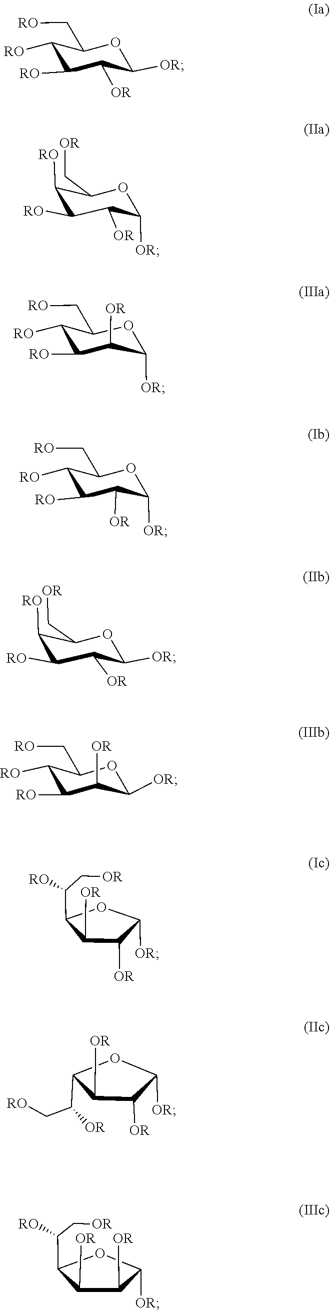

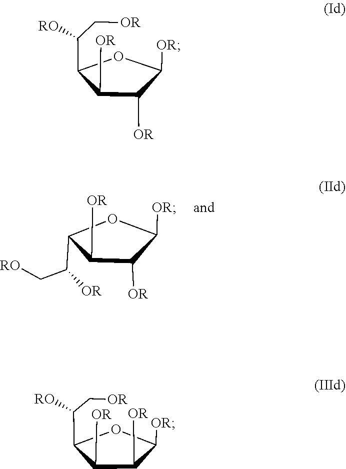

wherein each R independently is as defined above.

In some embodiments, oligosaccharide compositions are produced by a process that initially involves heating a preparation comprising dextrose monomers, galactose monomers, and mannose monomers to a temperature in a range of 100° C. to 160° C., 100° C. to 120° C., 110° C. to 130° C., 120° C. to 140° C., 130° C. to 150° C., or about 140° C. The ratio of dextrose monomers to galactose monomers may be 1:1. The ratio of dextrose monomers to mannose monomers may be 4.5:1. The ratio of galactose monomers to mannose monomers may be 4.5:1. Heating may be performed under agitation conditions. Heating may comprises gradually increasing the temperature (e.g., from room temperature) to about 130° C., about 135° C. about 140° C. about 145° C., or about 150° C. under suitable conditions to achieve homogeneity and uniform heat transfer. An acid catalyst comprising positively charged hydrogen ions is added to the preparation (e.g., following heating). In some embodiments, the acid catalyst is a solid catalyst. In some embodiments, the catalyst is a strong acid cation exchange resin having one or more physical and chemical properties according to Table 1. In some embodiments, the catalyst comprises >3.0 mmol/g sulfonic acid moieties and <1.0 mmol/gram cationic moieties. In certain embodiments, the catalyst has a nominal moisture content of 45-50 weight percent. In certain embodiments, the catalyst is added at the same time as the dextrose monomers, galactose monomers, and mannose monomers. In some embodiments, after loading of the catalyst with the preparation, the resultant reaction mixture is held at atmospheric pressure and at a temperature in a range of 100° C. to 160° C., 100° C. to 120° C., 110° C. to 130° C., 120° C. to 140° C., 130° C. to 150° C., or about 140° C. under conditions that promote acid catalyzed oligosaccharide formation. In some embodiments, once the weight percent of total monomer content in the oligosaccharide composition (total monomer content comprises the amount of dextrose monomer, galactose monomer, and/or mannose monomer) is in a range of 2-14% (optionally 2-5%, 4-8%, 7-10%, 9-14%, or 12-14%), the reaction mixture is quenched. Quenching typically involves using water (e.g., deionized water) to dilute the reaction mixture, and gradually decrease the temperature of the reaction mixture to 55° C. to 95° C. In some embodiments, the water used for quenching is about 95° C. The water may be added to the reaction mixture under conditions sufficient to avoid solidifying the mixture. In certain embodiments, water may be removed from the reaction mixture by evaporation. In some embodiments, the reaction mixture may contain 93-94 weight percent dissolved solids. Finally, to obtain a purified oligosaccharide composition, the composition is generally separated from the acid catalyst, typically by diluting the quenched reaction mixture with water to a concentration of about 45-55 weight percent and a temperature of below about 85° C. and then passing the mixture through a filter or a series of chromatographic resins. In certain embodiments, the filter used is a 0.45 μm filter. Alternatively, a series of chromatographic resins may be used and generally involves a cationic exchange resin, an anionic exchange resin, and/or a decolorizing polymer resin. In some embodiments, any or all of the types of resins may be used one or more times in any order. In some embodiments, the oligosaccharide composition comprises water at a level below that which is necessary for microbial growth upon storage at room temperature. In certain embodiments, the mean degree of polymerization of all oligosaccharides is in a range of 7-15.5, optionally 11-15. In some embodiments, the oligosaccharide composition comprises water in a range of 45-55 weight percent. In some embodiments, the oligosaccharide composition comprises oligosaccharides that have a MWw (weight-average molecular weight) (g/mol) in a range of 1905-2290. In some embodiments, the oligosaccharide composition comprises oligosaccharides that have a MWn (number-average molecular weight) (g/mol) in a range of 1030-1095. In some embodiments, a solution comprising the oligosaccharide composition has a pH in a range of 2.50-3.50. In some embodiments, the oligosaccharide composition comprises oligomers having two or more repeat units (DP2+) in a range of 86-96 weight percent.

Further, in some embodiments, oligosaccharide compositions may be de-monomerized. In some embodiments, de-monomerization involves the removal of residual saccharide monomers. In some embodiments, de-monomerization is performed using chromatographic resin. Accordingly, in some embodiments, different compositions can be prepared depending upon the percent of monomer present. In some embodiments, oligosaccharide compositions are de-monomerized to a monomer content of about 1%, about 3%, about 5%, about 10%, or about 15%. In some embodiments, oligosaccharide compositions are de-monomerized to a monomer content of about 1-3%, about 3-6%, about 5-8%, about 7-10%, or about 10-15%. In one embodiment, the oligosaccharide compositions is de-monomerized to a monomer content of less than 1%. In one embodiment, the oligosaccharide composition is de-monomerized to a monomer content between about 7% and 10%. In one embodiment, the oligosaccharide compositions is de-monomerized to a monomer content between about 1% and 3%. In one embodiment, de-monomerization is achieved by osmotic separation. In a second embodiment de-monomerization is achieved by tangential flow filtration (TFF). In a third embodiment de-monomerization is achieved by ethanol precipitation.

In some embodiments, oligosaccharide compositions with different monomer contents may also have different measurements for total dietary fiber, moisture, total dietary fiber (dry basis), or percent Dextrose Equivalent (DE). In some embodiments, total dietary fiber is measured according to the methods of AOAC 2011.25. In some embodiments, moisture is measured by using a vacuum oven at 60° C. In some embodiments, total dietary fiber is (dry basis) is calculated. In some embodiments, the percent DE is measured according to the Food Chemicals Codex (FCC).

In some embodiments, the oligosaccharide compositions have a total dietary fiber content of 87.4 percent (on dry basis). In some embodiments, the oligosaccharide compositions have a total dietary fiber content of 81.9-93.0, 82-85, 85-88, 88-90, or 90-93 percent (on dry basis). In some embodiments, the oligosaccharide compositions have a total dietary fiber content of about 82, about 85, about 87, about 90, or about 93 percent (on dry basis). In some embodiments, the oligosaccharide compositions have a total dietary fiber content of 78-97 percent (on dry basis). In some embodiments, the oligosaccharide compositions have a total dietary fiber content of 82-93 percent (on dry basis). In some embodiments, the oligosaccharide compositions have a total dietary fiber content of 14.5-100 percent (on dry basis). In some embodiments, the oligosaccharide compositions have a total dietary fiber content of 34-94 percent (on dry basis).

In some embodiments, the oligosaccharide compositions have a total reducing sugar content (Dextrose Equivalence (DE) (dry solids)) of 6.5-35 percent. In some embodiments, the oligosaccharide compositions have a total reducing sugar content (Dextrose Equivalence (DE) (dry solids)) of 12-29 percent. In some embodiments, the oligosaccharide compositions have a total reducing sugar content (Dextrose Equivalence (DE) (dry solids)) of 5-40, 5-30, 5-25, 10-30, 10-25, 10-20, 15-30, 15-25, or 15-20 percent.

In some embodiments, production of oligosaccharides compositions according to methods provided herein can be performed in a batch process or a continuous process. For example, in one embodiment, oligosaccharide compositions are produced in a batch process, where the contents of the reactor are subjected to agitation conditions (e.g., continuously mixed or blended), and all or a substantial amount of the products of the reaction are removed (e.g., isolated and/or recovered).

In certain embodiments, the methods of using the catalyst are carried out in an aqueous environment. One suitable aqueous solvent is water, which may be obtained from various sources. Generally, water sources with lower concentrations of ionic species (e.g., salts of sodium, phosphorous, ammonium, or magnesium) may be used, in some embodiments, as such ionic species may reduce effectiveness of the catalyst. In some embodiments where the aqueous solvent is water, the water has less than 10% of ionic species (e.g., salts of sodium, phosphorous, ammonium, magnesium). In some embodiments where the aqueous solvent is water, the water has a resistivity of at least 0.1 megaohm-centimeters, of at least 1 megaohm-centimeters, of at least 2 megaohm-centimeters, of at least 5 megaohm-centimeters, or of at least 10 megaohm-centimeters.

In some embodiments, as reactions of methods provided herein progress, water (such as evolved water) is produced with each glycosidic coupling of the one or more saccharide monomer. In certain embodiments, the methods described herein may further include monitoring the amount of water present in the reaction mixture and/or the ratio of water to monomer or catalyst over a period of time. Thus, in some embodiments, the water content of the reaction mixture may be altered over the course of the reaction, for example, removing evolved water produced. Appropriate methods may be used to remove water (e.g., evolved water) in the reaction mixture, including, for example, by evaporation, such as via distillation. In some embodiments, the method comprises including water in the reaction mixture. In certain embodiments, the method comprises removing water from the reaction mixture through evaporation.

In some embodiments, the ratio of dextrose monomer to galactose monomer is about 1:2, 1:1.5, 1:1.4, 1:1.3, 1:1.2, 1:1.1, 1:1, 1.1:1, 1.2:1, 1.3:1, 1.4:1, 1.5:1, or 2:1. In some embodiments, the ratio of dextrose monomer to galactose monomer is about 1:1.

In some embodiments, the ratio of dextrose monomer to mannose monomer is about 1:1, 1.5:1, 2:1, 2.5:1, 3:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, or 5.5:1. In some embodiments, the ratio of dextrose monomer to mannose monomer is about 4.5:1.

In some embodiments, the ratio of galactose monomer to mannose monomer is about 1:1, 1.5:1, 2:1, 2.5:1, 3:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, or 5.5:1. In some embodiments, the ratio of galactose monomer to mannose monomer is about 4.5:1.

In some embodiments, the monosaccharide preparation comprises about 30-60% dextrose monomer, about 30-60% galactose monomer, and 1-25% mannose monomer. In some embodiments, the monosaccharide preparation comprises about 30-60% dextrose monomer, about 30-60% galactose monomer, and about 5-15% mannose monomer. In some embodiments, the monosaccharide preparation comprises about 40-50% dextrose monomer, about 40-50% galactose monomer, and about 5-15% mannose monomer. In some embodiments, the monosaccharide preparation comprises about 45% dextrose monomer, about 45% galactose monomer, and about 10% mannose monomer.

In certain embodiments, the preparation is loaded with an acid catalyst comprising positively charged hydrogen ions. In some embodiments, an acid catalyst is a solid catalyst (e.g., Dowex Marathon C). In some embodiments, an acid catalyst is a soluble catalyst (e.g., citric acid).

In some embodiments, the molar ratio of positively charged hydrogen ions to total dextrose monomer, galactose monomer, and mannose monomer content is in an appropriate range. In some embodiments, the molar ratio of positively charged hydrogen ions to total dextrose monomer, galactose monomer, and mannose monomer content is in a range of 0.01 to 0.1, 0.02 to 0.08, 0.03 to 0.06, or 0.05 to 0.06. In some embodiments, the molar ratio of positively charged hydrogen ions to total dextrose monomer, galactose monomer, and mannose monomer content is in a range of 0.003 to 0.01, 0.005 to 0.02, 0.01 to 0.02, 0.01 to 0.03, 0.02 to 0.03, 0.02 to 0.04, 0.03 to 0.05, 0.03 to 0.08, 0.04 to 0.07, 0.05 to 0.1, 0.05 to 0.2, 0.1 to 0.2, 0.1 to 0.3, or 0.2 to 0.3. In some embodiments, the molar ratio of positively charged hydrogen ions to total dextrose monomer, galactose monomer, and mannose monomer content is in a range of 0.050 to 0.052. In some embodiments, the molar ratio of positively charged hydrogen ions to total dextrose monomer, galactose monomer, and mannose monomer content is in a range of 0.020 to 0.035. In some embodiments, the molar ratio of positively charged hydrogen ions to total dextrose monomer, galactose monomer, and mannose monomer content is 0.028.

In some embodiments, the molar ratio of soluble acid catalyst (e.g., citric acid catalyst) to total dextrose monomer, galactose monomer, and mannose monomer content is in an appropriate range. In some embodiments, the molar ratio of soluble acid catalyst (e.g., citric acid catalyst) to total dextrose monomer, galactose monomer, and mannose monomer content is in a range of 0.01 to 0.1, 0.02 to 0.08, 0.03 to 0.06, or 0.05 to 0.06. In some embodiments, the molar ratio of soluble acid catalyst (e.g., citric acid catalyst) to total dextrose monomer, galactose monomer, and mannose monomer content is in a range of 0.003 to 0.01, 0.005 to 0.02, 0.01 to 0.02, 0.01 to 0.03, 0.02 to 0.03, 0.02 to 0.04, 0.03 to 0.05, 0.03 to 0.08, 0.04 to 0.07, 0.05 to 0.1, 0.05 to 0.2, 0.1 to 0.2, 0.1 to 0.3, or 0.2 to 0.3. In some embodiments, the molar ratio of soluble acid catalyst (e.g., citric acid catalyst) to total dextrose monomer, galactose monomer, and mannose monomer content is in a range of 0.050 to 0.052. In some embodiments, the molar ratio of soluble acid catalyst (e.g., citric acid catalyst) to total dextrose monomer, galactose monomer, and mannose monomer content is in a range of 0.020 to 0.035. In some embodiments, the molar ratio of soluble acid catalyst (e.g., citric acid catalyst) to total dextrose monomer, galactose monomer, and mannose monomer content is 0.028.

In some embodiments, water is added to the reaction mixture to quench the reaction by bringing the temperature of the reaction mixture to 100° C. or below. In some embodiments, the water used for quenching is deionized water. In some embodiments, the water used for quenching is USP water. In some embodiments, the water has a temperature of about 60° C. to about 100° C. In certain embodiments, the water used for quenching is about 95° C. In some embodiments, the water is added to the reaction mixture under conditions sufficient to avoid solidifying the mixture.

The viscosity of the reaction mixture may be measured and/or altered over the course of the reaction. In general, viscosity refers to a measurement of a fluid's internal resistance to flow (e.g., “thickness”) and is expressed in centipoise (cP) or pascal-seconds. In some embodiments, the viscosity of the reaction mixture is between about 100 cP and about 95,000 cP, about 5,000 cP and about 75,000 cP, about 5,000 and about 50,000 cP, or about 10,000 and about 50,000 cP. In certain embodiments, the viscosity of the reaction mixture is between about 50 cP and about 200 cP.

In some embodiments, oligosaccharide compositions provided herein may be subjected to one or more additional processing steps. Additional processing steps may include, for example, purification steps. Purification steps may include, for example, separation, demonomerization, dilution, concentration, filtration, desalting or ion-exchange, chromatographic separation, or decolorization, or any combination thereof.

In certain embodiments, the methods described herein further include a dilution step. In some embodiments, deionized water is used for dilution. In certain embodiments, USP water is used for dilution. In certain embodiments, after dilution, the oligosaccharide composition comprises water in a range of about 5-75, 25-65, 35-65, 45-55, or 47-53 weight percent. In certain embodiments, after dilution, the oligosaccharide composition comprises water in a range of about 45-55 weight percent.

In some embodiments, the methods described herein further include a decolorization step. The one or more oligosaccharide compositions produced may undergo a decolorization step using appropriate methods, including, for example, treatment with an absorbent, activated carbon, chromatography (e.g., using ion exchange resin), and/or filtration (e.g., microfiltration).

In some embodiments, the one or more oligosaccharide compositions produced are contacted with a material to remove salts, minerals, and/or other ionic species. For example, in certain embodiments, the one or more oligosaccharide compositions produced are flowed through an anionic exchange column. In other embodiments, oligosaccharide compositions produced are flowed through an anionic/cationic exchange column pair.

In some embodiments, the methods described herein may further include a concentration step. For example, in some embodiments, the oligosaccharide compositions may be subjected to evaporation (e.g., vacuum evaporation) to produce a concentrated oligosaccharide composition. In other embodiments, the oligosaccharide compositions may be subjected to a spray drying step to produce an oligosaccharide powder. In certain embodiments, the oligosaccharide compositions may be subjected to both an evaporation step and a spray drying step. In some embodiments, the oligosaccharide compositions be subjected to a lyophilization (e.g., freeze drying) step to remove water and produce powdered product.

In some embodiments, the methods described herein further include a fractionation step. Oligosaccharide compositions prepared and purified may be subsequently separated by molecular weight using any method known in the art, including, for example, high-performance liquid chromatography, adsorption/desorption (e.g. low-pressure activated carbon chromatography), or filtration (for example, ultrafiltration or diafiltration). In certain embodiments, oligosaccharide compositions are separated into pools representing 60%, 65%, 70%, 75%, 80%, 85%, 90%, 95%, 97%, 98%, or greater than 98% short (about DP1-2), medium (about DP3-10), long (about DP11-18), or very long (about DP>18) species.

In certain embodiments, prepared oligosaccharide compositions are fractionated by adsorption onto a carbonaceous material and subsequent desorption of fractions by washing the material with mixtures of an organic solvent in water at a concentration of 1%, 5%, 10%, 20%, 50%, or 100%. In one embodiment, the adsorption material is activated charcoal. In another embodiment, the adsorption material is a mixture of activated charcoal and a bulking agent such as diatomaceous earth or Celite 545 in 5%, 10%, 20%, 30%, 40%, or 50% portion by volume or weight.

In further embodiments, prepared oligosaccharide compositions are separated by passage through a high-performance liquid chromatography system. In certain variations, prepared oligosaccharide compositions are separated by ion-affinity chromatography, hydrophilic interaction chromatography, or size-exclusion chromatography including gel-permeation and gel-filtration.

In some embodiments, catalyst is removed by filtration. In certain embodiments, a 0.45 μm filter is used to remove catalyst during filtration. In other embodiments, low molecular weight materials are removed by filtration methods. In certain variations, low molecular weight materials may be removed by dialysis, ultrafiltration, diafiltration, or tangential flow filtration. In certain embodiments, the filtration is performed in static dialysis tube apparatus. In other embodiments, the filtration is performed in a dynamic flow filtration system. In other embodiments, the filtration is performed in centrifugal force-driven filtration cartridges. In certain embodiments, the reaction mixture is cooled to below about 85° C. before filtration.

In certain embodiments, the mean degree of polymerization of all oligosaccharides is in a range of 6-16. In certain embodiments, the mean degree of polymerization of all oligosaccharides is in a range of 10-15. In certain embodiments, the mean degree of polymerization of all oligosaccharides is in a range of 9-16. In certain embodiments, the mean degree of polymerization of all oligosaccharides is in a range of 10.5-15. In certain embodiments, the mean degree of polymerization of all oligosaccharides is in a range of 9-15. In certain embodiments, the mean degree of polymerization of all oligosaccharides is in a range of 10.5-14. In some embodiments, the mean degree of polymerization of all oligosaccharides is in a range of 7-15, 7-12, 7-10, 7-8, 9-10, 10-11, 11-12, 11-15, 12-13, 12-14 13-14, 14-15, 15-16, 17-18, 15-20, 3-8, 4-7, or 5-6.

In certain embodiments, the weight percent of dextrose monomer, galactose monomer, and mannose monomer in the oligosaccharide composition is in a range of 10-18. In certain embodiments, the weight percent of dextrose monomer, galactose monomer, and mannose monomer in the oligosaccharide composition is in a range of 11-17. In certain embodiments, the weight percent of dextrose monomer, galactose monomer, and mannose monomer in the oligosaccharide composition is in a range of 12-16. In certain embodiments, the weight percent of dextrose monomer, galactose monomer, and mannose monomer in the oligosaccharide composition is in a range of 13-15.

In some embodiments, the oligosaccharide composition is a mixture of polymers of dextrose, galactose, and mannose in proportions of approximately 45%, 45%, and 10%, by weight respectively. The formula is H—[C₆H₉₋₁₁O₅]ₙ—OH, where the total number of monomer units in a single polymer of the mixture ranges from 2 to approximately 60 (n=2-60), with a mean value for the mixture of approximately 12.6 monomer units. Each monomer unit may be unsubstituted, singly, doubly, or triply substituted with another dextrose, galactose, or mannose unit by any glycosidic isomer.

In some embodiments, the oligosaccharide composition comprises water in a range of 5-75 weight percent. In some embodiments, the oligosaccharide composition comprises water in a range of 25-65 weight percent. In some embodiments, the oligosaccharide composition comprises water in a range of 35-65 weight percent. In some embodiments, the oligosaccharide composition comprises water in a range of 45-55 weight percent.

In some embodiments, the oligosaccharide composition comprises oligosaccharides that have a MWw (g/mol) in a range of 1905-2290. In some embodiments, the oligosaccharide composition comprises oligosaccharides that have a MWw (g/mol) in a range of 1753-2395. In some embodiments, the oligosaccharide composition comprises oligosaccharides that have a MWw (g/mol) in a range of 1750-2400. In some embodiments, the oligosaccharide composition comprises oligosaccharides that have a MWw (g/mol) in a range of 1500-2500. In some embodiments, the oligosaccharide composition comprises oligosaccharides that have a MWw (g/mol) in a range of 1800-2000. In some embodiments, the oligosaccharide composition comprises oligosaccharides that have a MWw (g/mol) in a range of 2000-2300. In some embodiments, the oligosaccharide composition comprises oligosaccharides that have a MWw (g/mol) in a range of 1515-2630. In some embodiments, the oligosaccharide composition comprises oligosaccharides that have a MWw (g/mol) in a range of 1500-2500. In some embodiments, the oligosaccharide composition comprises oligosaccharides that have a MWw (g/mol) in a range of 1740-2400. In some embodiments, the oligosaccharide composition comprises oligosaccharides that have a MWw (g/mol) in a range of 1700-2300. In some embodiments, the oligosaccharide composition comprises oligosaccharides that have a MWw (g/mol) in a range of 1800-1900, 1900-2000, 2000-2100, 2100-2200, 2200-2300, 2300-2400, or 2400-2500.

In some embodiments, the oligosaccharide composition comprises oligosaccharides that have a MWn (g/mol) in a range of 1030-1095. In some embodiments, the oligosaccharide composition comprises oligosaccharides that have a MWn (g/mol) in a range of 981-1214. In some embodiments, the oligosaccharide composition comprises oligosaccharides that have a MWn (g/mol) in a range of 980-1220 In some embodiments, the oligosaccharide composition comprises oligosaccharides that have a MWn (g/mol) in a range of 1000-1050. In some embodiments, the oligosaccharide composition comprises oligosaccharides that have a MWn (g/mol) in a range of 1050-1100. In some embodiments, the oligosaccharide composition comprises oligosaccharides that have a MWn (g/mol) in a range of 890-1300. In some embodiments, the oligosaccharide composition comprises oligosaccharides that have a MWn (g/mol) in a range of 975-1155. In some embodiments, the oligosaccharide composition comprises oligosaccharides that have a MWn (g/mol) in a range of 875-1180. In some embodiments, the oligosaccharide composition comprises oligosaccharides that have a MWn (g/mol) in a range of 940-1120. In some embodiments, the oligosaccharide composition comprises oligosaccharides that have a MWn (g/mol) in a range of 900-950, 950-1000, 1000-1050, 1050-1100, 1100-1150, 1150-1200, or 1200-1250.

In some embodiments, a solution comprising the oligosaccharide composition has a pH in a range of 1.50-6.00. In some embodiments, a solution comprising the oligosaccharide composition has a pH in a range of 1.50-5.00. In some embodiments, a solution comprising the oligosaccharide composition has a pH in a range of 2.00-4.00. In some embodiments, a solution comprising the oligosaccharide composition has a pH in a range of 2.50-3.50.

In some embodiments, the oligosaccharide composition comprises oligosaccharides that have a degree of branching in a range of about 8.5% to about 32%. In some embodiments, the oligosaccharide composition comprises oligosaccharides that have a degree of branching in a range of about 10% to about 35%. In some embodiments, the oligosaccharide composition comprises oligosaccharides that have a degree of branching in a range of about 13% to about 29%. In some embodiments, the oligosaccharide composition comprises oligosaccharides that have a degree of branching in a range of 5-50%, 5-40%, 5-30%, 5-20%, 5-15%, 10-50%, 10-40%, 10-30%, 10-25%, 15-30%, or 15-20%.

In some embodiments, the oligosaccharide composition comprises oligomers having two or more repeat units (DP2+) in a range of 80-100 weight percent. In some embodiments, the oligosaccharide composition comprises oligomers having two or more repeat units (DP2+) in a range of 86-96 weight percent. In some embodiments, the oligosaccharide composition comprises oligomers having two or more repeat units (DP2+) in a range of 86-91 weight percent. In some embodiments, the oligosaccharide composition comprises oligomers having two or more repeat units (DP2+) in a range of 91-96 weight percent. In some embodiments, the oligosaccharide composition comprises oligomers having two or more repeat units (DP2+) in a range of 81-100 weight percent. In some embodiments, the oligosaccharide composition comprises oligomers having two or more repeat units (DP2+) in a range of 80-94 weight percent. In some embodiments, the oligosaccharide composition comprises oligomers having two or more repeat units (DP2+) in a range of 91-96 weight percent. In some embodiments, the oligosaccharide composition comprises oligomers having two or more repeat units (DP2+) in a range of 80-85, 85-87, 86-88, 87-90, 88-91, 89-92, 90-93, 91-94, 92-95, 93-96, or 95-98 weight percent.

In some embodiments, the oligosaccharide composition has a polydispersity index (PDI) of 1.8-2.0. In some embodiments, the oligosaccharide composition has a polydispersity index (PDI) of 1.8-2.1. In some embodiments, the oligosaccharide composition has a PDI of 1.0-1.2, 1.2-1.3, 1.3-1.4, 1.4-1.5, 1.5-1.6, 1.7-1.8, 1.8-2.0, 2.0-2.2, 2.2-2.4, or 2.4-2.6. In some embodiments, the oligosaccharide composition has a PDI of about 1.6, about 1.7, about 1.8, about 1.9, about 2.0, about 2.1, or about 2.2.

In some embodiments, the MWw, MWn, PDI, monomer content (DPI) and/or DP2+ values of oligosaccharides in an oligosaccharide composition are determined using the size exclusion chromatography method described in Example 15.

In some embodiments, the degree of polymerization (DP1-DP7) of oligosaccharides in an oligosaccharide composition are determined using the size exclusion chromatography method described in Example 17.

In some embodiments, the oligosaccharide composition comprises oligomers having at least three linked monomer units (DP3+) in a range of 80-95 weight percent. In some embodiments, the oligosaccharide composition comprises oligomers having at least three linked monomer units (DP3+) in a range of 85-90 weight percent. In some embodiments, the oligosaccharide composition comprises oligomers having at least three linked monomer units (DP3+) in a range of 80-85, 85-87, 86-88, 87-90, 88-91, 89-92, 90-93, 91-94, or 92-95 weight percent.

In some embodiments, the oligosaccharide composition comprises 4.20% to 6.28% monomer (DP1). In some embodiments, the oligosaccharide composition comprises 4% to 5%, 5% to 6%, or 6% to 7% monomer (DPI). In some embodiments, the oligosaccharide composition comprises 6.20% to 8.83% disaccharide (DP2). In some embodiments, the oligosaccharide composition comprises 6% to 6.5%, 6.5% to 7%, 7.5% to 8%, 8% to 8.5%, or 8.5% to 9% disaccharide (DP2). In some embodiments, the oligosaccharide composition comprises 84.91% to 89.58% oligomers having at least three linked monomer units (DP3+). In some embodiments, the oligosaccharide composition comprises 84% to 85%, 85% to 86%, 86% to 87%, 87% to 88%, or 88% to 90% oligomers having at least three linked monomer units (DP3+).

In some embodiments, the oligosaccharide composition comprises less than 0.10% total impurities (excluding monomer). In some embodiments, the oligosaccharide composition comprises less than 0.05% total impurities (excluding monomer). In some embodiments, the oligosaccharide composition comprises less than 0.20%, 0.15%, 0.10%, or 0.05% total impurities (excluding monomer). In some embodiments, the oligosaccharide composition comprises less than 0.10% w/w levoglucosan, less than 0.10% w/w glucuronic acid, less than 0.10% w/w lactic acid, less than 0.10% w/w formic acid, less than 0.10% w/w acetic acid, and less than 0.10% w/w hydroxymethylfurfural (HMF). In some embodiments, the oligosaccharide composition comprises 0.35% w/w levoglucosan, 0.03% w/w lactic acid, and/or 0.06% w/w formic acid. In some embodiments, the oligosaccharide composition comprises 0.28-0.43% w/w levoglucosan, 0.00-0.03% w/w lactic acid, and/or 0.05-0.07% w/w formic acid.

In some embodiments, the oligosaccharide composition comprises a MWw of 1753-2395, a MWn of 981-1214, and/or a PDI of 1.8-2.0.

The oligosaccharide compositions described herein, and prepared according to the methods described herein, can be characterized and distinguished from prior art compositions using permethylation analysis. See, e.g., Zhao, Y., et al. ‘Rapid, sensitive structure analysis of oligosaccharides,’ PNAS March 4, 1997 94 (5) 1629-1633; Kailemia, M. J., et al. ‘Oligosaccharide analysis by mass spectrometry: A review of recent developments,’ Anal Chem. 2014 Jan. 7; 86(1): 196-212. Accordingly, in another aspect, oligosaccharide compositions are provided herein that comprise a plurality of oligosaccharides that are minimally digestible in humans, the plurality of oligosaccharides comprising monomer radicals. The molar percentages of different types of monomer radicals in the plurality of oligosaccharides can be quantified using a permethylation assay as described in Example 13. The permethylation assay is performed on a de-monomerized sample of the composition.

In some embodiments, the plurality of oligosaccharides comprises two or more monomer radicals selected from radicals (1)-(40):
(1) t-mannopyranose monoradicals, representing 3.0-4.1 mol % of monomer radicals in the plurality of oligosaccharides;
(2) t-glucopyranose monoradicals, representing 11.4-16.3 mol % of monomer radicals in the plurality of oligosaccharides;
(3) t-galactofuranose monoradicals, representing 1.3-7.8 mol % of monomer radicals in the plurality of oligosaccharides;
(4) t-glucofuranose monoradicals, representing 0-1.4 mol % of monomer radicals in the plurality of oligosaccharides;
(5) t-galactopyranose monoradicals, representing 8.3-12.5 mol % of monomer radicals in the plurality of oligosaccharides;
(6) 3-glucopyranose monoradicals, representing 3.0-4.9 mol % of monomer radicals in the plurality of oligosaccharides;
(7) 2-mannopyranose and/or 3-mannopyranose monoradicals, representing 1.2-1.9 mol % of monomer radicals in the plurality of oligosaccharides;
(8) 2-glucopyranose monoradicals, representing 2.4-3.2 mol % of monomer radicals in the plurality of oligosaccharides;
(9) 2-galactofuranose and/or 2-glucofuranose monoradicals, representing 0.9-2.3 mol % of monomer radicals in the plurality of oligosaccharides;
(10) 3-galactopyranose monoradicals, representing 2.9-3.9 mol % of monomer radicals in the plurality of oligosaccharides;
(11) 4-mannopyranose and/or 5-mannofuranose and/or 3-galactofuranose monoradicals, representing 1.7-2.9 mol % of monomer radicals in the plurality of oligosaccharides;
(12) 6-mannopyranose monoradicals, representing 2.0-2.9 mol % of monomer radicals in the plurality of oligosaccharides;
(13) 2-galactopyranose monoradicals, representing 1.8-2.7 mol % of monomer radicals in the plurality of oligosaccharides;
(14) 6-glucopyranose monoradicals, representing 7.6-10.8 mol % of monomer radicals in the plurality of oligosaccharides;
(15) 4-galactopyranose and/or 5-galactofuranose monoradicals, representing 2.6-3.8 mol % of monomer radicals in the plurality of oligosaccharides;
(16) 4-glucopyranose and/or 5-glucofuranose and/or 6-mannofuranose monoradicals, representing 3.0-4.5 mol % of monomer radicals in the plurality of oligosaccharides;
(17) 6-glucofuranose monoradicals, representing 0-1.6 mol % of monomer radicals in the plurality of oligosaccharides;
(18) 6-galactofuranose monoradicals, representing 1.4-5.0 mol % of monomer radicals in the plurality of oligosaccharides;
(19) 6-galactopyranose monoradicals, representing 5.8-9.1 mol % of monomer radicals in the plurality of oligosaccharides;
(20) 3,4-galactopyranose and/or 3,5-galactofuranose and/or /2,3-galactopyranose diradicals, representing 0.9-1.4 mol % of monomer radicals in the plurality of oligosaccharides;
(21) 3,4-glucopyranose and/or 3,5-glucofuranose diradicals, representing 0-1.1 mol % of monomer radicals in the plurality of oligosaccharides;
(22) 2,4-glucopyranose and/or 2,5-glucofuranose and/or 2,4-galactopyranose and/or 2,5-galactofuranose diradicals, representing 0.9-1.4 mol % of monomer radicals in the plurality of oligosaccharides;
(23) 4,6-mannopyranose and/or 5,6-mannofuranose diradicals, representing 0.5-0.7 mol % of monomer radicals in the plurality of oligosaccharides;
(24) 3,6-mannofuranose diradicals, representing 0-0.1 mol % of monomer radicals in the plurality of oligosaccharides;
(25) 3,6-glucopyranose diradicals, representing 1.4-2.8 mol % of monomer radicals in the plurality of oligosaccharides;
(26) 3,6-mannopyranose and/or 2,6-mannofuranose diradicals, representing 0.4-0.7 mol % of monomer radicals in the plurality of oligosaccharides;
(27) 2,6-mannopyranose diradicals, representing 0.3-0.5 mol % of monomer radicals in the plurality of oligosaccharides;
(28) 3,6-glucofuranose diradicals, representing 0.1-0.4 mol % of monomer radicals in the plurality of oligosaccharides;
(29) 2,6-glucopyranose and/or 4,6-glucopyranose and/or 5,6-glucofuranose diradicals, representing 1.1-3.6 mol % of monomer radicals in the plurality of oligosaccharides;
(30) 3,6-galactofuranose diradicals, representing 0.9-1.4 mol % of monomer radicals in the plurality of oligosaccharides;
(31) 4,6-galactopyranose and/or 5,6-galactofuranose diradicals, representing 2.1-2.9 mol % of monomer radicals in the plurality of oligosaccharides;
(32) 3,6-galactopyranose and/or 2,6-galactofuranose diradicals, representing 1.6-3.0 mol % of monomer radicals in the plurality of oligosaccharides;
(33) 2,6-galactopyranose diradicals, representing 0.7-1.6 mol % of monomer radicals in the plurality of oligosaccharides;
(34) 3,4,6-mannopyranose and/or 3,5,6-mannofuranose and/or 2,3,6-mannofuranose triradicals, representing 0-0.3 mol % of monomer radicals in the plurality of oligosaccharides;
(35) 3,4,6-galactopyranose and/or 3,5,6-galactofuranose and/or 2,3,6-galactofuranose triradicals, representing 0.5-1.1 mol % of monomer radicals in the plurality of oligosaccharides;
(36) 3,4,6-glucopyranose and/or 3,5,6-glucofuranose triradicals, representing 0.2-0.5 mol % of monomer radicals in the plurality of oligosaccharides;
(37) 2,3,6-mannopyranose and/or 2,4,6-mannopyranose and/or 2,5,6-mannofuranose triradicals, representing 0-0.5 mol % of monomer radicals in the plurality of oligosaccharides;
(38) 2,4,6-glucopyranose and/or 2,5,6-glucofuranose triradicals, representing 0-1.4 mol % of monomer radicals in the plurality of oligosaccharides;
(39) 2,3,6-galactopyranose and/or 2,4,6-galactopyranose and/or 2,5,6-galactofuranose triradicals, representing 0.4-0.9 mol % of monomer radicals in the plurality of oligosaccharides; and
(40) 2,3,6-glucopyranose triradicals, representing 0.1-0.5 mol % of monomer radicals in the plurality of oligosaccharides.

In some embodiments, about 8-30% of the total glycosidic bonds in an oligosaccharide composition are 1,2 glycosidic bonds. In some embodiments, about 10.5-25% of the total glycosidic bonds in an oligosaccharide composition are 1,2 glycosidic bonds. In some embodiments, about 9.5-32% of the total glycosidic bonds in an oligosaccharide composition are 1,2 glycosidic bonds. In some embodiments, about 13-27% of the total glycosidic bonds in an oligosaccharide composition are 1,2 glycosidic bonds. In some embodiments, 5-50%, 5-40%, 5-30%, 5-20%, 5-15%, 10-50%, 10-40%, 10-30%, 10-25%, 15-30%, or 15-20% of the total glycosidic bonds in an oligosaccharide composition are 1,2 glycosidic bonds.

In some embodiments, about 14.5-34% of the total glycosidic bonds in an oligosaccharide composition are 1,3 glycosidic bonds. In some embodiments, about 17-30% of the total glycosidic bonds in an oligosaccharide composition are 1,3 glycosidic bonds. In some embodiments, about 9.5-27% of the total glycosidic bonds in an oligosaccharide composition are 1,3 glycosidic bonds. In some embodiments, about 12.5-23.5% of the total glycosidic bonds in an oligosaccharide composition are 1,3 glycosidic bonds. In some embodiments, 5-50%, 10-40%, 10-30%, 10-20%, 5-15%, 10-50%, 10-40%, 10-30%, 10-25%, 15-30%, or 15-20% of the total glycosidic bonds in an oligosaccharide composition are 1,3 glycosidic bonds.

In some embodiments, about 10-26% of the total glycosidic bonds in an oligosaccharide composition are 1,4 glycosidic bonds. In some embodiments, about 12-22% of the total glycosidic bonds in an oligosaccharide composition are 1,4 glycosidic bonds. In some embodiments, about 10-29.5% of the total glycosidic bonds in an oligosaccharide composition are 1,4 glycosidic bonds. In some embodiments, about 13-25% of the total glycosidic bonds in an oligosaccharide composition are 1,4 glycosidic bonds. In some embodiments, 5-50%, 10-40%, 10-30%, 10-20%, 5-15%, 10-50%, 10-40%, 10-30%, 10-25%, 15-30%, or 15-20% of the total glycosidic bonds in an oligosaccharide composition are 1,4 glycosidic bonds.

In some embodiments, about 32-57% of the total glycosidic bonds in an oligosaccharide composition are 1,6 glycosidic bonds. In some embodiments, about 35-52% of the total glycosidic bonds in an oligosaccharide composition are 1,6 glycosidic bonds. In some embodiments, about 23-65% of the total glycosidic bonds in an oligosaccharide composition are 1,6 glycosidic bonds. In some embodiments, about 30-56% of the total glycosidic bonds in an oligosaccharide composition are 1,6 glycosidic bonds. In some embodiments, 15-70%, 20-60%, 20-40%, 25-50%, 30-50%, 30-40%, or 30-60% of the total glycosidic bonds in an oligosaccharide composition are 1,6 glycosidic bonds.

In some embodiments, an oligosaccharide composition comprises 17.5-43% total furanose. In some embodiments, an oligosaccharide composition comprises 20.5-37% total furanose. In some embodiments, an oligosaccharide composition comprises 14-60% total furanose. In some embodiments, an oligosaccharide composition comprises 20.5-50% total furanose. In some embodiments, an oligosaccharide composition comprises 10-60%, 10-50%, 15-40%, 20-40%, 20-30%, or 30-50% total furanose.

In some embodiments, the oligosaccharide composition comprises at least one glucofuranose or glucopyranose radical, at least one mannofuranose or mannopyranose radical, and at least one galactofuranose or galactopyranose radical.

In some embodiments, an oligosaccharide composition is provided, comprising a plurality of oligosaccharides comprising monomer radicals (1)-(40) in the molar percentages shown in Table 2.

TABLE 2
Permethylation Data

| | Mean mol | | Mean mol |
|---|---|---|---|
| | % +3 | Mean mol | % −3 |
| Radicals | STD | % | STD |
| t-mannopyranose | 4.10% | 3.56% | 3.02% |
| t-glucopyranose | 16.33% | 13.89% | 11.44% |
| t-galactofuranose | 7.78% | 4.52% | 1.26% |
| t-glucofuranose | 1.38% | 0.64% | 0.00% |
| t-galactopyranose | 12.48% | 10.38% | 8.29% |
| 3-glucopyranose | 4.88% | 3.95% | 3.02% |
| 2-mannopyranose and/or | 1.94% | 1.57% | 1.20% |
| 3-mannopyranose | | | |
| 2-glucopyranose | 3.22% | 2.83% | 2.44% |
| 2-galactofuranose and/or | 2.32% | 1.62% | 0.93% |
| 2-glucofuranose | | | |
| 3-galactopyranose | 3.92% | 3.43% | 2.94% |
| 4-mannopyranose and/or | 2.93% | 2.34% | 1.75% |
| 5-mannofuranose and/or | | | |
| 3-galactofuranose | | | |
| 6-mannopyranose | 2.87% | 2.44% | 2.01% |
| 2-galactopyranose | 2.71% | 2.28% | 1.85% |
| 6-glucopyranose | 10.78% | 9.22% | 7.66% |
| 4-galactopyranose and/or | 3.80% | 3.22% | 2.65% |
| 5-galactofuranose | | | |
| 4-glucopyranose and/or | 4.25% | 3.66% | 3.06% |
| 5-glucofuranose and/or | | | |
| 6-mannofuranose | | | |
| 6-glucofuranose | 1.55% | 0.81% | 0.08% |
| 6-galactofuranose | 4.96% | 3.19% | 1.42% |
| 6-galactopyranose | 9.06% | 7.44% | 5.81% |
| 3,4-galactopyranose and/or | 1.42% | 1.16% | 0.90% |
| 3,5-galactofuranose and/or | | | |
| 2,3-galactopyranose | | | |
| 3,4-glucopyranose and/or | 1.04% | 0.43% | 0.00% |
| 3,5-glucofuranose | | | |
| 2,4-glucopyranose and/or | 1.39% | 1.16% | 0.92% |
| 2,5-glucofuranose and/or | | | |
| 2,4-galactopyranose and/or | | | |
| 2,5-galactofuranose | | | |
| 4,6-mannopyranose and/or | 0.69% | 0.59% | 0.49% |
| 5,6-mannofuranose | | | |
| 3,6-mannofuranose | 0.11% | 0.02% | 0.00% |
| 3,6-glucopyranose | 2.80% | 2.10% | 1.40% |
| 3,6-mannopyranose and/or | 0.67% | 0.53% | 0.39% |
| 2,6-mannofuranose | | | |
| 2,6-mannopyranose | 0.54% | 0.41% | 0.28% |
| 3,6-glucofuranose | 0.39% | 0.27% | 0.16% |
| 2,6-glucopyranose and/or | 3.58% | 2.33% | 1.08% |
| 4,6-glucopyranose and/or | | | |
| 5,6-glucofuranose | | | |
| 3,6-galactofuranose | 1.37% | 1.15% | 0.93% |
| 4,6-galactopyranose and/or | 2.86% | 2.48% | 2.11% |
| 5,6-galactofuranose | | | |
| 3,6-galactopyranose and/or | 2.98% | 2.28% | 1.58% |
| 2,6-galactofuranose | | | |
| 2,6-galactopyranose | 1.62% | 1.15% | 0.68% |
| 3,4,6-mannopyranose and/or | 0.30% | 0.07% | 0.00% |
| 3,5,6-mannofuranose and/or | | | |
| 2,3,6-mannofuranose | | | |
| 3,4,6-galactopyranose and/or | 1.11% | 0.82% | 0.53% |
| 3,5,6-galactofuranose and/or | | | |
| 2,3,6-galactofuranose | | | |
| 3,4,6-glucopyranose and/or | 0.47% | 0.35% | 0.22% |
| 3,5,6-glucofuranose | | | |
| 2,3,6-mannopyranose and/or | 0.49% | 0.17% | 0.00% |
| 2,4,6-mannopyranose and/or | | | |
| 2,5,6-mannofuranose | | | |
| 2,4,6-glucopyranose and/or | 1.36% | 0.56% | 0.00% |
| 2,5,6-glucofuranose | | | |
| 2,3,6-galactopyranose and/or | 0.91% | 0.66% | 0.41% |
| 2,4,6-galactopyranose and/or | | | |
| 2,5,6-galactofuranose | | | |
| 2,3,6-glucopyranose | 0.48% | 0.31% | 0.13% |

In certain embodiments, the oligosaccharide compositions are free from monomer. In other embodiments, the oligosaccharide compositions comprise monomer.

The oligosaccharide compositions described herein, and prepared according to the methods described herein, can be characterized and distinguished from prior art compositions using two-dimensional heteronuclear NMR. Accordingly, in another aspect, oligosaccharide compositions are provided that comprise a plurality of oligosaccharides that are minimally digestible in humans, the compositions being characterized by a heteronuclear single quantum correlation (HSQC) NMR spectrum comprising signals 5, 6, 7, and 15, each signal having a center position and an area:

| | Center Position | | |
|---|---|---|---|
| | (ppm) | | Area under the curve (AUC) |
| Signal | ¹H | ¹³C | (% of total areas of all signals) |
| 5 | 3.96 | 70.62 | 9.28-10.71 |
| 6 | 3.92 | 71.26 | 1.52-2.03 |
| 7 | 3.55 | 71.34 | 3.40-6.13 |
| 15 | 4.44 | 103.86 | 1.84-2.44 |

In some embodiments, the spectrum further comprises 1-2 (e.g., one or two) signals selected from signals 10 and 14, and defined as follows:

| | Center Position | | |
|---|---|---|---|
| | (ppm) | | Area under the curve (AUC) |
| Signal | ^{l}H | ¹³C | (% of total areas of all signals) |
| 10 | 3.33 | 73.74 | 10.21-12.09 |
| 14 | 4.5 | 103.29 | 5.03-6.41 |

In some embodiments, the spectrum further comprises 1-3 (e.g., one, two, or three) signals selected from signals 11, 12, and 13, and defined as follows:

| | Center Position | | |
|---|---|---|---|
| | (ppm) | | Area under the curve (AUC) |
| Signal | ¹H | ¹³C | (% of total areas of all signals) |
| 11 | 4.06 | 77.34 | 3.68-4.50 |
| 12 | 4.11 | 81.59 | 3.10-3.82 |
| 13 | 4.96 | 98.7 | 10.65-12.31 |

In some embodiments, the spectrum comprises 1-3 (e.g., one, two, or three) signals selected from signals 11, 12, and 13, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, the spectrum comprises 1-15 (e.g., one, two, or three) signals selected from signals 1-15, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 18.59-27.53 |
| 2 | 3.75 | 66.06 | 2.79-7.27 |
| 3 | 3.97 | 66.15 | 1.82-3.8 |
| 4 | 3.96 | 69.28 | 0.71-4.47 |
| 5 | 3.96 | 70.62 | 8.81-11.19 |
| 6 | 3.92 | 71.26 | 1.35-2.2 |
| 7 | 3.55 | 71.34 | 2.48-7.04 |
| 8 | 3.97 | 71.56 | 3.06-4.74 |
| 9 | 3.72 | 72.35 | 4.16-11.64 |
| 10 | 3.33 | 73.74 | 9.58-12.72 |
| 11 | 4.06 | 77.34 | 3.4-4.78 |
| 12 | 4.11 | 81.59 | 2.86-4.06 |
| 13 | 4.96 | 98.7 | 10.09-12.87 |
| 14 | 4.5 | 103.29 | 4.57-6.87 |
| 15 | 4.44 | 103.86 | 1.64-2.64 |

In some embodiments, the spectrum comprises 1-15 (e.g., one, two, or three) signals selected from signals 1-15, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.18-27.11 |
| 2 | 3.75 | 66.06 | 3.31-6.1 |
| 3 | 3.97 | 66.15 | 2.37-3.69 |
| 4 | 3.96 | 69.28 | 0.42-4.72 |
| 5 | 3.96 | 70.62 | 8.43-11.69 |
| 6 | 3.92 | 71.26 | 1.09-3.1 |
| 7 | 3.55 | 71.34 | 4.01-6.37 |
| 8 | 3.97 | 71.56 | 2.77-4.29 |
| 9 | 3.72 | 72.35 | 6.28-9.25 |
| 10 | 3.33 | 73.74 | 10.48-12 |
| 11 | 4.06 | 77.34 | 3.04-4.22 |
| 12 | 4.11 | 81.59 | 2.63-3.57 |
| 13 | 4.96 | 98.7 | 9.9-13.39 |
| 14 | 4.5 | 103.29 | 4.45-6.7 |
| 15 | 4.44 | 103.86 | 1.6-2.63 |

In some embodiments, the spectrum comprises 1-15 (e.g., one, two, or three) signals selected from signals 1-15, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, signals 5, 6, 7, 15, 10, 14, 11, 12, and 13 are each further characterized by an ¹H integral region and a ¹³C integral region, defined as follows:

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | to |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 15 | 4.44 | 4.41 | 4.46 | 103.86 | 103.56 | 104.15 |
| 10 | 3.33 | 3.27 | 3.4 | 73.74 | 73.26 | 74.22 |
| 14 | 4.5 | 4.47 | 4.54 | 103.29 | 102.87 | 103.70 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |

In some embodiments, signals 1-15 are each characterized by an ¹H integral region and a ¹³C integral region, defined as follows:

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | to |
| 1 | 3.68 | 3.61 | 3.75 | 63.42 | 62.64 | 64.20 |
| 2 | 3.75 | 3.72 | 3.78 | 66.06 | 65.50 | 66.62 |
| 3 | 3.97 | 3.94 | 4.00 | 66.15 | 65.81 | 66.49 |
| 4 | 3.96 | 3.94 | 3.98 | 69.28 | 69.04 | 69.52 |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 8 | 3.97 | 3.94 | 4.00 | 71.56 | 71.29 | 71.84 |
| 9 | 3.72 | 3.67 | 3.77 | 72.35 | 71.95 | 72.74 |
| 15 | 4.44 | 4.41 | 4.46 | 103.86 | 103.56 | 104.15 |
| 10 | 3.33 | 3.27 | 3.4 | 73.74 | 73.26 | 74.22 |
| 14 | 4.5 | 4.47 | 4.54 | 103.29 | 102.87 | 103.70 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |

In certain embodiments, the NMR spectrum is obtained by subjecting a sample of the composition to HSQC NMR, wherein the sample is a solution in a deuterated solvent. Suitable deuterated solvents in include deuterated acetonitrile, deuterated acetone, deuterated methanol, D₂O, and mixtures thereof. In a particular embodiment, the deuterated solvent is D₂O. In certain embodiments, the NMR spectrum is obtained using the conditions described in Example 14.

Exemplary oligosaccharide compositions may be prepared according to the procedures described herein.

### III. Methods of Use

As described herein, oligosaccharide compositions may be used to reduce pathogen (e.g., CRE or VRE) levels and/or pathogen colonization in subjects with elevated pathogen levels. In some embodiments, oligosaccharide compositions may be used to increase levels of commensal bacteria in subjects.

In some embodiments, the selected oligosaccharide composition is useful for controlling (e.g. reducing) pathogen levels. In some embodiments, the selected oligosaccharide composition is useful for controlling (e.g. reducing) pathogen levels relative to commensals (e.g., non-pathogenic commensals). In some embodiments, the selected oligosaccharide composition is useful for controlling (e.g. reducing) absolute pathogen levels in a subject.

In some embodiments, commensal bacteria refer to bacteria commonly associated with a healthy state of a microbiome in a particular niche, e.g., the gastrointestinal tract (e.g., the intestines), and/or are generally considered non-pathogenic.

In some embodiments, the selected oligosaccharide composition is useful for controlling (e.g. reducing) pathobiont levels. In some embodiments, the selected oligosaccharide composition is useful for controlling (e.g. reducing) pathobiont levels relative to commensals (e.g., non-pathogenic commensals). In some embodiments, the selected oligosaccharide composition is useful for controlling (e.g. reducing) absolute pathobiont levels in a subject. In some embodiments, pathobionts include bacteria (and fungi) that are potentially pathological (disease-causing), though, under normal circumstances, non-pathologically co-exist with the subject, e.g., as a non-harming symbiont. In some embodiments, dysbiosis causes a non-pathological bacterium to become pathological. In some embodiments, pathobionts are as described in Hornef, M. “Pathogens, Commensal Symbionts, and Pathobionts: Discovery and Functional Effects on the Host”, ILAR Journal, Volume 56, Issue 2, 2015, Pages 159-162.

In some embodiments, the selected oligosaccharide composition is useful for controlling relative levels of pathogens and commensals. In some embodiments, the selected oligosaccharide composition is useful for controlling relative levels of pathobionts and commensals. In some embodiments, the selected oligosaccharide composition is useful for controlling relative levels of pathogenic commensals and non-pathogenic commensals.

As described herein, oligosaccharide compositions may be used to affect the structure (e.g., composition) and/or function (e.g. metabolic activity) of the gut microbiota. In some embodiments, the selected oligosaccharide compositions confer beneficial health effects on a subject. Subjects that may benefit from the methods and uses described herein (e.g., uses of the oligosaccharide compositions to treat subjects (e.g., treat infections) and uses of the oligosaccharide compositions to reduce the abundance of pathogens or pathobionts), include immunocompromised or immunosuppressed subjects. Subjects that may benefit from the methods and uses described herein include subjects undergoing transplant procedures, e.g., hematopoietic stem cell transplantation (HSCT) or solid organ transplant, or other medical procedures, e.g., surgery, e.g. of the gastrointestinal tract. Subjects that may benefit from the methods and uses described herein include other immunocompromised subjects, e.g., subjects with hematological malignancies or cirrhosis (e.g., liver cirrhosis). Subjects that may benefit from the methods and uses described herein include subjects admitted to intensive care units.

In some embodiments, the selected oligosaccharide compositions described herein reduce the abundance (e.g., relative abundance or absolute abundance) of pathogens or pathobionts (e.g., in the gastrointestinal tract), e.g., when compared to a baseline (e.g., untreated (population of) subject(s), or a subject prior to treatment). In some embodiments, the selected oligosaccharide compositions described herein promote growth of commensal bacteria over growth of pathogens or pathobionts (e.g., in the gastrointestinal tract, e.g., the intestines, e.g., the large intestine or colon). In some embodiments, subjects achieve decolonization with MDR pathogens (e.g., vancomycin-resistant Enterococcus (VRE), extended-spectrum beta lactamase-producing Enterobacteriaceae (ESBLE), and carbapenem-resistant Enterobacteriaceae (CRE), e.g., levels of these bacteria are near to or fall below detectable levels. The reduction in the abundance (e.g., relative abundance or absolute abundance) of a pathogen or pathobiont, may be determined, e.g., by subjecting a sample (e.g., a stool sample) from a subject to nucleic acid sequencing (e.g., whole genome sequencing) and other assays (e.g., colony-forming units (cfu)/g feces by culture). In some embodiments, the selected oligosaccharide compositions described herein promote an increase in alpha-diversity (e.g. an increase in bacterial taxa diversity, e.g., as determined by measuring Shannon diversity, e.g. by nucleic acid sequencing). In some embodiments, the selected oligosaccharide compositions described herein promote richness of the bacterial community. In some embodiments, the selected oligosaccharide compositions described herein reduce inflammation, e.g. inflammation associated with pathogens or pathobionts or other bacteria. The reduction may be determined by measuring one or more markers of inflammation, e.g. IFN-γ, IL-1β, IL-2, IL-4, IL-6, IL-8, IL-10, IL-12p70, IL-13, and TNF-α. The markers can be determined, e.g., from stool or blood samples. In some embodiments, the selected oligosaccharide compositions described herein treat infections, e.g., bacterial infections or fungal infections. In some embodiments, the selected oligosaccharide compositions described herein reduce infections (e.g., the rate of infections), including secondary or opportunistic infections (e.g., hospital acquired infections (HAI)), including, e.g., central line-associated bloodstream infections (CLABSI), catheter-associated urinary tract Infection (CAUTI), and C. difficile infections (CDI)). In some embodiments, the selected oligosaccharide compositions described herein reduce the rate of hospitalizations, e.g., due to or caused by infections. In some embodiments, the selected oligosaccharide compositions described herein shorten the time period of hospitalization required, e.g., to treat or resolve the infections.

In some embodiments, the selected oligosaccharide compositions described herein is administered to a subject having a high likelihood of developing an infection, e.g., to prevent the infection or slow the progression of an infection. In some embodiments, treatment with the selected oligosaccharide compositions described herein is provided until the subject's infection is resolved or the subject is at a low risk of acquiring an infection, or is at a low risk of acquiring a re-infection.

In some embodiments, the selected oligosaccharide compositions described herein is administered to a subject having a high likelihood of developing a rejection of a transplant, e.g., graft-versus-host disease (GvHD), e.g., to prevent the rejection or slow the progression of the rejection, e.g., at a time after the transplant is received by the subject. In some embodiments, treatment with the selected oligosaccharide compositions described herein is provided until the subject's transplant rejection reaction is resolved or the subject is at a low risk of rejecting the transplant.

In some embodiments, the selected oligosaccharide compositions described herein can be provided with standard-of-care treatment (e.g., administration of antibiotics). In some embodiments, the selected oligosaccharide compositions described herein can be provided without standard-of-care treatment (e.g., administration of antibiotics).

In some embodiments, an oligosaccharide composition described herein can be used to benefit (e.g., treat) patients having detectable commensal bacteria in the gut, e.g., patients with gut microbiota that are not devoid of detectable commensal bacteria. In some embodiments, an oligosaccharide composition described herein is administered to a patient with low levels of commensal bacteria, e.g., a patient with gut microbiota that is not devoid of commensal bacteria or a patient with a gut microbiota that has not been completely depleted (e.g., resulting from use of antibiotics or chemotherapy), e.g., for treatment purposes.

In some embodiments, the oligosaccharide composition is formulated as powder, e.g., for reconstitution (e.g., in water) for oral administration. In some embodiments, the oligosaccharide composition is formulated as a pharmaceutical composition for delivery by a feeding tube. In some embodiments, the oligosaccharide composition is formulated as a pharmaceutical composition for delivery by total parenteral nutrition (TPN).

The oligosaccharide composition may be administered to the subject on a daily, weekly, biweekly, or monthly basis. In some embodiments, the composition is administered to the subject more than once per day (e.g., 2, 3, or 4 times per day). In some embodiments, the composition is administered to the subject once or twice per day for one, two, three, or four weeks in a row.

In some embodiments, the composition is administered to the subject according to the following schedule: 18 grams total on each of days 1 and 2 of a treatment protocol; 36 grams total on each of days 3 and 4 of a treatment protocol; and 72 grams total on each of days 5-14 of a treatment protocol.

In some embodiments, the composition is administered to the subject according to the following schedule: 18 grams total on each of days 1-7 of a treatment protocol; 36 grams total on each of days 8-14 of a treatment protocol; 54 grams total on each of days 15-21 of a treatment protocol; and 72 grams total on each of days 22-28 of a treatment protocol.

In some embodiments, an effective amount of an oligosaccharide is a total of 5-200 grams, 5-150 grams, 5-100 grams, 5-75 grams, 5-50 grams, 5-25 grams, 10-50 grams, 25-50 grams, 30-60 grams, 50-75 grams, 50-100 grams, 18-72 grams, or 36-72 grams administered daily.

The oligosaccharide composition of the disclosure is well tolerated by a subject (e.g., oligosaccharide compositions do not cause or cause minimal discomfort, e.g., production of gas or gastrointestinal discomfort, in subjects). In some embodiments, 5-200 grams, 5-150 grams, 5-100 grams, 5-75 grams, 5-50 grams, 5-25 grams, 10-50 grams, 25-50 grams, 30-60 grams, 50-75 grams, 50-100 grams, 18-72 grams, or 36-72 grams of total daily dose are well tolerated by a subject. The amount of an oligosaccharide composition that is administered to the subject at a single time or in a single dose is well tolerated by the subject.

In some embodiments, the amount of the oligosaccharide composition that is administered to the subject at a single time or in a single dose is more tolerated by the subject than a similar amount of commercial low-digestible sugars such as fructooligosaccharides (FOS). Commercial low-digestible sugars are known in the art to be poorly tolerated in subjects (See, e.g., Grabitske, H. A., Critical Reviews in Food Science and Nutrition, 49:327-360 (2009)), e.g., at high doses. For example, tolerability studies of FOS indicate that 20 grams FOS per day causes mild gastrointestinal symptoms and that 30 grams FOS per day causes major discomfort and gastrointestinal symptoms.

In some embodiments, oligosaccharide compositions provided herein effectively reduce colonization with, prevent colonization with, or reduce the risk of an adverse effect of a pathogen or pathobiont to a subject. In some embodiments, provided is a method of decolonizing the gastrointestinal tract (e.g., all of the GI tract or part of the GI tract, e.g. the small intestine or the large intestine) from a pathogen, pathobiont or an antibiotic resistance gene carrier. In some embodiments, the method comprises shifting the microbial community in the gastrointestinal tract toward a commensal population, e.g., thereby replacing (e.g. outcompeting) a pathogen or an antibiotic resistance gene carrier.

In some embodiments, the oligosaccharide compositions provided herein are administered to a subject reduce the spread of pathogen to other untreated subjects. In some embodiments, the oligosaccharide composition is administered in an effective amount and/or to a sufficient number of subjects that the spread of the pathogen, e.g., from a first subject to a second subject, is reduced. Such reduction might be measured by any of the methods described herein or any other conceivable method.

In some embodiments, provided is a method of reducing a pathogen or pathobiont reservoir in a subject by administering an oligosaccharide composition to the subject, e.g., in an effective amount and/or to a sufficient number of subjects that the pathogen or pathobiont reservoir is reduced. In some embodiments, the pathogen or pathobiont reservoir is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard. In some embodiments, a pathogen or pathobiont reservoir may represent about 5%, about 10%, about 15%, about 20%, about 30%, about 35%, about 40%, about 45%, about 50%, about 55%, about 60%, about 65%, about 70%, about 75%, about 80%, or about 85% of the total bacterial reservoir of a subject (e.g., about 5%, about 10%, about 15%, about 20%, about 30%, about 35%, about 40%, about 45%, about 50%, about 55%, about 60%, about 65%, about 70%, about 75%, about 80%, or about 85% of the total bacterial population in the gut or intestines of a subject). In some embodiments, the pathogen or pathobiont reservoir comprises the pathogen or pathobiont biomass. In some embodiments, the bacterial reservoir comprises the total bacterial biomass.

In some embodiments, methods described herein comprise administering an oligosaccharide composition to a subject in an effective amount to reduce the total pathogen or pathobiont reservoir from about 80% to about 5% of the total bacterial reservoir, from about 80% to about 10% of the total bacterial reservoir, from about 80% to about 20% of the total bacterial reservoir, from about 80% to about 30% of the total bacterial reservoir, from about 80% to about 40% of the total bacterial reservoir, or from about 80% to about 50% of the total bacterial reservoir. In some embodiments, methods described herein comprise administering an oligosaccharide composition to a subject in an effective amount to reduce the total pathogen or pathobiont reservoir from about 50-80% to about 5% of the total bacterial reservoir, from about 50-80% to about 10% of the total bacterial reservoir, from about 50-80% to about 20% of the total bacterial reservoir, from about 50-80% to about 30% of the total bacterial reservoir, or from about 50-80% to about 40% of the total bacterial reservoir.

In some embodiments, provided is a method of modulating the biomass of a pathogen or pathobiont or an antibiotic resistance gene carrier. In some embodiments, the modulating comprises increasing or decreasing, e.g., the biomass of a pathogen or pathobiont or an antibiotic resistance gene carrier. In some embodiments, the oligosaccharide composition is administered in an effective amount and/or to a sufficient number of subjects, that the reservoir or biomass of a pathogen or pathobiont is reduced. In some embodiments, the oligosaccharide composition is administered in an effective amount that pathogen or pathobiont biomass is modulated, e.g., reduced (e.g., the number of pathogen or pathobionts and/or the number of drug- or antibiotic-resistance gene or MDR element carriers is modulated). In some embodiments, provided is a method of modulating the number of pathogen or pathobionts or antibiotic resistance gene carriers (e.g., in a population, e.g., a microbial population).

Exemplary pathogens include Enterobacteriaciae (e.g., a family comprising Plesiomonas, Shigella, or Salmonella), Clostridium (e.g., a genus comprising Clostridium difficile), Enterococcus, Staphylococcus (e.g., a genus comprising Staphylococcus aureus), Campylobacter, Vibrio, Aeromonas, Norovirus, Astrovirus, Adenovirus, Sapovirus, or Rotavirus.

In some embodiments, the pathogen is a carbapenem-resistant Enterobacteriaceae (CRE). In some embodiments, the pathogen is a vancomycin-resistant Enterococci (VRE). In some embodiments, the pathogen is an extended-spectrum beta-lactamase (ESBL) producing organism.

In some embodiments, the pathogen includes Enterobacteriaciae (e.g., a family comprising Plesiomonas, Shigella, or Salmonella). In some embodiments, the pathogen includes Clostridium (e.g., a genus comprising Clostridium difficile). In some embodiments, the pathogen includes Enterococcus. In some embodiments, the pathogen includes Staphylococcus.

In some embodiments, the method comprises reducing the spread of a pathogen by administering to a subject a oligosaccharide composition, e.g., in an effective amount and/or to a sufficient number of subjects that the spread of the pathogen is reduced. In some embodiments, the spread of a pathogen is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard. In some embodiments, the spread of a pathogen comprises the spread from a first subject to a second subject. In some embodiments, the spread of a pathogen comprises the spread from a first subject or a second subject to an entity which can harbor the pathogen (e.g., another individual or an inanimate object, e.g., facility built surface (e.g. sink, door handle, toilet, faucet) or medical supply (e.g., a package comprising a dressing or device, or a dressing or device itself). In some embodiments, the oligosaccharide composition is administered in an effective amount and/or to a sufficient number of subjects, that the spread of drug- or antibiotic-resistance gene, or a MDR element, e.g., from a first subject to a second subject, is reduced. This reduction might be measured by any of the methods described herein.

In some embodiments, provided is a method of reducing a drug-resistance gene reservoir (e.g., an antibiotic resistance gene reservoir or MDR gene reservoir) in a subject by administering a oligosaccharide composition to the subject, e.g., in an effective amount and/or to a sufficient number of subjects that the drug-resistance gene reservoir (e.g., antibiotic resistance gene reservoir or MDR gene reservoir) is reduced. In some embodiments, a drug-resistance gene reservoir (e.g., an antibiotic resistance gene reservoir or MDR gene reservoir) is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard. Exemplary antibiotic resistance genes include penicillin resistance genes, MecA (conferring methicillin, penicillin and other penicillin-like antibiotic resistance) and other genes that encode the protein PBP2A (penicillin binding protein 2A), carbapenemase resistance genes (e.g., Klebsiella pneumonia carbapenemase (KPC)), betalactamase resistance genes (e.g., New Delhi betalactamase (NDM), OXA, SHV, TIM, CTX-M, VIM), vancomycin resistance genes (e.g., VanA, VanB, vancomycin resistance genes in Enterococcus), AmpC (carbapenem and beta lactam resistance genes in Enterobacteriaceae), fluoroquinoline resistance genes (e.g., Qnr), trimethoprim resistance genes (e.g. dihydrofolate reductase), sulfamethoxazole resistance genes (e.g., dihydropteroate synthetase), ciprofloxacin resistance genes, and aminoglycoside resistance genes (e.g., ribosomal methyltransferase). The reduction of a drug-resistance gene reservoir (e.g., an antibiotic resistance gene reservoir or MDR gene reservoir) may be assessed using any technique described herein, e.g., a technique described for the assessment of a pathogen reservoir.

In some embodiments, provided is a method of reducing the spread of a drug-resistance gene (e.g., an antibiotic resistance gene or MDR gene) comprising administering a oligosaccharide composition to a subject, e.g., in an effective amount and/or to a sufficient number of subjects that the spread of the drug-resistance gene (e.g., antibiotic resistance gene or MDR gene) is reduced. In some embodiments, the spread of an antibiotic resistance gene is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard. In some embodiments, the spread of a drug-resistance gene (e.g., an antibiotic resistance gene or MDR gene) comprises the spread from a first subject (e.g., a first subject) to a second subject (e.g., a second subject). In some embodiments, the oligosaccharide composition is administered in an effective amount and/or to a sufficient number of subject(s), that the rate at which a drug- or antibiotic-resistance gene, or an MDR element, is transferred from a first pathogen to a second pathogen is reduced. This transfer might be measured by showing the presence of a similar gene or toxin, identified by any of the methods described herein, present in a second pathogen distinct from the first pathogen. This distinction can be at the level of organism identification (e.g., metabolite production, species identity, or susceptibility to antibiotics), or by molecular methods to show other differences, such as any of those described herein.

In some embodiments, provided is a method of reducing the rate at which a pathogen causes infection or colonization (e.g., in a subject) by administering a oligosaccharide composition to the subject, e.g., in an effective amount and/or to a sufficient number of subjects that the rate of infection is reduced. In some embodiments, the oligosaccharide composition is administered in an effective amount and/or to a sufficient number of subject(s), that the rate at which a pathogen causes infection, or the severity of pathogen infection, as indicated by assessment of symptoms associated with infection, is reduced. In some embodiments, the rate of infection is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard.

In some embodiments, oligosaccharide compositions provided herein effectively prevent onset of a pathogenic infection (e.g., in immunocompromised (e.g., HSCT) patients or ICU patients). For example, oligosaccharide compositions may be provided prior to, during, or after a certain medical procedure or medical event to prevent onset of a pathogenic infection. In some embodiments, oligosaccharide compositions provided herein effectively prevent onset of a pathogenic infection in at least 1 out of every 100 subjects, at least 5 out of every 100 subjects, at least 10 out of every 100 subjects, at least 20 out of every 100 subjects, at least 30 out of every 100 subjects, at least 40 out of every 100 subjects, at least 50 out of every 100 subjects, at least 60 out of every 100 subjects, at least 70 out of every 100 subjects, at least 80 out of every 100 subjects, at least 90 out of every 100 subjects, or at least 95 out of every 100 subjects. In some embodiments, oligosaccharide compositions provided herein effectively prevent onset of a pathogenic infection in 10-100%, 10-20%, 15-25%, 20-50%, 40-60%, 50-75%, 60-80%, 75-90%, 80-100%, or 90-100% of subjects.

In some embodiments, oligosaccharide compositions provided herein effectively minimizes or prevents progression of an infection (e.g., progression from mild or moderate symptoms to severe symptoms). For example, oligosaccharide compositions may be provided prior to, during, or after the onset of infection to prevent progression of the infection. In some embodiments, oligosaccharide compositions provided herein effectively minimizes or prevents progression of an infection in at least 1 out of every 100 subjects, at least 5 out of every 100 subjects, at least 10 out of every 100 subjects, at least 20 out of every 100 subjects, at least 30 out of every 100 subjects, at least 40 out of every 100 subjects, at least 50 out of every 100 subjects, at least 60 out of every 100 subjects, at least 70 out of every 100 subjects, at least 80 out of every 100 subjects, at least 90 out of every 100 subjects, or at least 95 out of every 100 subjects. In some embodiments, oligosaccharide compositions provided herein effectively minimizes or prevents progression of an infection in 10-100%, 10-20%, 15-25%, 20-50%, 40-60%, 50-75%, 60-80%, 75-90%, 80-100%, or 90-100% of subjects.

Reduction in the rate of infection or colonization using a method described herein may be prospective or retrospective, e.g., relative to an infection. In some embodiments, the method described herein comprises monitoring a subject or a population of subjects for a similar infection, e.g., through observation of similar symptoms or similar features to those known to be caused by or identified with a pathogen of interest. Rather than, or in addition to using clinical characteristics, any of the methods described herein might be used to more specifically determine the type of the pathogen involved, and its relationship -if any- to spread or a reservoir.

In some embodiments, provided is a method of modulating the gastrointestinal tract (e.g., all of the GI tract or a part thereof, e.g., the small intestine, the large intestine, the colon, and the like) of a subject . In some embodiments, the method comprises modulating the environment (e.g., chemical or physical environment) of the gastrointestinal tract of a subject to make the gastrointestinal tract (and the microbial community therein) less selective or less receptive for a pathogen or an antibiotic resistance gene carrier. In some embodiments, the method further comprises administering a second agent in combination with a oligosaccharide composition, e.g., charcoal or an antibiotic-degrading enzyme (e.g., beta-lactamase), or a synbiotic (e.g., an engineered beta-lactamase (e.g., a non-infectious beta-lactamase).

In some embodiments, provided is a method of reducing the transfer of a drug-resistance gene (e.g., an antibiotic resistance gene or an MDR gene) from one organism (e.g., bacterial taxa, e.g., taxa containing the antibiotic resistance gene or an MDR gene) to another organism (e.g., taxa that do not contain the antibiotic resistance gene or an MDR gene) by administering a oligosaccharide composition to a subject , e.g., in an effective amount and/or to a sufficient number of subjects that the transfer of the drug-resistance gene (e.g., an antibiotic resistance gene or MDR gene) is reduced. In some embodiments, the method comprises reducing the transfer of a drug-resistance gene (e.g., an antibiotic resistance gene or an MDR gene) to an organism with increased pathogenic potential. In some embodiments, the method comprises reducing the number of recipient bacteria, (e.g., commensal bacterial strains), capable of taking up an antibiotic resistance gene, in a subject . In some embodiments, the method comprises reducing the probability of a pathogen or an antibiotic resistance gene carrier to spread or transfer an antibiotic resistance gene. In some embodiments, the method comprises reducing the ability of a pathogen or an antibiotic resistance gene carrier to reach a state of competency. Competency refers to the bacteria's ability to take up genes (e.g., antibiotic resistance genes) from the environment (von Wintersdorff et al. Front. Microbiol. (2016); 7: 173). In some embodiments, the method comprises reducing exchange of gene material (e.g., conjugation-based) in a pathogen or an antibiotic resistance gene carrier. In some embodiments, the method comprises reducing the level of free nucleic acid (e.g. microbial DNA, e.g., comprising an antibiotic resistance gene cassette), in a pathogen or antibiotic resistance gene carrier, e.g., after the pathogen or antibiotic resistance gene carrier reaches competency. In some embodiments, the method comprises increasing microbial metabolism of a nucleic acid (e.g. microbial DNA, e.g., comprising an antibiotic resistance gene cassette). In some embodiments, the method comprises use of a nucleic acid binding molecule as a scavenger, e.g., for binding to a pathogen-derived or antibiotic resistance gene carrier-derived nucleic acid.

In some embodiments, provided is a method of reducing pathogen infectivity, as determined by the incidence of the number of pathogens in a population. In some embodiments, the oligosaccharide composition is administered in an effective amount to reduce the number of pathogen cells that can transmit a drug or antibiotic resistance gene, or MDR element, to another organism. In some embodiments, the oligosaccharide composition is administered in an effective amount to reduce the number of organisms (e.g. bacteria) that can receive a drug or antibiotic resistance gene, or MDR element. In some embodiments, the oligosaccharide composition is administered in an effective amount to reduce the ability of a pathogen cell to enter the state in which it can donate a drug or antibiotic resistance gene, or MDR element, to another organism. In some embodiments, the oligosaccharide composition is administered in an effective amount to reduce the ability of a pathogen cell to enter the state in which it can receive a drug or antibiotic resistance gene, or MDR element, from another organism.

In some embodiments, provided is a method of reducing the presence of a drug or antibiotic resistance gene, or MDR element in a microbe (e.g., a pathogen, e.g. a bacterial pathogen) or microbial population. In some embodiments, the oligosaccharide composition is administered in an effective amount to reduce the copy number of a drug or antibiotic resistance gene, or MDR element, in a microbe (e.g. a bacterial pathogen, on a cell-by-cell basis) or reduce the total number in a microbial population. In some embodiments, the oligosaccharide composition is administered in an effective amount to increase the population of a gut microbe that is not a (potential) host for a drug or antibiotic resistance gene, or MDR element. In some embodiments, the oligosaccharide composition is administered in an effective amount to reduces the competence of a pathogen, e.g., Streptococcus, to take up a drug or antibiotic resistance gene, or MDR element. In some embodiments, the oligosaccharide composition is administered in an effective amount to reduces the ability of a bacterial cell (e.g., a pathogen), e.g., a gram-negative organism, e.g., E. coli or Klebsiella, to take up a drug or antibiotic resistance gene, or MDR element.

In some embodiments, the oligosaccharide composition is administered in an effective amount to shift the microbial community of a subject to displace or inhibit a pathogen, an organism that can donate a drug or antibiotic resistance gene, or MDR element (donor microbes), or an organism that can receive a drug or antibiotic resistance gene, or MDR element (recipient microbes). In some embodiments, the oligosaccharide composition is administered in an effective amount to reduce the probability of donor microbes to spread a drug or antibiotic resistance gene, or MDR element.

In some embodiments, provided is a method of managing an infection by a pathogen. In some embodiments, managing an infection by a pathogen comprises treating, preventing, and/or reducing the risk of developing an infection by a pathogen. In some embodiments, treating an infection by a pathogen comprises administering a oligosaccharide composition to a subject or population upon detection of a pathogen. In some embodiments, preventing an infection by a pathogen comprises administering a oligosaccharide composition to a subject or population at risk of developing an infection. The subject or population may include those who may have been exposed to the pathogen directly and/or infected individuals. In some embodiments, reducing the risk of developing an infection by a pathogen comprises administering a oligosaccharide composition to a subject or population that may become exposed to a pathogen.

In some embodiments, provided is a method to reduce the expression or release (e.g., by a pathogen) of a factor having an adverse effect on a subject such as a virulence factor or toxin. In some embodiments, the factor causes a disease. In some embodiments, a oligosaccharide composition is administered in an effective amount and/or to a sufficient number of subject(s), that the expression or release by a microbe (e.g., a pathogen) of a factor having an adverse effect on a subject, e.g., a virulence factor or a toxin, e.g., that causes disease, is reduced. In some embodiments, the expression of a factor (e.g., a virulence factor) is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard. An adverse effect in a subject , by such a factor, includes causing a disease, delaying diagnosis of a disease, or reducing the effectiveness of a disease treatment.

In some embodiments, provided is a method of modulating the number of gene donors (donor microbes) in a population (e.g., the state of competency/conjugation). In some embodiments, provided is a method of modulating the number of gene recipients (recipient microbes) in a population. In some embodiments, a oligosaccharide composition is administered in an effective amount to reduce the number of donor microbes (e.g., microbes that carry drug- or antibiotic-resistance genes or MDR elements). In some embodiments, a oligosaccharide composition is administered in an effective amount to reduce the number of recipient microbes. For example, a oligosaccharide composition may affect bacterial activity such that it reduces the frequency of transfer of toxins or determinants of antibiotic resistance between strains or species. This may be accomplished, for instance by transformation, conjugation, phage production or transduction, plasmid release or plasmid replication, such that fewer pathogens are able to access these toxins or resistance determinants. This in turn may reduce the availability of those markers to new pathogens. Such a reduction might be accomplished by modulating a state of competency or conjugation property.

In some embodiments, provided is a method of modulating the copy number of a resistance gene in a population. In some embodiments, the oligosaccharide composition is administered in an effective amount that the copy number of a drug- or antibiotic-resistance gene, toxin or virulence factor is reduced. For example, when fewer copies of the same genetic element are present there is generally a decline in its expression. Thus, a oligosaccharide composition resulting in decreased copy number of a drug or antibiotic resistance gene, toxin or virulence factor, might be expected to increase susceptibility to an antibiotic, reduce adverse effects of a pathogen, or reduce the availability of the gene, toxin or virulence factor to other microbial recipients. This might occur, for instance, due to reduced activity of or expression of addiction modules or other elements of importance for maintaining the copy number of the gene, toxin or resistance marker.

In some embodiments, provided is a method of modulating the fitness deficit (e.g., increase the burden of carrying a drug- or antibiotic-resistance gene or MDR element) of a population. In some embodiments, the modulating comprises increasing the burden of carrying a resistance gene. In some embodiments, the oligosaccharide composition is administered in an effective amount that the fitness deficit is increased. A oligosaccharide composition that increases the fitness deficit (e.g., caused by carrying or expressing a toxin, virulence factor or antibiotic resistance determinant) reduces the number of microbes (e.g., bacterial pathogens) carrying it, or their ability to persist in particular subjects (e.g., subjects). In some embodiments, the oligosaccharide composition alters the ecology of the GI tract (or a subset thereof, e.g., small intestine, large intestine, or colon) such that nitrogen sources is in short supply. This in turn can increase the cost of maintaining additional genetic elements by nucleic acid synthesis. In some embodiments, the fitness deficit results from enhanced recognition or response by the host (e.g., a human subject). For example, some factors, such as bacterial lipopolysaccharide (LPS) are directly recognized by human cells, resulting in immune responses.

Some pathogens (e.g., viruses and bacteria, e.g., Vibrio cholerae and Norovirus) have been shown to have glycan receptors, or glycan moieties that are necessary to infect gut cells (Holmer, et al. FEBS Letters 584 (2010) 2548-2555). In some embodiments, provided is a method of decreasing the binding of a pathogen to a cell, decreasing the activity of a pathogen on or in a cell, decreasing the entry of a pathogen into or onto a cell, or decreasing the effect of a pathogen on a cell, wherein the method comprises administering of a oligosaccharide composition in an effective amount and/or to a sufficient number of subjects to decrease the binding of a pathogen, its activity, entry into, or effect on a cell. In some embodiments, the cell is a human cell. In some embodiments, the oligosaccharide composition binding to a pathogen prevents said pathogen or another pathogen from reaching and entering a cell. In other embodiments, without being bound by theory, a oligosaccharide composition may directly or indirectly induce a modification of the gut lining or the mucous membrane, or affect another property such that pathogen entry into a cell, pathogen effect on a cell, the ability of a pathogen to persist within a cell or avoid antibody or immune recognition.

In some embodiments, provided is a method of modulating the anti-microbial output (e.g., immune response) of a subject. For example, a oligosaccharide composition is administered to increase mucus production, or antibody production or secretion, or the production of antimicrobial peptides (e.g., such as RegIIIγ) thereby increasing resistance to pathogens. RegIIIγ is an antimicrobial protein that binds intestinal bacteria via interactions with peptidoglycan carbohydrate (Cash et al., Science. (2006) Aug. 25; 313(5790): 1126-1130). The modulation of the immune response of the subject, e.g., in response to administering the oligosaccharide compositions described herein, may be determined by measuring one or more markers of inflammation, e.g. IFN-γ, IL-1β, IL-2, IL-4, IL-6, IL-8, IL-10, IL-12p70, IL-13, and TNF-α. The markers can be determined, e.g., from stool or blood samples.

In some embodiments, provided is a method of reducing a ratio of pathogenic bacteria to commensal bacteria (e.g., in the gastrointestinal tract of a subject). In some embodiments, a ratio of pathogenic bacteria to commensal bacteria is reduced by decreasing the abundance of pathogenic bacteria. In some embodiments, a ratio of pathogenic bacteria to commensal bacteria is reduced by increasing the abundance of commensal bacteria.

In some embodiments, provided is a method of modulating the microbial community composition and/or the metabolic output of the microbial community, e.g. modulating the environment, e.g., to modulate (e.g., reduce) pathogen growth. In some embodiments, a oligosaccharide composition is administered in an effective amount to modulate the microbial community and alter the environment of the GI tract, (e.g., altering pH, altering lactic acid, altering microbial density, etc.). In some embodiments, the method comprises outcompeting a pathogen, pathobiont or an antibiotic resistance gene carrier for space or nutrients in the gastrointestinal tract. In some embodiments, a oligosaccharide composition is administered in an effective amount to reduce the “space” for a pathogen or pathobiont to colonize, e.g., physical space. In some embodiments, the method comprises making non-pathogenic bacteria fitter (e.g., providing a more selective food source or encouraging growth of fitter (e.g., faster) growing species/strains). In some embodiments, the method comprises outcompeting a pathogen, pathobiont or an antibiotic resistance gene carrier by increasing the population of a commensal bacterial strain, or by increasing an anti-microbial defense mechanism in a commensal bacterial strain, e.g., production of a bacteriocin, anti-microbial peptide, hydrogen peroxide, or low pH (e.g., through increased level of an acid (e.g., acetate, butyrate, and the like).

In some embodiments, a ratio of pathogenic bacteria to commensal bacteria (e.g., in the gastrointestinal tract of a subject) is determined by performing nucleic acid sequencing (e.g., 16S metagenomic sequencing) of a fecal sample (e.g., collected from a subject prior to, or following treatment with the oligosaccharide composition). 16S metagenomic sequencing of a fecal sample may be accomplished, for example, by extracting genomic DNA from the fecal sample and performing standard 16S sequencing. In some embodiments, the variable region 4 of the 16S rRNA gene is amplified and sequenced (e.g., in accordance with the Earth Microbiome Project protocol www.earthmicrobiome.org/emp-standard-protocols/16s/ and/or Caporaso J G et al. Ultra-high-throughput microbial community analysis on the Illumina HiSeq and MiSeq platforms. ISME J. (2012) August; 6(8):1621-4)). Raw sequences may be demultiplexed, and each sample may be processed separately with UNOISE2 (Robert Edgar UNOISE2: improved error-correction for Illumina 16S and ITS amplicon sequencing. bioRxiv (2016) Oct. 15). Reads from 16S rRNA amplicon sequencing data may be rarefied to 5000 reads, without replacement, and the resulting OTU table used in downstream calculations. The analyzed sequencing data may allow for calculations of the total abundance of various bacterial species (e.g., pathogenic and commensal bacterial species), from which the relative abundance or absolute abundance of pathogenic and commensal bacteria may be determined.

In some embodiments, the reduction of a ratio of pathogenic bacteria to commensal bacteria (e.g., in the gastrointestinal tract of a subject) is determined by (i) performing 16S metagenomic sequencing of a fecal sample collected from the subject prior to administration of the oligosaccharide composition; (ii) performing 16S metagenomic sequencing of a fecal sample collected from the subject following administration of the oligosaccharide composition; and (iii) comparing the relative or absolute abundance of pathogens determined using the sequencing data provided in (ii) relative to the relative or absolute abundance of pathogens determined using the sequencing data provided in (i).

In some embodiments, provided are methods of reducing the spread of pathogens. In some embodiments, pathogens include bacterial pathogens (e.g., Abiotrophia spp., (e.g., A. defective), Achromobacter spp., Acinetobacter spp., (e.g., A. baumanii), Actinobaculum spp., (e.g., A. schallii), Actinomyces spp., (e.g., A. israelii), Aerococcus spp., (e.g., A. urinae), Aeromonas spp., (e.g., A. hydrophila), Aggregatibacter spp., e.g. A. aphrophilus, Bacillus anthracis, Bacillus cereus group, Bordetella spp., Brucella spp., e.g. B. henselae, Burkholderia spp., e.g., B. cepaciae, Campylobacter spp., e.g., C. jejuni, Chlamydia spp., Chlamydophila spp., Citrobacter spp., e.g., C. freundii, Clostridium botulinum, Clostridium difficile, Clostridium perfringens, Corynebacterium spp., e.g., C. amycolatum, Cronobacter, e.g., C. sakazakii, Enterobacteriaceae, including many of the genera below, Ehrlichia spp., Enterobacter spp., e.g., E. cloacae, Enterococcus spp., e.g. E. faecium, Escherichia spp., including enteropathogenic, uropathogenic, and enterohemorrhagic strains of E. coli, Francisella spp., e.g. F. tularensis, Fusobacterium spp., e.g. F. necrophorum, Gemella spp., e.g. G. mobillorum, Granulicatella spp., e.g. G. adiaciens, Haemophilus spp., e.g. H. influenza, Helicobacter spp., e.g. H. pylori, Kingella spp., e.g. K. kingae, Klebsiella spp., e.g. K. pneumoniae, Legionella spp., e.g. L. pneumophila, Leptospira spp., Listeria spp., e.g. L. monocytogenes, Morganella spp., e.g. M. morganii, Mycobacterium spp., e.g. M. abcessus, Neisseria spp., e.g. N. gonorrheae, Nocardia spp., e.g. N. asteroids, Ochrobactrum spp., e.g. O. anthropic, Pantoea spp., e.g. P. agglomerans, Pasteurella spp., e.g. P. multocida, Pediococcus spp., Plesiomonas spp., e.g. P. shigelloides, Proteus spp., e.g. P. vulgaris, Providencia spp., e.g. P. stuartii, Pseudomonas spp., e.g. P. aeruginosa, Raoultella spp., e.g. R. ornithinolytica, Rothia spp., e.g. R. mucilaginosa, Salmonella spp., e.g. S. enterica, Serratia spp., e.g. S. marcesens, Shigella spp., e.g. S. flexneri, Staphylococcus aureus, Staphylococcus lugdunensis, Staphylococcus pseudintermedius, Staphylococcus saprophyticus, Stenotrophomonas spp., e.g. S. maltophilia, Streptococcus agalactiae, Streptococcus anginosus, Streptococcus constellatus, Streptococcus dysgalactiae, Streptococcus intermedius, Streptococcus milleri, Streptococcus pseudopneumoniae, Streptococcus pyogenes, Streptooccus pneumoniae, Treponema spp., Ureaplasma ureolyticum, Vibrio spp., e.g. V. cholerae, and Yersinia spp., (e.g., E enterocolitica)); viral pathogens (e.g., Adenovirus, Astrovirus, Cytomegalovirus, Enterovirus, Norovirus, Rotavirus, and Sapovirus); and gastrointestinal pathogens (e.g., Cyclospora spp., Cryptosporidium spp., Entamoeba histolytica, Giardia lamblia, and Microsporidia, (e.g., Encephalitozoon canaliculi)).

In some embodiments, the method comprises reducing the spread of antibiotic resistant organisms. Antibiotic resistant organisms include: Beta-lactamase producing Enterobacteriaceae (including extended spectrum beta lactamase and carbapenemase producers, possessing genes such as TIM, OXA, VIM, SHV, CTX-M, KPC. NDM or AmpC); Vancomycin-resistant Enterococcus (e.g., possessing genes such as VanA or VanB); Fluoroquinolone-resistant Enterobacteriaceae (e.g., with genes such as Qnr); Carbapenem-resistant and multidrug resistant Pseudomonas; Methicillin-resistant Staphylococcus aureus and Streptococcus pneumoniae (e.g., possessing the MecA gene); Multidrug resistant Acinetobacter (often containing beta lactamase); Trimethoprim resistant organisms (e.g., dihydrofolate reductase); Sulfamethoxazole resistant organisms (e.g., dihydropteroate synthetase); and Aminoglycoside resistant organisms (e.g., ribosomal methyltransferase).

In some embodiments, provided is a method to manage an infection by a pathogen comprising, administering to a first and/or second subject, a second treatment. In some embodiments, the second treatment comprises administering charcoal, or other adsorbing agent. In this embodiment, the adsorbing agent might serve to reduce the presence of antibiotic within the GI tract (e.g., small intestine, large intestine, colon), so as to reduce the selective pressure of maintaining a resistance determinant, thereby allowing its reservoir, level, spread or adverse effect to be reduced. Alternatively, the adsorbing agent might increase the beneficial effect of the oligosaccharide composition. In some embodiments, the second treatment comprises administering a nonabsorbable antibiotic such as a beta lactam, or a beta lactamase inhibitor to a subject. In some embodiments, the second treatment comprises administering an antibiotic-degrading enzyme, e.g., beta-lactamase enzyme.

In some embodiments, the subject is critically ill and/or a transplant patient. Critically ill subjects and/or transplant patients are prone to infections (e.g. have a high rate of infections), such as bloodstream infections. In some embodiments, infectious microbes are carried in the gut (e.g., can be acquired through colonization) and include E. coli, Klebsiella, other Enterobacteriaceae, and Enterococcus. In some embodiments, the microbes (e.g., pathogens) are drug resistant (e.g. carbapenem-resistant Enterobacteriaceae, vancomycin-resistant Enterococcus).

In some embodiments, assessment of colonization (e.g., with pathogens) is used to predict the risk of infection (e.g., bloodstream infection, urinary tract infection (UTI), or respiratory infection, bacteremia), e.g., by correlating levels of colonization (e.g., by assessing a suitable sample for presence or absence of predetermined bacterial taxa and/or assessing pathogen load) with risk of infection, wherein evidence of colonization is correlated with an increased risk of infection, wherein culture-negative subjects are at lower risk of infection. In some embodiments, higher levels of bacteria lead to higher rates of infection. In some embodiments, intestinal colonization (e.g. by a pathogen, e.g. VRE) precedes infection in other tissues (e.g., bloodstream). Examples of gastrointestinal tract-colonizing pathogens may include: Enterobacteriaceae (e.g. E. coli, Klebsiella, Enterobacter, Proteus) and Enterococcus. In some embodiments, gastrointestinal tract-colonizing pathogens further include multidrug resistant bacteria (e.g., Carbapenem resistant Enterobacteriaceae, Vancomycin resistant Enterococcus).

In some embodiments, the outcome of screening subject populations for pathogen status determines the course of bloodstream infection management. In some embodiments, screening methods comprise stool sampling (e.g. by rectal swab) of subjects. In some embodiments, the method comprises assessing the presence/absence (abundance) of drug/antibiotic resistant pathogens (e.g., VRE) in the stool. In some embodiments, the level of pathogens within the gut is correlated with infection risk. In some embodiments, intensive care unit (ICU) subjects, transplant subjects, chemotherapy-receiving subjects, and antibiotic-receiving subjects have a higher risk of having pathogen colonization from antibiotic resistant bacteria such as carbapenem resistant Enterobacteriaciae and Vancomycin-resistant Enterococcus. In some embodiments, reducing the level of pathogens within the gut reduces risk (e.g., by administering a oligosaccharide composition if desired in combination with an antibiotic). In some embodiments, if the drug resistant pathogen is absent, the subject is administered a oligosaccharide composition to prevent infection (e.g., bloodstream infection) or bacteremia. In some embodiments, if the drug resistant pathogen is present, the subject is administered a oligosaccharide composition to reduce infection (e.g., bloodstream infection) or bacteremia.

In some embodiments, provided is a method to reduce the colonization level or prevalence of antibiotic resistant pathogens carried in the GI tract of high-risk subjects (e.g. subjects). Exemplary antibiotic resistant pathogens include Carbapenem-resistant Enterobacteriaciae (e.g., extended spectrum beta lactamase (ESBL) producing Enterobacteriaciae (ESBLE)) and Vancomycin-resistant Enterococcus.

In some embodiments, provided is a method to reduce the rate of infections (e.g., from pathogens that colonize the GI tract) in critically ill or high-risk subjects (e.g. subjects). In some embodiments, the method comprises reducing the rate of urinary tract infections. In some embodiments, the method comprises reducing the rate of bloodstream infections. In some embodiments, the method comprises reducing the rate of respiratory tract infections.

In some embodiments, the method comprises managing infections in subjects. Examples of subject groups with infections (bacteremia) include: subjects with urinary infections (e.g., infected with Enterococcus, Enterobacteriaciae), subjects with bloodstream infections (e.g., infected with Enterococcus, Enterobacteriaciae), transplant subjects (e.g., bone marrow (e.g., undergoing hematopoietic stem cell transplantation), solid organ (e.g., liver)), intensive care patients (e.g., infected with Carbapenem resistant Enterobacteriaciae and ESBL producing pathogens), pre-transplant liver failure patients (e.g., infected with Vancomycin resistant Enterococcus), post-transplant liver failure patients (e.g., infected with Vancomycin resistant Enterococcus). Subjects undergoing chemotherapy experience high levels of enteric pathogen bacteremia, C. difficile infection (CDI)C, and chemotherapy-induced diarrhea compared to other subjects (e.g., the general hospital patient population). In some embodiments, antibiotic-treated subjects comprise higher pathogen loads, including antibiotic resistant pathogens. In some embodiments, subjects undergoing or about to undergo a transplant, subjects with cancer, subjects with liver disease (e.g., end-stage renal disease), or subjects with suppressed immune system (e.g., immunocompromised subjects) may have high risk of developing infections, e.g., gut-derived infections. In some embodiments, the method comprises prophylactic treatment, e.g., with a oligosaccharide composition, of a subject, e.g., a subject with a high risk of developing an infection. In some embodiments, subjects who are undergoing chemotherapy or antibiotic treatment have reduced diversity of commensal bacteria. In some embodiments, the method comprises treatment of a subject to reduce the colonization of pathogens, e.g., multidrug resistant pathogens, in a subject, e.g., subjects in a facility, e.g., a hospital or long-term care facility. In some embodiments, the method comprises treatment of a subject to reduce the transmission of pathogens, e.g., multidrug resistant pathogens, from a first subject to a second subject, e.g., subjects in a facility, e.g., a hospital or long-term care facility. In some embodiments, bacteria that pose a risk of colonization in subjects (or a capable of colonizing the GI tract of subjects) comprise resistant subpopulations of Enterobacteriaceae (e.g., E. cloacae), Enterococcus, C. difficile (including Nap1 (pandemic hypervirulent) C. difficile strain), and bacteria that cause infectious diarrhea (e.g., Campylobacter, Salmonella, Shigella, enterohemorrhagic E. coli (EHEC), enterotoxigenic E. coli (ETEC), enteropathogenic E. coli (EPEC), enteroinvasive E. coli (EIEC), enteroaggregative E. coli (EAEC), diffusely adherent E. coli (DAEC), and uropathogenic E. coli).

In some embodiments, the method comprises managing infections in subjects who are in need of an organ transplant, e.g., a liver or bone marrow transplant. In some embodiments, the method comprises managing infections in subjects immediately, or shortly, before said subject receives an organ transplant, e.g., a liver or bone marrow transplant. In some embodiments, the method comprises managing infections in subjects immediately, or shortly, after said subject receives an organ transplant, e.g., a liver or bone marrow transplant. In some embodiments, the method comprises managing infections in subjects who have, are suspected of having, or at risk of having end-stage liver disease (ESLD).

In some embodiments, the method reduces a ratio of pathogenic bacteria to commensal bacteria, e.g., in a subject, e.g., in the gastrointestinal tract of the subject (e.g, the colon). In some embodiments, a ratio of pathogenic bacteria to commensal bacteria is reduced by at least 5%, 10%, 20%, 30%, 40%, 50%, 60%, 70%, 80%, 90%, 100%, 125%, 150%, or 200%. In some embodiments, a ratio of pathogenic bacteria to commensal bacteria is reduced by 1-10%, 5-20%, 10-25%, 20-40%, 30-50%, 40-60%, 50-70%, 60-80%, 70-90%, 80-100%, 90-110%, 100-125%, 110-150%, 125-175%, or 150-200%.

In some embodiments, the method reduces the abundance of pathogens and/or increases the abundance of commensal bacteria, e.g., in a subject, e.g., in the gastrointestinal tract of the subject (e.g, the colon). In some embodiments, the method increases the alpha-diversity (e.g., a high degree of diversity) of a microbial community (e.g., a community of commensal bacteria), e.g., of the gut of a subject.

In some embodiments, the method reduces the abundance of pathogens by at least 5%, 10%, 20%, 30%, 40%, 50%, 60%, 70%, 80%, 90%, 100%, 125%, 150%, or 200%. In some embodiments, the method reduces the abundance of pathogens by 1-10%, 5-20%, 10-25%, 20-40%, 30-50%, 40-60%, 50-70%, 60-80%, 70-90%, 80-100%, 90-110%, 100-125%, 110-150%, 125-175%, or 150-200%.

In some embodiments, the method increases the abundance of commensal bacteria by at least 5%, 10%, 20%, 30%, 40%, 50%, 60%, 70%, 80%, 90%, 100%, 125%, 150%, or 200%. In some embodiments, the method increases the abundance of commensal bacteria by 1-10%, 5-20%, 10-25%, 20-40%, 30-50%, 40-60%, 50-70%, 60-80%, 70-90%, 80-100%, 90-110%, 100-125%, 110-150%, 125-175%, or 150-200%.

In some embodiments, the method increases the alpha-diversity (e.g., a high degree of diversity) of the microbiome in the gastrointenstinal tracts. Alpha diversity may be measured using the Shannon index in combination with nucleic acid sequencing. In some embodiments, the Shannon Index indicates that subjects in need of treatment (e.g., ICU patients) have communities that are considerably less diverse in their representation of bacterial taxa than healthy subjects. In some embodiments, community richness, or the number of unique taxa within a sample, is considerably lower in subjects in need of treatment (e.g., ICU patients) relative to healthy subjects.

In some embodiments, oligosaccharide compositions are substantially fermented or consumed by commensal bacteria and are not fermented or consumed by pathogens. In some embodiments, oligosaccharide compositions are substantially fermented or consumed by commensal bacteria and are fermented or consumed by pathogens at low levels. In some embodiments, a oligosaccharide composition that is substantially consumed by commensal bacteria may increase the diversity and biomass of the commensal microbiota and lead to a reduction in the relative abundance or absolute abundance of a pathogen(s), such as a bacterial pathogen (e.g., a pathogenic taxa). In some embodiments, a oligosaccharide composition is substantially non-fermented or not consumed by VRE or CRE species. In some embodiments, a oligosaccharide composition is substantially non-fermented or not consumed by C. difficile.

In some embodiments, a oligosaccharide composition supports the growth of commensal or probiotic bacteria, e.g., in a gut microbiome. In some embodiments, a oligosaccharide composition does not support the growth of at least one pathogen, e.g., does not support the growth of a CRE, VRE, and/or C. difficile species.

In some embodiments, administration of a oligosaccharide composition may increase the concentration, amount or abundance (e.g., relative abundance or absolute abundance) of commensal bacteria relative to pathogenic bacteria in the microbiome of a subject (e.g., a human patient). In some embodiments, administration of a oligosaccharide composition and a population of viable commensal or probiotic bacteria may increase the concentration, amount, or abundance (e.g., relative abundance or absolute abundance) of commensal bacteria relative to pathogenic bacteria in the microbiome of a subject (e.g., a human patient). In some embodiments, administration of a oligosaccharide composition that supports the growth of commensal or probiotic bacteria, e.g., in a gut microbiome, may increase the concentration, amount or abundance (e.g., relative abundance or absolute abundance) of commensal bacteria relative to pathogenic bacteria in the microbiome of a subject (e.g., a human patient). In some embodiments, administration of a oligosaccharide composition that does not support the growth of at least one pathogen, e.g., does not support the growth of a CRE, VRE, and/or C. difficile species, e.g., in a gut microbiome, may increase the concentration, amount or abundance (e.g., relative abundance or absolute abundance) of commensal bacteria relative to pathogenic bacteria in the microbiome of a subject (e.g., a human patient). In some embodiments, administration of a oligosaccharide composition that supports the growth of commensal or probiotic bacteria and does not support the growth of at least one pathogen, e.g., does not support the growth of a CRE, VRE, and/or C. difficile species, e.g., in a gut microbiome, may increase the concentration, amount or abundance (e.g., relative abundance or absolute abundance) of commensal bacteria relative to pathogenic bacteria in the microbiome of a subject (e.g., a human patient).

In some embodiments, administration of an oligosaccharide composition may increase the concentration, amount or abundance (e.g., relative abundance or absolute abundance) of Bacteroidetes (e.g., Bacteroidales) relative to pathogenic bacteria in the microbiome of a subject (e.g., a human patient).

In embodiments, an oligosaccharide composition described herein is co-administered with commensal or probiotic bacterial taxa and bacteria that are generally recognized as safe (GRAS) or known commensal or probiotic microbes. In some embodiments, probiotic or commensal bacterial taxa (or preparations thereof) may be administered to a subject before or after administration of an oligosaccharide composition to the subject. In some embodiments, probiotic or commensal bacterial taxa (or preparations thereof) may be administered to a subject simultaneously with administration of an oligosaccharide composition to the subject.

In embodiments, an oligosaccharide composition described herein is administered with a population of Bacteroidetes. In embodiments, an oligosaccharide composition described herein is administered with a population of Bacteroidales.

A commensal or probiotic bacteria is also referred to a probiotic. Probiotics can include the metabolites generated by the probiotic bacteria during fermentation. These metabolites may be released to the medium of fermentation, e.g., into a host organism (e.g., subject), or they may be stored within the bacteria. Probiotic bacteria includes bacteria, bacterial homogenates, bacterial proteins, bacterial extracts, bacterial ferment supernatants and combinations thereof, which perform beneficial functions to the host animal, e.g., when given at a therapeutic dose.

Useful probiotics include at least one lactic acid and/or acetic acid and/or propionic acid producing bacteria, e.g., microbes that produce lactic acid and/or acetic acid and/or propionic acid by decomposing carbohydrates such as glucose and lactose. Preferably, the probiotic bacteria is a lactic acid bacterium. In embodiments, lactic acid bacteria include Lactobacillus, Leuconostoc, Pediococcus, Streptococcus, and Bifidobacterium. Suitable probiotic bacteria can also include other bacterias which beneficially affect a host by improving the hosts intestinal microbial balance, such as, but not limited to yeasts such as Saccharomyces, Debaromyces, Candida, Pichia and Torulopsis, molds such as Aspergillus, Rhizopus, Mucor, and Penicillium and Torulopsis, and other bacteria such as but not limited to the genera Bacteriodes, Clostridium, Fusobacterium, Melissococcus, Propionibacterium, Enterococcus, Lactococcus, Staphylococcus, Peptostreptococcus, Bacillus, Pediococcus, Micrococcus, Leuconostoc, Weissella, Aerococcus, and Oenococcus, and combinations thereof.

Non-limiting examples of lactic acid bacteria useful in the disclosure herein include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus paracasei, Lactobacillus brevis, Bifidobacterium longum, Bifidobacterium infantis, Bifidobacterium bifidum, Bifidobcterium animalis, Bifidobcterium lactis, Bifidobcterium breve, Bifidobcterium adolescentis, and Pediococcus cerevisiae and combinations thereof, in particular Lactobacillus, Bifidobacterium, and combinations thereof.

Commensal or probiotic bacteria which are particularly useful in the present disclosure include those which (for human administration) are of human origin (or of the origin of the mammal to which the probiotic bacteria is being administered), are non-pathogenic to the host, resist technological processes (i.e. can remain viable and active during processing and in delivery vehicles), are resistant to gastric acidity and bile toxicity, adhere to gut epithelial tissue, have the ability to colonize the gastrointestinal tract, produce antimicrobial substances, modulate immune response in the host, and influence metabolic activity (e.g. cholesterol assimilation, lactase activity, vitamin production).

The commensal or probiotic bacteria can be used as a single strain or a combination of multiple strains, wherein the total number of bacteria in a dose of probiotic bacteria is from about 1×10³ to about 1×10¹⁴, or from about 1×10 to about 1×10¹², or from about 1×10⁷ to about 1×10¹¹ CFU per dose.

The commensal or probiotic bacteria can be formulated with the oligosaccharide compositions while the probiotic bacteria are alive but in a state of “suspended animation” or somnolence. Once freeze-dried, the viable cultures(s) of probiotic bacteria are handled so as to minimize exposure to moisture that would reanimate the cultures because, once reanimated, the cultures can experience high rates of morbidity unless soon cultured in a high moisture environment or medium. Additionally, the cultures are handled to reduce possible exposure to high temperatures (particularly in the presence of moisture) to reduce morbidity.

The probiotic bacterias can be used in a powdered, dry form. The probiotic bacterias can also be administered in the oligosaccharide composition or in a separate oligosaccharide composition, administered at the same time or different time as the oligosaccharide compositions.

Other probiotic bacteria suitable include Bifidobacterium lactis, B. animalis, B. bifidum, B. longum, B. adolescentis, and B. infantis.

In embodiments, a commensal bacterial taxa that can be used in and/or in combination with an oligosaccharide composition described herein comprises Akkermansia, Anaerococcus, Bacteroides, Bifidobacterium (including Bifidobacterium lactis, B. animalis, B. bifidum, B. longum, B. adolescentis, B. breve, and B. infantis), Blautia, Clostridium, Corynebacterium, Dialister, Eubacterium, Faecalibacterium, Finegoldia, Fusobacterium, Lactobacillus (including, L. acidophilus, L. helveticus, L. bifidus, L. lactis, L. fermentii, L. salivarius, L. paracasei, L. brevis, L. delbruekii, L. thermophiles, L. crispatus, L. casei, L. rhamnosus, L. reuteri, L. fermentum, L. plantarum, L. sporogenes, and L. bulgaricus), Peptococcus, Peptostreptococcus, Peptoniphilus, Prevotella, Roseburia, Ruminococcus, Staphylococcus, and/or Streptococcus (including S. lactis, S. cremoris, S. diacetylactis, S. thermophiles).

In embodiments, a commensal bacterial taxa, e.g., GRAS strain, that can be used in and/or in combination with an oligosaccharide composition described herein comprises Bacillus coagulans GBI-30, 6086; Bifidobacterium animalis subsp. Lactis BB-12; Bifidobacterium breve Yakult; Bifidobacterium infantis 35624; Bifidobacterium animalis subsp. Lactis UNO 19 (DR10); Bifidobacterium longum BB536; Escherichia coli M-17; Escherichia coli Nissle 1917; Lactobacillus acidophilus DDS-1; Lactobacillus acidophilus LA-5; Lactobacillus acidophilus NCFM; Lactobacillus casei DN 114-001 (Lactobacillus casei Immunitas(s)/Defensis); Lactobacillus casei CRL431; Lactobacillus casei F19; Lactobacillus paracasei Stl 1 (or NCC2461); Lactobacillus johnsonii Lai (Lactobacillus LCI, Lactobacillus johnsonii NCC533); Lactococcus lactis L1A; Lactobacillus plantarum 299V; Lactobacillus reuteri ATTC 55730 (Lactobacillus reuteri SD2112); Lactobacillus rhamnosus ATCC 53013; Lactobacillus rhamnosus LB21; Saccharomyces cerevisiae (boulardii) lyo; mixture of Lactobacillus rhamnosus GR-1 and Lactobacillus reuteri RC-14; mixture of Lactobacillus acidophilus NCFM and Bifidobacterium lactis BB-12 or BL-04; mixture of Lactobacillus acidophilus CL1285 and Lactobacillus casei; and a mixture of Lactobacillus helveticus R0052, Lactobacillus rhamnosus R0011, and/or Lactobacillus rhamnosus GG (LGG).

In some embodiments, the method comprises the administration of a oligosaccharide composition and the administration of a commensal or probiotic bacterial species. In some embodiments, the combined administration of oligosaccharide compositions and commensal bacteria may be used to benefit patients with depleted microbiomes (e.g., patients with few or no detectable commensal bacteria), e.g., patients who are undergoing chemotherapy or receiving antibiotics. In some embodiments, the combined administration of oligosaccharide compositions and commensal bacteria may be used to benefit a subject or patient having a gut microbiome devoid of any detectable commensal bacteria. In some embodiments, the method comprises combined administration of oligosaccharide compositions and commensal bacteria to a subject or patient who has a gut microbiome devoid of any detectable commensal bacteria.

In some embodiments, an oligosaccharide composition described herein can be used for treating an urea cycle disorder (UCD) in a human subject. In some embodiments, the subject has hepatic encephalopathy (HE). In some embodiments, an oligosaccharide composition described herein can be used for treating a subject having end-stage liver disease (ESLD). In some embodiments, the subject is of a pediatric population (e.g., 2-24 months or 2-18 years old).

In some embodiments, an oligosaccharide composition described herein can be used for treating an infection, e.g., a bacterial infection, e.g. associated with a bacterial pathogen or pathobiont. In some embodiments, an oligosaccharide composition described herein can be used for treating an infection, e.g., a bacterial infection, e.g. associated with a antibiotic-resistant (e.g., multi-drug-resistant) bacterial pathogen In some embodiments, an oligosaccharide composition described herein can be used for treating an infection, e.g., a fungal infection.

In some embodiments, an oligosaccharide composition described herein can be used for reducing the relative or absolute abundance of pathogens or pathobionts in the human subject (e.g., in the gastrointestinal tract, the UTI tract, the bloodstream, or a different site, e.g. within the cardiovascular system or the respiratory system).

To treat infections, e.g., by reducing the relative or absolute abundance of pathogens or pathobionts in the human subject, the oligosaccharide composition is administered in an amount effective to modulate (e.g. reduce or inhibit) colonization or to modulate (e.g. increase) decolonization by the pathogen, e.g., in the gut (e.g., small intestine, large intestine and/or colon) of the human subject. In some embodiments, treatment includes one or both of (i) reducing the abundance of pathogenic bacteria, e.g., in the gastrointestinal tract, relative to a control (e.g., a control subject or baseline measurement), and (ii) increasing the abundance of commensal bacteria, e.g., in the gastrointestinal tract, relative to a control (e.g., a control subject or baseline measurement). In some embodiments, a subject for the methods described herein (e.g., treatment of infection, or methods reducing the relative or absolute abundance of pathogens) and who benefits from administration of the oligosaccharide composition described herein is a human patient. In some embodiments, the methods relate to a subject who is a patient receiving broad spectrum antibiotics. In some embodiments, the methods relate to a subject who is particularly susceptible to pathogen infection, e.g., the subject is critically-ill and/or immunocompromised. In some embodiments, the methods relate to a subject who is a patient having a lower abundance of commensal bacteria relative to a healthy subject in their gastrointestinal tract (e.g., their colon or intestines).

In some embodiments, the methods relate to a subject who has received or is currently receiving cancer treatment. In some embodiments, the methods relate to a subject who has received or is currently receiving immunosuppression. In some embodiments, the methods relate to a subject who is preparing for or recovering from a gastrointestinal surgery. In some embodiments, the methods relate to a subject who is a patient in an intensive care unit (ICU). In some embodiments, the methods relate to a subject who is a healthy subject. In some embodiments, the methods relate to a subject who is asymptomatic, yet is detectably colonized by pathogens. In some embodiments, the methods relate to a subject who is at risk of developing a pathogenic infection (e.g., and treatment with the oligosaccharide composition reduces the likelihood of infection). In some embodiments, the methods relate to a subject who is a transplant recipient or is preparing to receive a transplant. In some embodiments, the methods relate to a subject who is a hematopoietic stem cell treatment (HSCT) recipient or is preparing to receive a hematopoietic stem cell treatment. In some embodiments, the methods relate to a subject who is a solid organ transplant recipient or is preparing to receive a solid organ transplant. A subject undergoing any treatment or surgery (e.g., cancer treatment, gastrointestinal surgery, transplantation surgery) may be treated with an oligosaccharide composition before, during, and/or after the treatment or surgery. In some embodiments, treatment with an oligosaccharide composition before, during, and/or after the other treatment or surgery (e.g., in an ICU facility) prevents a pathogenic infection. In some embodiments, treatment with an oligosaccharide composition before, during, and/or after the other treatment or surgery (e.g., in an ICU facility) prevents graft-vs-host disease (GvHD).

In some embodiments, the methods relate to a subject who has an auto-immune disease (e.g., systemic lupus erythematosus, rheumatoid arthritis, Sjögren's syndrome, or Crohn's disease). In some embodiments, the methods relate to a subject who has a hematological malignancy. In some embodiments, the methods relate to a subject who has cirrhosis. In some embodiments, the methods relate to a subject who has a positive stool culture for Carbapenem-resistant Enterobacteriaciae (CRE), extended spectrum beta lactamase (ESBL) producing Enterobacteriaciae (ESBLE), and/or Vancomycin-resistant Enterococcus (VRE). In some embodiments, the methods relate to a subject who has low diversity of bacterial communities in the gastrointestinal tract. In some embodiments, the methods relate to a subject who has or is at risk of developing end-stage liver disease (ESLD). In some embodiments, the methods relate to a subject who has had multiple courses of antibiotics, and/or chronic use of antibiotics and/or has been overprescribed antibiotics. In some embodiments, the methods relate to a subject who is experiencing or is at risk of an over-aggressive immune response such as, for example, a cytokine storm.

A cytokine storm (also known as hypercytokinemia), in some embodiments, involves an immune reaction in which the body releases too many cytokines into the blood too quickly (e.g., at the same time). The release of a large amount of cytokines at one time can be harmful. In some embodiments, a cytokine storm is characterized by high fever, inflammation (e.g., redness and swelling), severe fatigue and/or nausea. In some embodiments, a cytokine storm is severe or life threatening and/or can lead to multiple organ failure.

In some embodiments, the methods (e.g., methods of treatment) provided herein comprise the administration of an oligosaccharide composition concurrent with administration of antibiotics or any other standard-of-care. In some embodiments, the methods provided herein comprise the administration of an oligosaccharide composition subsequent to administration of antibiotics or any other standard-of-care. In some embodiments, an oligosaccharide composition provides an additive benefit to subjects being administered antibiotics or any other standard-of-care. In some embodiments, an oligosaccharide composition provides an synergistic benefit (e.g., by reducing the ratio of pathogenic bacteria to commensal bacteria) to subjects being administered antibiotics or any other standard-of-care. In some embodiments, the antibiotics are broad spectrum antibiotics. In some embodiments, the antibiotics are intravenous Gram positive (e.g., vancomycin) or Gram-negative (e.g., ceftriaxone, cefepime, or piperacillin-tazobactam) antibiotics, or both Gram positive and Gram-negative agents together.

### IV. Kits

Kits also are contemplated. For example, a kit can comprise unit dosage forms of the oligosaccharide composition, and a package insert containing instructions for use of the composition in treatment. In some embodiments, the composition is provided in a dry powder format. In some embodiments, the composition is provided in solution. The kits include an oligosaccharide composition in suitable packaging for use by a subject in need thereof. Any of the compositions described herein can be packaged in the form of a kit. A kit can contain an amount of an oligosaccharide composition sufficient for an entire course of treatment, or for a portion of a course of treatment. Doses of an oligosaccharide composition can be individually packaged, or the oligosaccharide composition can be provided in bulk, or combinations thereof. Thus, in one embodiment, a kit provides, in suitable packaging, individual doses of an oligosaccharide composition that correspond to dosing points in a treatment regimen, wherein the doses are packaged in one or more packets.

Kits can further include written materials, such as instructions, expected results, testimonials, explanations, warnings, clinical data, information for health professionals, and the like. In one embodiment, the kits contain a label or other information indicating that the kit is only for use under the direction of a health professional. The container can further include scoops, syringes, bottles, cups, applicators or other measuring or serving devices.

### EXAMPLES

### Example 1: Reduction of Pathogen Abundance in a Defined Microbial Community in the Presence of Oligosaccharide Compositions

Approximately four hundred and fifteen different oligosaccharide compositions were tested for their ability to modulate (e.g., reduce) the abundance (e.g., relative abundance or absolute abundance) of pathogens and to support the growth of commensal bacteria in a defined microbial community (comprising 46 different commensal bacterial strains). This screening was conducted by spiking the defined microbial community with three drug-resistant bacteria (CRE Klebsiella pneumoniae, CRE Escherichia coli, and VRE Enterococcus faceium) and subsequently growing the spiked microbial community in the presence of a single test oligosaccharide composition, wherein the single test oligosaccharide composition represented the sole carbon source.

The defined microbial community was constructed by combining 46 strains that belonged to phyla Actinobacteria, Firmicutes, and Bacteroidetes: Blautia producta, Blautia hansenii, Clostridium celatum, Bacteroiodes cellulosilyticus, Odoribacter splanchnicus, Bifidobacterium catenulatum, Eubacterium hallii, Bacteroides dorei, Bifidobacterium pseudocatenulatum, Bifidobacterium adolescentis , Bacteroides coprophilus, Lactobacillus casei, Coprococcus catus, Bifidobacterium angulatum, Eubacterium ventriosum, Lachnospira multipara, Parabacteroides merdae, Bacteroides finegoldii, Parabacteroides distasonis, Bacteroides thetaiotaomicron, Blautia hydrogenotrophica, Blautia coccoides, Clostridium bolteae, Clostridium scindens, Holdemanella biformis, Bifidobacterium longum sub. Infantalis, Ruminococcus obeum, Dorea formicigenerans, Collinsella aerofaciens, Eubacterium eligens, Faecalibacterium prausnitzii, Bifidobacterium longum, Prevotella copri, Eubacterium rectale, Bacteroides uniformis, Succinivibrio dextrinosolvens, Roseburia intestinalis, Clostridium nexile, Bacteroides caccae, Bacteroides vulgatus, Dorea longicatena, Akkermansia muciniphila, Bacteroides thetaiotaomicron, Bacteroides cellulosilyticus, Clostridium symbiosum, and Ruminococcus gnavus.

To prepare the defined microbial community, each of the 46 different commensal bacterial strains were independently grown in standard chopped meat glucose medium (CMG) for 18-48 hours, depending on the strain. After growth, the optical density (OD₆₀₀) of each bacterial strain was adjusted to 0.2 and equal volumes of each of the 46 strains were combined into one bottle at a final glycerol concentration of 15%. 1.5-mL aliquots of the defined microbial community were frozen at −80°. The CRE Klebsiella pneumoniae, CRE Escherichia coli, and VRE Enterococcus faecium strains for use in this Example were obtained from the Centers for Disease Control (CDC) and were grown aerobically in BHI medium for 12 hours at 37° C. prior to their addition to the defined microbial community.

Frozen aliquots of defined microbial community sample were later thawed and washed with a medium consisting of 900 mg/L sodium chloride, 26 mg/L calcium chloride dihydrate, 20 mg/L magnesium chloride hexahydrate, 10 mg/L manganese chloride tetrahydrate, 40 mg/L ammonium sulfate, 4 mg/L iron sulfate heptahydrate, 1 mg/L cobalt chloride hexahydrate, 300 mg/L potassium phosphate dibasic, 1.5 g/L sodium phosphate dibasic, 5 g/L sodium bicarbonate, 0.125 mg/L biotin, 1 mg/L pyridoxine, 1 mg/L pantothenate, 75 mg/L histidine, 75 mg/L glycine, 75 mg/L tryptophan, 150 mg/L arginine, 150 mg/L methionine, 150 mg/L threonine, 225 mg/L valine, 225 mg/L isoleucine, 300 mg/L leucine, 400 mg/L cysteine, and 450 mg/L proline (Theriot C M et al. Nat Commun. 2014; 5:3114) that was further supplemented with 0.1% peptone and 0.75 mM urea in order to adjust the defined microbial community sample to an OD₆₀₀ of 0.01. CRE Klebsiella pneumoniae, CRE Escherichia coli, and VRE Enterococcus faecium, all at OD₆₀₀ of 0.01, were then added to (i.e., used to spike) the defined microbial community sample. Each spiked aliquot was provided one of the four hundred and fifteen different oligosaccharide compositions (as the sole carbon source present in the aliquot) at a final concentration of 0.5% w/v or 0.05% w/v and incubated for 24 hours in the anaerobic chamber at 37° C. Water was used as a negative control (i.e., no carbon source). Each oligosaccharide composition was replicated up to 3 times. After 24 hours of incubation, the OD₆₀₀ of each spiked microbial community was measured to provide an approximation of total anaerobic growth.

To determine the level of pathogens in each spiked microbial community, a 200× dilution of each community was made in fresh Luria-Bertani (LB) media, and incubated aerobically for 24 hours at 37° C. OD₆₀₀ was measured every 15 minutes to generate growth curves using a Biotek plate reader. The time to mid-log growth was calculated and used to determine the total pathogen load at the end of the anaerobic phase of the experiment. Lesser times to mid-log growth corresponded to higher pathogen levels.

To identify oligosaccharide compositions that supported overall community growth, while reducing pathogen growth, both OD₆₀₀ at the end of the anaerobic phase (higher is a sign of more commensals) and time to mid-log growth during the secondary aerobic growth were normalized within their respective metrics from 0 to 1. These two values were multiplied and subtracted from 1 (i.e., 1−(anerobic growth*aerobic growth)). These final values, representative of individual oligosaccharide compositions, were normalized to negative control (water) to identify oligosaccharide compositions that reduced levels of pathogenic bacteria and promoted levels of commensal bacteria.

### Example 2. Reduction of Pathogen Abundance in a Fecal Suspensions from Humans in the Presence of Oligosaccharide Compositions

One hundred and thirty-five oligosaccharide compositions that reduced the abundance of pathogens and supported commensal growth in the spiked microbial community of Example 1 were further assessed for their abilities to similarly function in ex vivo fecal suspensions from humans that were spiked with single pathogen strains (VRE E. faecium, CRE K. pneumoniae, or CRE E. coli). Oligosaccharide compositions were prepared at 5% w/v in water, filter-sterilized and added to 96-well deep well microplates assay plates for a final concentration of 0.5% or 0.05% w/v in the assay, with water supplied as a negative control.

A human fecal sample donation was stored at −80° C. To prepare working stocks of fecal suspension, the fecal sample was transferred into the anaerobic chamber and allowed to thaw. The fecal sample was then prepared in 20% w/v in phosphate buffered saline (PBS) pH 7.4 (P0261, Teknova Inc., Hollister, Calif.), 15% glycerol. The 20% w/v fecal suspension +15% glycerol was centrifuged at 2,000×g, supernatant was removed, and the pellet was suspended in 1% PBS prior to dilution in a CM medium consisting of 900 mg/L sodium chloride, 26 mg/L calcium chloride dihydrate, 20 mg/L magnesium chloride hexahydrate, 10 mg/L manganese chloride tetrahydrate, 40 mg/L ammonium sulfate, 4 mg/L iron sulfate heptahydrate, 1 mg/L cobalt chloride hexahydrate, 300 mg/L potassium phosphate dibasic, 1.5 g/L sodium phosphate dibasic, 5 g/L sodium bicarbonate, 0.125 mg/L biotin, 1 mg/L pyridoxine, 1 m/L pantothenate, 75 mg/L histidine, 75 mg/L glycine, 75 mg/L tryptophan, 150 mg/L arginine, 150 mg/L methionine, 150 mg/L threonine, 225 mg/L valine, 225 mg/L isoleucine, 300 mg/L leucine, 400 mg/L cysteine, and 450 mg/L proline (Theriot C M et al. Nat Commun. 2014; 5:3114) that was further supplemented with 750 μM urea to provide a final dilution of 1% w/v fecal suspension.

One day prior to the start of the experiment, a single strain of CRE K. pneumoniae, a single strain of CRE E. coli, and a single strain of VRE were independently grown overnight in CM medium with 0.5% D-glucose in an anaerobic chamber. On the day of the experiment, aliquots of the pathogenic cultures were washed with PBS and the optical density (0D600) of each pathogenic culture and the 1% fecal suspension were adjusted to OD 0.1 in CM media. Each of the three pathogen cultures was then separately added to three aliquots of the fecal suspension such that the pathogen cultures comprised 8% of the final volume of the fecal suspension/pathogen mixture. Each of the three fecal suspension/pathogen mixtures were exposed to the 96-well plates of oligosaccharide compositions at a final concentration of 0.05% w/v or 0.5% w/v, 350 μL final volume per well, at 37° C. for 45 hours, anaerobically.

Following the ex vivo incubation, the plates were removed from the anaerobic chamber and a 200× dilution of each culture in fresh Luria-Bertani (LB) medium was made. These diluted cultures were incubated aerobically for 24 hours at 37° C. OD₆₀₀ was measured every 15 minutes for 24 hours to generate growth curves using a Biotek plate reader. The time to mid-log growth was calculated and used to determine the total pathogen load at the end of the anaerobic phase of the experiment. Lower time to mid-log values corresponded to higher levels of pathogens at the end of the anaerobic phase of the experiment.

Commensal strains in the fecal suspension communities are strict anaerobes and thus do not grow under aerobic conditions. The OD₆₀₀ measured at the end of anaerobic incubation (referred to as anaerobic OD₆₀₀) and time to mid-log growth as calculated from aerobic growth curves allowed for the identification of a selected oligosaccharide composition that exclusively supports commensal growth (e.g., high anaerobic OD₆₀₀ and long time to midlog).

### Example 3: Testing of Ability Oligosaccharide Compositions to Support the Growth of Single Pathogens

A total of fifty-five oligosaccharide compositions from Example 2 were further selected for investigation in an additional assay designed to directly test whether the oligosaccharide compositions can support the growth of single pathogens.

Individual pathogenic bacterial strains, including CRE Escherichia coli, CRE Klebsiella pneumoniae, and Clostridium difficile, were grown in CM, and single strain of VRE Enterococcus faecium was grown in mega medium (MM) prior to the addition of a single glycan preparation or water (a no carbon control). Mega Medium (MM) contains 10 g/L tryptone peptone, 5 g/L yeast extract, 4.1 mM L-cysteine, 100 mM potassium phosphate buffer (pH 7.2), 0.008 mM magnesium sulfate, 4.8 mM sodium bicarbonate, 1.37 mM sodium chloride, 5.8 mM vitamin K, 0.8% calcium chloride, 1.44 mM iron (II) sulfate heptahydrate, 4 mM resazurin, 0.1% histidine-hematin, 1% ATCC trace mineral supplement, 1% ATCC vitamin supplement, 29.7 mM acetic acid, 0.9 mM isovaleric acid, 8.1 mM propionic acid, 4.4 mM N-butyric acid with the pH adjusted to 7 using sodium hydroxide. This medium was filter sterilized using a 0.2 um filter and stored in an anaerobic chamber prior to use to allow any dissolved oxygen to dissipate. The single strains of E. coli (BAA-2340, BAA-97, 4 strains isolated from patients, and ECO.139), K. pneumoniae (ATCC 33259, BAA-1705, BAA-2342, and 7 strains isolated from patients), and C. difficile were grown in isolation overnight in CM with 0.5% D-glucose in a COY anaerobic chamber. Single strains of E. faecium (ATCC 700221 and 9 strains isolated from patients, and EFM.70), were grown in isolation overnight in MM with 0.5% D-glucose in a COY anaerobic chamber. 1 mL of each overnight culture was washed with PBS and the optical density (OD₆₀₀) of each culture was measured. Each culture was adjusted to OD₆₀₀ 0.01 in media (e.g., CM or MM).

Inside of the COY anaerobic chamber, the normalized single strain cultures of E. coli, K. pneumoniae, or C. difficile were added to 96 well microplates with one of the oligosaccharide compositions as the sole carbon source in each well. Water added to medium (e.g., CM or MM) without any carbon source functioned as a control. These microplates were then incubated at 37° C. in the COY anaerobic chamber for a total of 45 hours and the OD₆₀₀ was measured every 15 minutes to generate a growth curve for each experimental well. Each oligosaccharide composition was tested in three replicates against each bacterial pathogen.

The area under the curve (AUC) was calculated for the growth curve and a time-to-midlog was determined for each experiment.

A selected oligosaccharide composition did not support the growth (or supported very low growth) of CRE E. coli, CRE K. pneumoniae, VRE E. faecium, or C. difficile. These results further demonstrated that the selected oligosaccharide composition does not support the growth of pathogens, and thereby disadvantages pathogen growth and abundance in microbial communities by selectively favoring the growth of commensal bacteria.

### Example 4. Reduction of Pathogen Growth and Abundance in the Presence of a Selected Oligosaccharide Composition in Cultures of Single Pathogen Strains

A selected oligosaccharide composition comprised of a plurality of oligosaccharides selected from Formula (I), Formula (II), and Formula (III) and produced by a process as described in Examples 7-9 was further tested for its ability to reduce growth and abudance of single strains of pathogens that frequently encountered in critically ill and immunocompromised patients.

Three Nap1 strains of C. difficile and one C. difficile strain from ribotype 012 were obtained from the ATCC® (ATCC® BAA-1870™, ATCC® BAA-1803™, ATCC® BAA-1805™, and ATCC® BAA-1382™). Each strain was grown anaerobically in CM medium at 37° C. for 24 hours until each strain achieved an optical density (OD₆₀₀) of about 1. Each culture was adjusted to an OD₆₀₀ of 0.01 and then incubated with glucose or a sample of the selected oligosacchride composition. Water was added to media without any added carbon source as a negative control. The final concentration of glucose or the selected oligosacchride composition in each assay was 0.5% w/v and each assay was replicated 3 times within each growth plate. Plates were incubated at 37° C. in an anaerobic chamber for a total of 48 hours. Optical density was determined for each strain every 15 minutes for 48 hours.

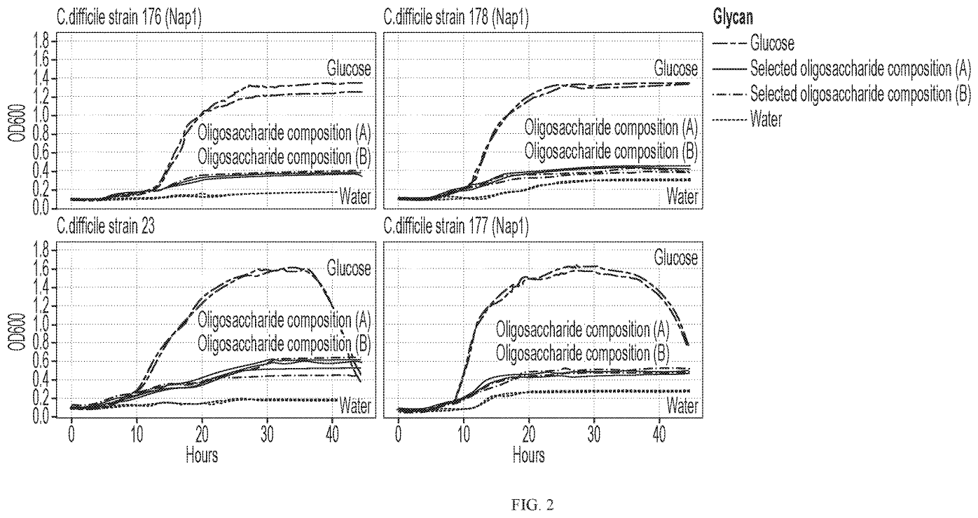

The C. difficile strains tested grew minimally on the oligosaccharide compositions, as did the strains grown in the presence of water (FIG. 2). Meanwhile, each of the C. difficile strains grew to high OD₆₀₀ in the presence of glucose.

The selected oligosaccharide composition was further tested for its ability to reduce the growth and abundance of individual strains of CRE Escherichia coli, CRE Klebsiella pneumoniae, and VRE E. faecium. Single strains of E. coli (one strain obtained from the CDC's Enterobacteriaceae-carbapenem-breakpoint panel, the other isolated from a patient) and K. pneumoniae (one strain from CDC panel, the other isolated from a patient) were grown in isolation overnight in CM medium with 0.5% D-glucose in a COY anaerobic chamber. Single strains of E. faecium (ATCC 700221 and 2 strains isolated from patients) were grown in isolation overnight in MM medium with 0.5% D-glucose in a COY anaerobic chamber. The media was filter sterilized using a 0.2 μm filter and stored in an anaerobic chamber prior to use to allow any dissolved oxygen to dissipate. 1 mL of each overnight culture was washed with PBS and the OD₆₀₀ of each culture was measured. Each culture was adjusted to an OD₆₀₀ of 0.01 and then incubated with glucose, fructooligosaccharide (FOS), or a sample of the selected oligosacchride composition. Water was added to media without any added carbon source as a negative control. The final concentration of glucose, FOS, or the selected oligosacchride composition in each assay was 0.5% w/v and each assay was replicated 3 times within each growth plate. Plates were incubated at 37° C. in an anaerobic chamber for a total of 45 hours. Optical density was determined for each strain every 15 minutes for 48 hours.

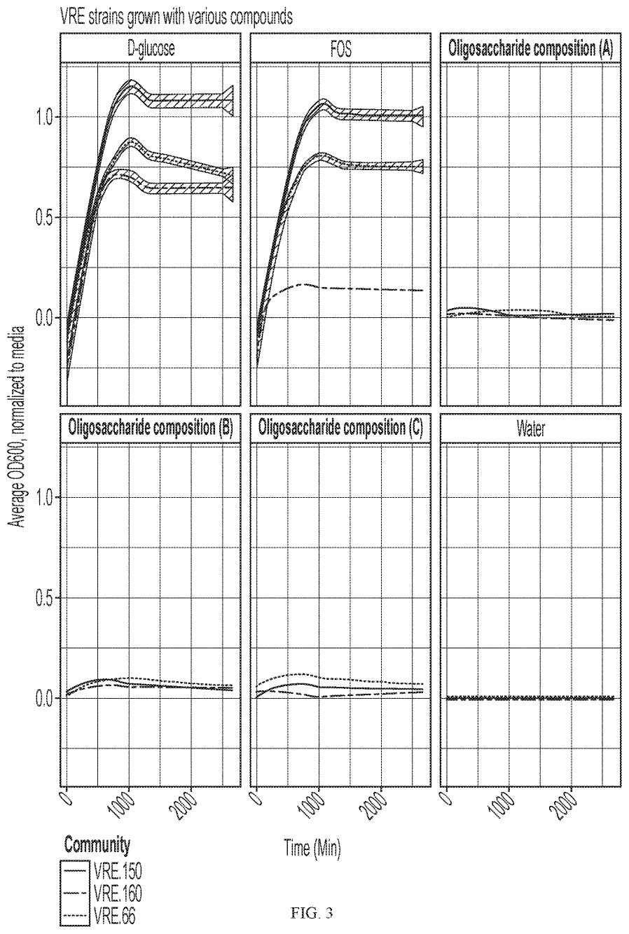

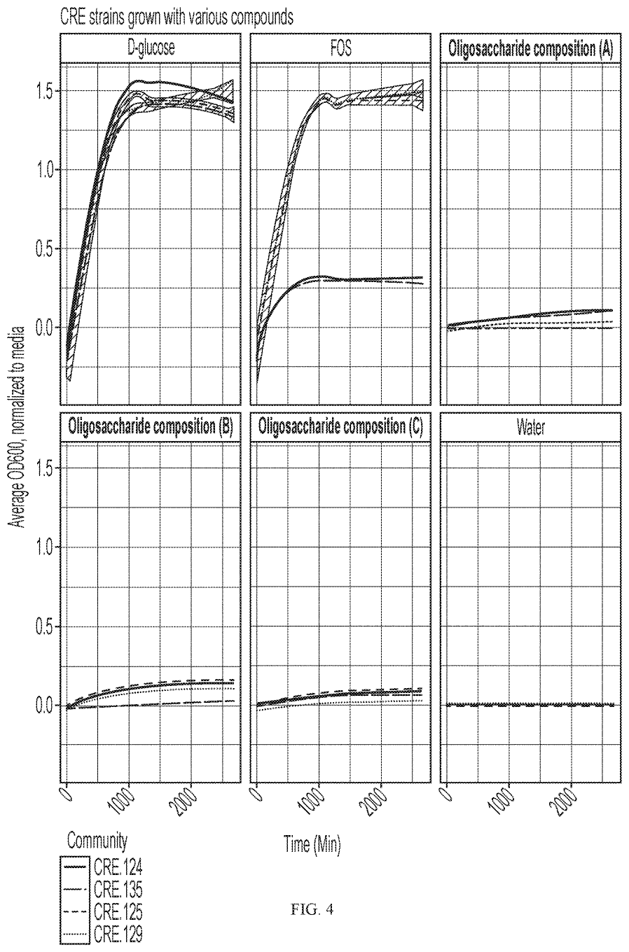

The CRE and VRE pathogens exhibited little-to-no growth in the presence of samples of the selected oligosaccharide composition, similar to the growth of pathogens in the presence of the water control (FIG. 3 and FIG. 4).

The selected oligosaccharide composition was tested for its ability to reduce the growth and abundance of individual strains of fungal pathogens (Candida albicans, Candida glabrata, Candida krusei, and Candida tropicalis). Each of four strains of Candida albicans, Candida glabrata, Candida krusei, and Candida tropicalis were obtained from ATCC (ATCC MYA-2950, ATCC 14243, ATCC 201380 and ATCC MYA-2876). Additional strains of Candida lusitaniae (ATCC 66035 and ATCC 42720) were also tested. All Candida strains were grown aerobically in modified Sabouraud broth (10 g/L peptone solution) with glucose at 2% final concentration at 37° C. for 24 hours until each strain achieved optical density (OD₆₀₀) of about 1. 200 μL of each culture was diluted in 3 mL of modified Sabouraud broth and 120 μL was added to each well of a 96 well plate containing 80 μL of one of the following 5% w/v solutions per well: glucose, FOS, or a sample of the selected oligosaccharide composition. Water was used as a negative control. The final concentration of glucose, FOS, or the selected oligosaccharide composition in each assay to test Candida albicans, Candida glabrata, Candida krusei, or Candida tropicalis was 2%, each assay was replicated 3 times, and plates were incubated at 37° C. for a total of 65 hours. The final concentration of glucose, FOS, or the selected oligosaccharide composition in each assay to test Candida lusitaniae strains was 0.5%, each assay was replicated 3 times, and plates were incubated at 37° C. for a total of 48 hours. Optical density data was collected for each of the Candida albicans, Candida glabrata, Candida krusei, or Candida tropicalis strains every 15 minutes; optical density data was collected for the Candida lusitaniae strains at the end of the experiment.

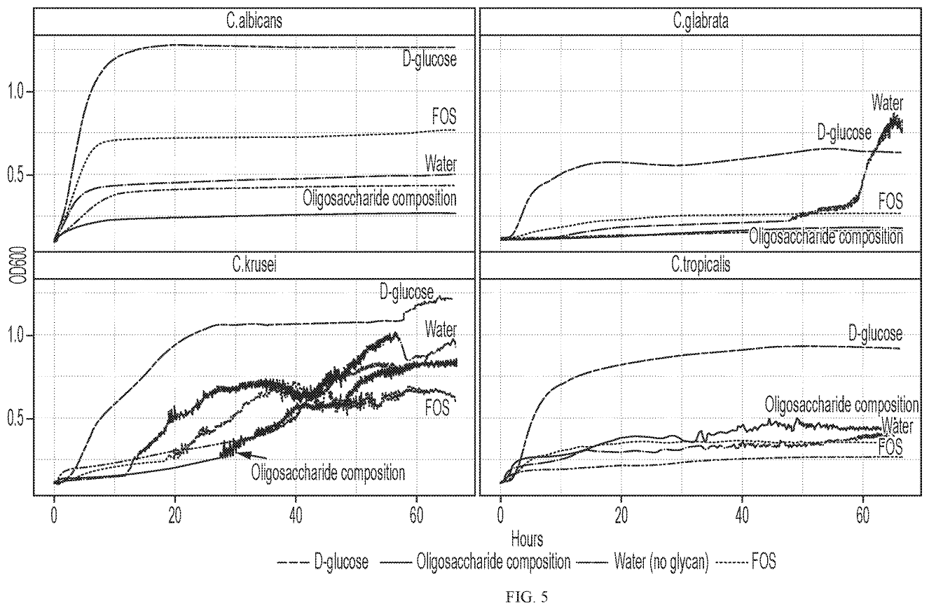

Each of the Candida albicans, Candida glabrata, Candida krusei, or Candida tropicalis strains grew minimally in the presence of the samples of selected oligosaccharide composition (FIG. 5). Meanwhile, each of these strains grew to high OD₆₀₀ in the presence of glucose. Further, growth of each Candida strain in the presence of the selected oligosaccharide composition was similar to the amount of growth in the presence of water (negative control, no carbon source).

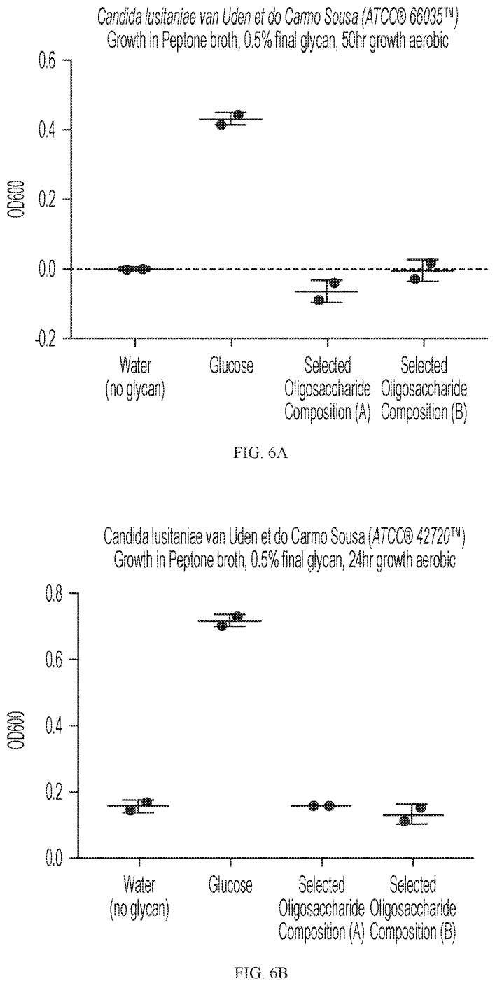

Both of the Candida lusitaniae strains grew minimally in the presence of the samples of selected oligosaccharide composition (FIGS. 6A-6B). Meanwhile, each of these strains grew to high OD₆₀₀ in the presence of glucose. Further, growth of both Candida strains in the presence of the selected oligosaccharide composition was similar to the amount of growth in the presence of water (negative control, no carbon source).

These data collectively demonstrate that the selected oligosaccharide composition as produced according to Examples 7-9 does not support growth and abundance of pathogenic microbes (bacteria and fungi), as evidenced by the inability of any of the tested C. difficile, VRE (E. faecium) and CRE (CRE E. coli, CRE K. pneumoniae), and Candida strains. By contrast, all of the tested strains exhibited significant growth in the presence of glucose and/or FOS.

### Example 5. Assessment of Selected Oligosaccharide Compositions in Fecal Suspensions from Hospitalized Patients

The ability of a selected oligosaccharide composition comprised of a plurality of oligosaccharides selected from Formula (I), Formula (II), and Formula (III) as produced by a process similar to as described in Examples 7-9 to reduce pathogen growth in microbiome samples from fecal suspensions of thirteen hospitalized patients receiving antibiotic treatment from an Intensive Care Unit (ICU) facility was assessed.

Fecal samples from ICU patients and healthy subjects were collected and stored at −80° C. To prepare the fecal material for use in the ex vivo assay, aliquots of a 20% w/v suspension in phosphate buffered saline (PBS) and glycerol were thawed in a COY anaerobic chamber. This suspension was diluted to a final concentration of 1% (w/v) in Mega Medium (MM). The composition of Mega Medium is as described in Romano, K. A. et. al., mBio. 2015 March-April; 6(2): e02481-14. This medium was filter sterilized using a 0.2 μm filter and stored in an anaerobic chamber prior to use to allow any dissolved oxygen to dissipate.

A single strain of Carbapenem-resistant Enterobacteriaceae (CRE) and vancomycin-resistant Enterococcaceae (VRE) were grown in isolation overnight in MM with 0.5% D-glucose in a COY chamber. On the day of the experiment, aliquots of the overnight cultures were washed with PBS and the optical density (OD₆₀₀) of the cultures was measured. The cultures were adjusted to OD₆₀₀ of 0.1 in MM.

The fecal suspensions from ICU patients and healthy subjects were mixed with either of the Carbapenem-resistant Enterobacteriaceae (CRE) culture or the vancomycin-resistant Enterococcaceae (VRE) culture such these pathogens comprised 8% (v/v) of the final mixture. The fecal suspensions were then subjected to 16S metagenomic sequencing to determine the initial abundance (e.g., relative abundance or absolute abundance) of pathogen and commensal bacteria. The cultures were then added to 96-well microplates with one of the following carbon sources (final concentration of 0.5% w/v) in each well: maltodextrin, fructooligosaccharide, a sample of the selected oligosaccharide composition, or water (negative control, i.e., no carbon source). These microplates were then incubated at 37° C. in the COY chamber for a total of 45 hours, with each experimental condition being tested in three replicates on each plate.

At the end of the 45-hour incubation, a sample of the culture from each well was subjected to 16S metagenomic sequencing to determine the final abundance (e.g., relative abundance or absolute abundance) of pathogen and commensal bacteria in the community after intervention with oligosaccharide composition.

For the 16S metagenomic sequencing, genomic DNA was extracted from the fecal suspensions and variable region 4 of the 16S rRNA gene was amplified and sequenced (Earth Microbiome Project protocol www.earthmicrobiome.org/emp-standard-protocols/16s/ and Caporaso J G et al. Ultra-high-throughput microbial community analysis on the Illumina HiSeq and MiSeq platforms. ISME J. (2012) August; 6(8):1621-4). Raw sequences were demultiplexed, and each sample was processed separately with UNOISE2 (Robert Edgar UNOISE2: improved error-correction for Illumina 16S and ITS amplicon sequencing. bioRxiv (2016) Oct. 15). Reads from 16S rRNA amplicon sequencing data were rarefied to 5000 reads, without replacement, and resulting OTU table used in downstream calculations.

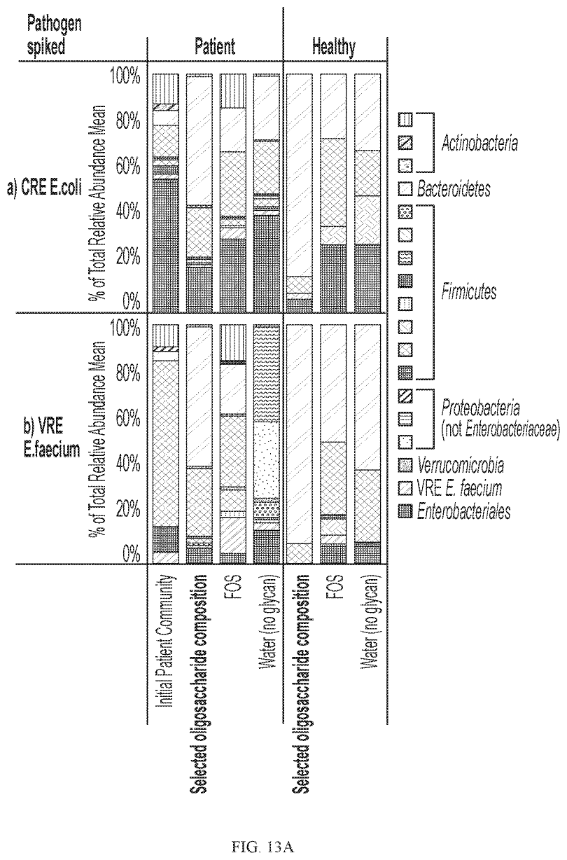

The fecal suspensions from healthy subjects contained a greater diversity in commensal taxa compared to the fecal suspensions from the ICU patients (FIG. 12). For example, the fecal suspensions of three of the thirteen ICU patients contained low levels of commensal bacteria (FIG. 12).

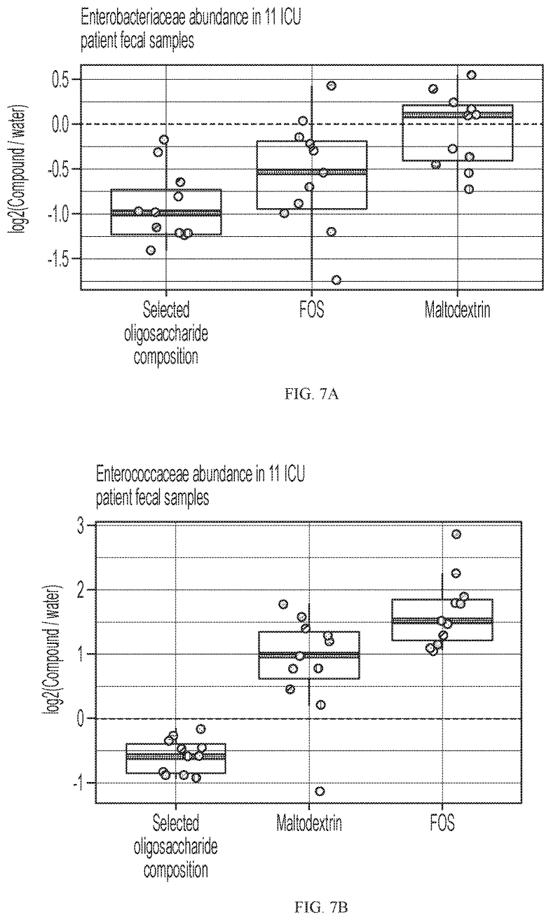

The selected oligosaccharide composition reduced the abundance of Carbapenem-resistant Enterobacteriaceae (FIG. 7A) and vancomycin-resistant Enterococcaceae (FIG. 7B) in spiked fecal suspensions from ICU patients, as assessed by 16S sequencing. A reduction in the abundance (e.g., relative abundance or absolute abundance) of Carbapenem-resistant Enterobacteriaceae was observed in the fecal suspensions from ten of the thirteen ICU patients. There was a smaller reduction in the abundance of Carbapenem-resistant Enterobacteriaceae in the three ICU patients that had few commensal bacteria, indicating that the abundance of commensal bacteria in the gut microbiome can influence the degree of pathogen reduction by the selected oligosaccharide composition. The abundance of each of these pathogens (carbapenem-resistant Enterobacteriaceae and vancomycin-resistant Enterococcaceae) was greater in those spiked fecal suspensions that were incubated in the presence of FOS (a commercial fiber) or maltodextrin. This demonstrates that the selected oligosaccharide composition comprised of a plurality of oligosaccharides selected from Formula (I), Formula (II), and Formula (III) as produced by a process similar to as described in Examples 7-9 is capable of reducing or preventing the growth of pathogens such as Carbapenem-resistant Enterobacteriaceae (CRE) and vancomycin-resistant Enterococcaceae (VRE) in a medically relevant model.

### Example 6. Assessment of Selected Oligosaccharide Compositions in Fecal Suspensions from Hepatic Encephalopathy (HE) Patients

In some cases, pathogen infection could potentially be a precipitating factor of hepatic encephalopathy (HE) in certain patients. Further, HE patients can be immuncompromised and susceptible to pathogen infection. The ability of a selected oligosaccharide composition comprised of a plurality of oligosaccharides selected from Formula (I), Formula (II), and Formula (III) as produced by a process similar to as described in Examples 7-9 to reduce pathogen abundance in patients with HE was testes. Microbiome samples from 44 HE patients were spiked with a single pathogen strain (CRE E. coli or VRE E. faecium) and then grown in the presence of selected oligosaccharide composition, FOS, or water (negative control, i.e., no carbon source).

Fecal samples from HE patients and a healthy subject were collected and stored at −80°. To prepare the fecal material for use in the ex vivo assay, it was moved into a COY anaerobic chamber and made into a 20% w/v suspension in phosphate buffered saline (PBS) with 15% glycerol. Aliquots of each fecal suspension were stored at −80°. For this experiment, an aliquot of each suspension was thawed at ambient temperature within the COY chamber. The aliquots were centrifuged at 2000×g for 5 minutes and the supernatant was discarded. The cell pellet was resuspended in PBS and was then further diluted into a 1% solution in Mega Medium (MM). This medium was filter sterilized using a 0.2 μm filter and stored in an anaerobic chamber prior to use to allow any dissolved oxygen to dissipate.

A single strain of Carbapenem-resistant Enterobacteriaceae (CRE) and vancomycin-resistant Enterococcaceae (VRE) were grown in isolation overnight in MM with 0.5% D-glucose in a COY chamber. On the day of the experiment, aliquots of the overnight cultures were washed with PBS and the optical density (0D600) of the cultures and fecal suspensions were measured. The OD₆₀₀ measurements were used to normalize the bacterial cultures to either an OD₆₀₀ of 0.01 or an OD₆₀₀ of 0.1 and the fecal suspensions to an OD₆₀₀ of 0.1 in MM. After normalization, the CRE strain or VRE strain was added to the fecal suspensions at 8% (v/v) of the total culture. Each batch of CRE strain and VRE strain that was normalized to an OD₆₀₀ of 0.01 was added to 12 of the fecal suspensions. The remaining 32 fecal suspensions were supplemented with cultures of these pathogens that were normalized to an OD₆₀₀ of 0.1. A sample of each pathogen-supplemented fecal suspension was then subjected to shallow shotgun sequencing (16S sequencing) to determine the initial abundance (e.g., relative abundance or absolute abundance) of pathogen and commensal bacteria. The mixed culture was then added to 96-well microplates with one of the following carbon sources (final concentration of 0.5% w/v) in each well: selected oligosaccharide composition, FOS, or water. These microplates were then incubated at 37° C. in the COY chamber for a total of 45 hours. Each oligosaccharide composition was tested in three replicates on each plate, with each experimental condition being tested in three replicates on each plate.

After incubation, 16S sequencing was performed as in Example 5 to determine the abundance of pathogen in each fecal suspension sample.

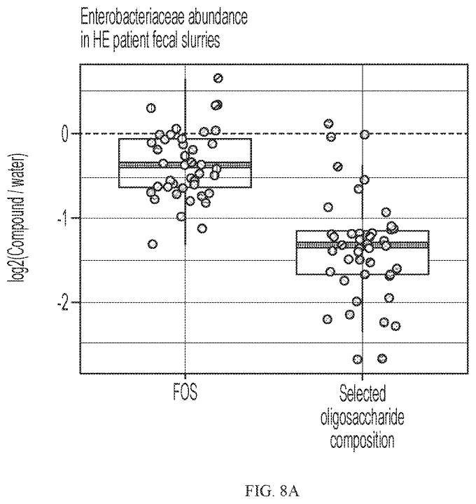

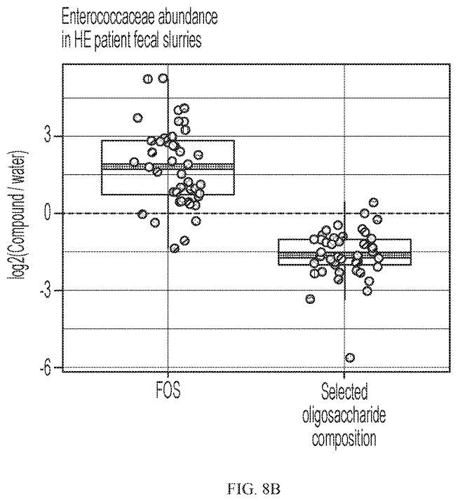

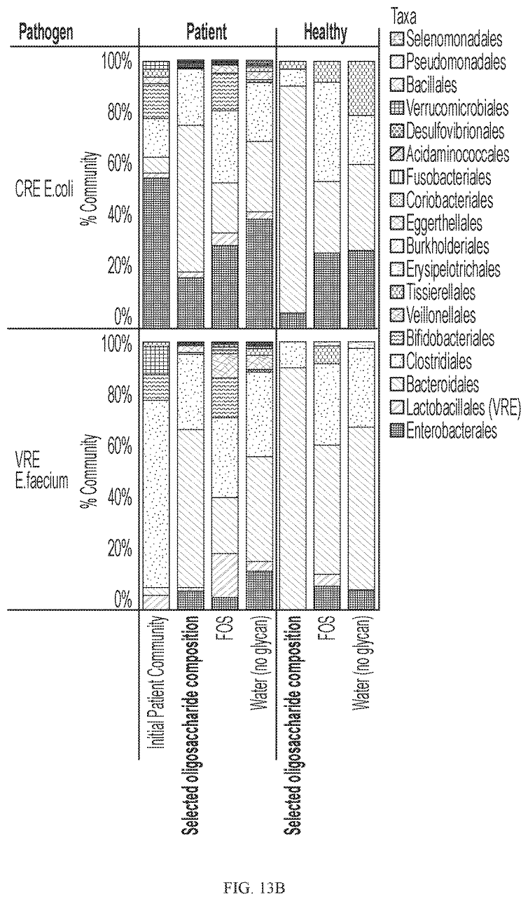

The fold reduction in abundance of CRE pathogen or VRE pathogen for each experimental composition and patient sample was determined relative to water (negative control). The selected oligosaccharide composition reduced the abundance of both Carbapenem-resistant Enterobacteriaceae (FIG. 8A) and vancomycin-resistant Enterococcaceae (FIG. 8B) to a greater degree than FOS, a commercially available fiber. The reduction in the relative abundance of both Carbapenem-resistant Enterobacteriaceae and vancomycin-resistant Enterococcaceae was accompanied by an increase in the abundance of commensal bacteria in the patient fecal suspensions (FIG. 13). For example, there was a decrease in Firmicutes (such as VRE E. faecium and Clostridiales) and an increase in commensal bacteria such as Bacteroidetes (e.g., Bacteroidales) in fecal samples that had been spiked with VRE E. faecium. Additionally, there was a decrease in Proteobacteria (such as Enterobacteriales) and an increase in commensal bacteria such as Bacteroidetes (e.g., Bacteroidales) in fecal samples that had been spiked with CRE E. coli. These results demonstrate that the selected oligosaccharide composition comprised of a plurality of oligosaccharides selected from Formula (I), Formula (II), and Formula (III) as produced by a process similar to as described in Examples 7-9 is capable of reducing or preventing the growth of pathogens such as Carbapenem-resistant Enterobacteriaceae (CRE) and vancomycin-resistant Enterococcaceae (VRE) in a relevant model of hepatic encephalopathy (HE). Further, the selected oligosaccharide composition is capable of inducing the growth and increasing the abundance of commensal microbial species in this relevant model of hepatic encephalopathy (HE).

### Example 7. Production of a Selected Oligosaccharide Composition at 10 kg scale from Dextrose Monohydrate, Galactose and Mannose using a Solid Polymeric Catalyst

A procedure was developed for the synthesis of a selected oligosaccharide composition as described in Examples 4-6 at a 10 kilogram scale. 4.46 kg of dextrose monohydrate, 4.05 kg of galactose, 0.90 kg of mannose, and 0.90 kg (0.450 kg on a dry solid basis) of pre-conditioned solid polymeric acid catalyst (Dowex® Marathon® C resin) were added to a reaction vessel (22 L Littleford-Day horizontal plow mixer) with an attached distillation condenser unit.

The temperature controller was set to 140° C., and stirring (agitation) of the contents of the vessel at 30 RPM was initiated to promote uniform heat transfer and melting of the sugar solids, as the temperature of the syrup was brought to approximately 140° C., under ambient (atmospheric) pressure gradually over a 2.5 hour period.

The reaction mixture was maintained at temperature of approximately 140° C. for 1.5 hours (90 min), after which the heating was stopped and pre-heated water was gradually added to the reaction mixture at a rate of 60 mL/min until the temperature of the reactor contents decreased to 120° C., then at a rate of 150 mL/min until the temperature of the reactor contents decreased to 110° C., and then at a rate of 480 mL/min until the temperature of the reactor contents decreased below 100° C. and a total of 6 kg of water was added. An additional 1.75 kg of water was added to the reactor for further dilution.

The reaction mixture was drained from the vessel and the solids were removed by filtration, resulting in 15 kg of crude oligosaccharide composition product material as an aqueous solution (approximately 45 wt %).

The oligosaccharide composition composition was purified by flowing it through a cationic exchange resin (Dowex® Monosphere® 88H) column, two columns of decolorizing polymer resin (Dowex® OptiPore® SD-2), and an anionic exchange resin (Dowex® Monosphere® 77WBA) column. The resulting purified oligosaccharide composition had a concentration of about 35 wt % and was then concentrated to a final concentration of about 75 wt % solids by vacuum rotary evaporation.

### Example 8. Production of Oligosaccharide Composition at 100 g Scale from Dextrose Monohydrate, Galactose and Mannose using a Solid Polymeric Catalyst

A procedure was developed for the synthesis of a selected oligosaccharide composition as described in Examples 4-6 at a 100 gram scale. 45 g of dextrose monohydrate, 45 g of galactose, 10 g of mannose were added to a reaction vessel (1 L three-neck round-bottom flask). The reaction vessel was equipped with a heating mantle configured with an overhead stirrer. A probe thermocouple was disposed in the vessel through a septum, such that the probe tip sat above the stir blade and not in contact with the walls of the reaction vessel. Prior to addition of catalyst, the reaction vessel was equipped with a condenser in a reflux position.

The procedure also used an acidic oligomerization catalyst (Dowex Marathon C) (3-5% w/w) and de-ionized water for quenching. In some cases, the catalyst was handled in wet form, e.g., at a nominal moisture content of 45-50 wt % H₂O. The exact catalyst moisture content was generally determined on a per-experiment basis using, for example, using a moisture analyzing balance (e.g., Mettler-Toledo MJ-33). Following addition of catalyst, the reaction vessel was equipped in a distillation position to remove excess water throughout the course of the reaction.

The temperature controller was set to a target temperature (100 to 160° C.), and stirring of the contents of the vessel was initiated to promote uniform heat transfer and melting of the sugar solids, as the temperature of the syrup was brought to the target temperature, under ambient (atmospheric) pressure.

Upon addition of the catalyst, the reaction was maintained at the target temperature under continuous mixing for about 4 hours, determined by following the reaction by HPLC. Next, the heat was turned off while maintaining constant stirring.

The reaction was then quenched by slowly adding approximately 60 mL of deionized (DI) water (room temperature) to dilute and cool the product mixture, to target a final concentration of 60-70 wt % dissolved solids. Generally, the water addition rate was performed to control the mixture viscosity as the oligosaccharide composition was cooled and diluted.

Following dilution, the oligosaccharide composition was cooled to approximately 60° C. The catalyst was then removed by vacuum filtration through a 100 micron mesh screen or fritted-glass filter, to obtain the final oligosaccharide composition at around 40° Bx.

### Example 9. Production of the Selected Oligosaccharide Composition at 10 kg Scale from Dextrose Monohydrate, Galactose and Mannose using a Citric Acid Catalyst

A procedure was developed for the synthesis of the selected oligosaccharide composition as described in Examples 4-6 at a 10 kilogram scale. 4.46 kg of dextrose monohydrate, 4.05 kg of galactose, 0.90 kg of mannose, 0.29 kg citric acid monohydrate acid catalyst (or 0.27 kg citric acid anhydrous) and 0.48 kg water were added to a reaction vessel (22 L Littleford-Day horizontal plow mixer). A distillation condenser unit was attached to the reactor.

The contents were agitated at approximately 30 RPM and the vessel temperature was gradually increased over a 2.5 hour period to about 139° C. at atmospheric pressure. The mixture was maintained at temperature for one and half hours. The heating was subsequently stopped and pre-heated water was gradually added to the reaction mixture at a rate of 60 mL/min until the temperature of the reactor contents decreased to 120° C., then at 150 mL/min until the temperature of the reactor contents decreased to 110° C., then at 480 mL/min until a total of 6 kg of water was added, and the temperature of the reactor contents decreased below 100° C. An additional 1.75 kg water was added to the reactor for further dilution. The reaction mixture was drained from the vessel, resulting in 15 kg of crude oligosaccharide composition product as an aqueous solution (approximately 53 wt %).

### Example 10. De-Monomerization Procedure

Individual batches of oligosaccharide composition, as produced in Examples 7-9 were concentrated on a rotatory evaporator to approximately 50 Brix as measured by a Brix refractometer following treatment with ion-exchange resins (e.g., as described herein). The resulting syrup (200 mg) was loaded onto a Teledyne ISCO RediSep Rf Gold Amine column (11 grams stationary phase) using a luer-tip syringe. Other similar columns such as the Biotage SNAP KP-NH Catridges may also be used. The sample was purified on a Biotage Isolera equipped with an ELSD detector using a 20/80 to 50/50 (v/v) deionized water/ACN mobile phase gradient over 55 column volumes. Other flash chromatography systems such as the Teledyne ISCO Rf may also be used. The flow rate was set in accordance with the manufacturer's specifications for the column and system. After the monomer fraction completely eluted at ˜20 column volumes, the mobile phase was set to 100% water until the remainder of the oligosaccharide composition eluted and was collected. The non-monomer containing fractions were concentrated by rotary evaporation to afford the de-monomerized product.

### Example 11. Collection of Fecal Samples

Fecal samples were collected by providing subjects with the Fisherbrand Commode Specimen Collection System (Fisher Scientific) and associated instructions for use. Collected samples were stored with ice packs or at −80° C. until processing (McInnes & Cutting, Manual of Procedures for Human Microbiome Project: Core Microbiome Sampling Protocol A, 2010, hmpdacc.org/doc/HMP_MOP_Version12_0_072910.pdf). Alternative collection devices may also be used. For example, samples may be collected into the Faeces Tube 54×28 mm (Sarstedt AG, 25 ml SC Feces Container w/Scoop), Globe Scientific Screw Cap Container with Spoon (Fisher Scientific) or the OMNIgene-GUT collection system (DNA Genotek, Inc.), which stabilizes microbial DNA for downstream nucleic acid extraction and analysis. Aliquots of fecal samples were stored at -20° C. and -80° C. following standard protocols known to one skilled in the art.

### Example 12. Determining the Level of Pathogens in Subjects

To determine the titer of pathogens carried in the gastrointestinal tract, fecal samples or rectal swabs are collected by a suitable method. Sample material is cultured on, e.g., i) Cycloserine-Cefoxitin Fructose Agar (available for instance from Anaerobe Systems) cultured anaerobically to selectively and differentially grow Clostridium difficile; ii) Eosin Methylene Blue Agar (available for instance from Teknova) cultured aerobically to titer Escherichia coli and other Gram-negative enteric bacteria, most of which are opportunistic pathogens; iii) Bile Esculin Agar (BD) cultured aerobically to titer Enterococcus species; iv) phenyl-ethylalcohol blood agar (Becton Dickinson), or Colistin-Nalidixic Acid (CNA) blood agar (for instance, from Hardy Diagnostics) cultured aerobically to grow Enterococcus and/or Streptococcus species; v) Bifidobacterium Selective Agar (Anaerobe Systems) to titer Bifidobacterium species; vi) or MacConkey Agar (Fisher Scientific) to titer E. coli and other Gram-negative enteric bacteria. Additional antibiotics can be used as appropriate to select drug-resistant subsets of these bacteria, for instance vancomycin (e.g., for vancomycin-resistant Enterococcus), cefoxitin (e.g., for extended spectrum beta lactamases or Enterococcus), ciprofloxacin (e.g., for fluoroquinolone resistance), ampicillin (e.g., for ampicillin resistant bacteria), and ceftazidime (e.g., for cephalosporin resistant bacteria). Additionally, chromogenic substrates may be added to facilitate the differentiation of pathogens from commensal strains, such as with ChromID plates (Biomerieux) or ChromAgar (Becton Dickinson). Plates are incubated at 35-37° C. under aerobic, anaerobic or microaerophilic conditions as appropriate for the pathogen. After 16-48 hours, colonies are counted and used to back-calculate the concentration of viable cells in the original sample.

For quantitative assessment, the subjects sample volume or weight is measured, and serial 1:10 dilutions prepared in phosphate buffered saline or other diluent, followed by plating, growth and counting of colonies to determine the level of a pathogen in a sample.

Alternatively, the quantity of a pathogen is measured by quantitative PCR. For this method, primers specific to one or more of the pathogens (including bacterial pathogens, viral pathogens and pathogenic protozoa) described herein are designed and used in a real-time quantitative PCR (for instance, using a PCR reaction to which a double-stranded-specific fluorescent dye such as Sybr Green, or a sequence-specific Taqman probe (Applied Biosystems/Thermo Scientific). Genomic DNA is extracted from each sample using the Mo Bio Powersoil®-htp 96 Well Soil DNA Isolation Kit (Mo Bio Laboratories, Carlsbad, Calif.) according to the manufacturer's instructions or by bead beating, e.g., performed for 2 minutes using a BioSpec Mini-Beadbeater-96 (BioSpec Products, Bartlesville, Okla.). Alternatively, the genomic DNA is isolated using the Mo Bio Powersoil® DNA Isolation Kit (Mo Bio Laboratories, Carlsbad, Calif.) or the QIAamp DNA Stool Mini Kit (QIAGEN, Valencia, Calif.) according to the manufacturer's instructions. The cycle threshold of a sample of a subject in quantitative PCR is then compared to a standard curve of known quantities of pathogens to determine the level of pathogen in the sample. The development of assays is described (e.g., in “Application of the fluorogenic probe technique (TaqMan PCR) to the detection of Enterococcus spp. and Escherichia coli in water samples”, Edith Frahm and Ursala Obst, J. Microbiol. Meth. 2003 January; 52(1):123-31.). Alternatively, to simplify assay design, analyte-specific reagents are available for many of the pathogens, for instance from Luminex, Inc (www.luminexcorp.com). Alternatively, or in addition, universal ribosomal primers are used to quantitatively measure the total copy number of genomes from pathogens to determine relative instead of absolute abundance of pathogens. If desired, the ratio of pathogen to total copies is calculated. The colony counts can be normalized (e.g., a ratio is calculated) to the total DNA content of the sample, or to the quantitative measure, e.g., determined by a qPCR using universal ribosomal primers.

Alternatively, the colony count of a pathogen, or all pathogens combined, is compared to the total colony count of the sample cultured under non-selective conditions. Samples are cultured on rich media or agar such as Brucella Blood Agar (Anaerobe Systems), Brain Heart Infusion Broth (Teknova), or chocolate agar (Anaerobe Systems). The maximum number of colonies on these media, grown anaerobically are used as the denominator in a normalized ratio of pathogens to commensals as a relative measure.

The amount of pathogen may also be estimated by 16s ribosomal DNA profiling. Genomic DNA is extracted from subject samples (e.g. fecal samples, rectal swabs, skin or mucosal swabs, biopsies or tissue samples), and variable region 4 of the 16S rRNA gene is amplified and sequenced (Earth Microbiome Project protocol www.earthmicrobiome.org/emp-standard-protocols/16s/ and Caporaso JG et al. 2012. Ultra-high-throughput microbial community analysis on the Illumina HiSeq and MiSeq platforms. ISME J.). Operational Taxonomic Units (OTUs) are generated by aligning 16S rRNA sequences at 97% identity, or lower as appropriate. Then the OTUs potentially representing pathogenic species are assessed by aligning the OTUs to known taxonomic structures such as those maintained by NCBI (ncbi.nlm.nih.gov) or the Ribosomal Database Project (https://rdp.cme.msu.edu), and their abundance estimated, for instance as a ratio of number of pathogen sequences to total number of sequences.

### Example 13. Determination of Glycosidic Bond Distribution using Permethylation Analysis

A determination of glycosidic bond distribution of samples of the selected oligosaccharide composition, as produced by the process in Examples 7 and 9, was performed using permethylation analysis, according to the protocol described below. Samples were demonomerized prior to permethylation analysis.

Reagents used were methanol, acetic acid, sodium borodeuteride, sodium carbonate, dichioromethane, isopropanol, trifluoroacetic acid (TFA), and acetic anhydride. Equipment included a heating block, drying apparatus, gas chromatograph equipped for capillary columns and with a RID/MSD detector, and a 30 meter RTX®-2330 (RESTEK). All derivation procedures were done in a hood.

### Preparation of Alditol Acetates

### A. Standard Preparation

1 mg/mL solutions of the following standard analytes were prepared: arabinose, rhamnose, fucose, xylose, mannose, galactose, glucose, and inositol. The standard was prepared by mixing 50 μL of each of arabinose, xylose, fucose, glucose, mannose, and galactose with 20 μL of inositol in a vial. The standard was subsequently lyophilized.

### B. Sample Preparation

Each sample was prepared by mixing 100-500 μL of of the selected oligosaccharide composition (as weighed on an analytical balance) with 20 μL (20 μL) of inositol in a vial.

### C. Hydrolysis

200 μL of 2 M tifluoroacetic acid (TFA) was added to the sample(s). The vial containing the sample was capped tightly and incubated on a heating block for 2 hours at 121° C. After 2 hours, the sample was removed from the heating block and allowed to cool to room temperature. The sample was then dried down with N₂/air. 200 μL of IPA (isopropanol) was added and dried down again with N₂/air. This hydrolysis step (addition of TFA for two hours at 121° C.; washing with isopropanol) was repeated twice.

The standard was similarly subjected to hydrolysis using TFA, as described for the sample.

### D. Reduction and Acetylation

10 mg/mL solution of sodium borodeuteride was prepared in 1 M ammonium hydroxide. 200 μL of this solution was added to the sample. The sample was then incubated at room temperature for at least one hour or overnight. After incubation with sodium borodeuteride solution, 5 drops of glacial acetic acid were added to the sample, followed by 5 drops of methanol. The sample was then dried down. 500 μL of 9:1 MeOH:HOAc was added to the sample and subsequently dried down (twice repeated). 500 μL MeOH was then added to the sample and subsequently dried down (once repeated). This produced a crusty white residue on the side of the sample vial.

250 μL acetic anhydride was then added to the sample vial and the sample was vortexed to dissolve. 230 μL concentrated TFA was added to the sample and the sample was incubated at 50° C. for 20 minutes. The sample was removed from the heat and allowed to cool to room temperature. Approximately 1 mL isopropanol was added and the sample was dried down. Then, approximately 200 μL isopropanol was added and the sample was dried down again. Approximately 1 mL of 0.2M sodium carbonate was then added to the sample and it was mixed gently. Approximately 2 mL dichloromethane was finally added to the sample, after which it was vortexed and centrifuged briefly. The aqueous top layer was discarded. 1 mL water was added and the sample was vortexed and centrifuged briefly. This step was repeated before the organic layer (bottom) was removed and transferred to another vial. The sample was concentrated using N₂/air to a final volume of about 100 μL. 1 μL of final sample was then injected on GC-MS.

The GC temperature program SP2330 was utilized for GC-MS analysis. The initial temperature was 80° C. and the initial time was 2.0 minutes. The first ramp was at a rate of 30° C./min with a final temperature of 170° C. and a final time of 0.0 minutes. The second ramp was at a rate of 4° C./min with a final temperature of 240° C. and a final time of 20.0 minutes.

### Glycosyl-Linkage Analysis of Poly- and Oligosaccharides by Hakomori Methylation

### A. Preparation of NaOH Base

In a glass screw top tube, 100 μL of a 50/50 NaOH solution and 200 μL of dry MeOH were combined. Plastic pipets were used for the NaOH and glass pipets were used for the MeOH. The solution was vortexed briefly, approximately 4 mL dry DMSO was added, and the solution was vortexed again. The tube was centrifuged to concentrate the solution and the DMSO and salts were pipetted off from the pellet. The previous two steps were repeated about four times in order to remove all the water from the pellet. All white reside was removed from the sides of the tube. Once all the residue was removed and the pellet was clear, about 1 mL dry DMSO was added and the solution was vortexed. The base was then ready to use. The base was prepared fresh each time it was needed.

### B. Permethylation

Each sample was prepared by mixing 600-1000 μg of the selected oligosaccharide composition (as weighed on an analytical balance) with 200 μL DMSO. The sample was stirred overnight until the oligosaccharide composition dissolved.

An equal amount of NaOH base (400 μL) was added to the sample, after which the sample was placed back on the stirrer and mixed well for 10 minutes. 100 μL of iodomethane (CH₃I) was added to the sample. The sample was mixed on the stirrer for 20 minutes, and then the previous steps (addition of NaOH base and iodomethane) were repeated.

Approximately 2 mL ultrapure water was added to the sample and the sample was mixed well, such that it turned cloudy. The tip of a pipette was placed into the sample solution at the bottom of the tube and CH₃I was bubbled off with a very low flow of air. The sample became clear as the CH₃I was bubbled off. The pipette was moved around the solution to make certain that all the CH₃I was gone. Approximately 2 mL methylene chloride was then added and the solution was mixed well by vortex for 30 seconds. The sample was then centrifuged and the top aqueous layer was removed. Approximately 2 mL of water were added and the sample was mixed, then briefly centrifuged, then the top aqueous layer was removed. The additions of methylene chloride and water were repeated. The organic bottom layer was removed and transferred into another tube and dried down using N₂. The analysis was continued with Alditol Acetates.

### C. Hydrolysis

200 μL of 2 M tifluoroacetic acid (TFA) was added to the sample(s). The vial containing the sample was capped tightly and incubated on a heating block for 2 hours at 121° C. After 2 hours, the sample was removed from the heating block and allowed to cool to room temperature. The sample was then dried down with N₂/air. 200 μL of IPA (isopropanol) was added and dried down again with N₂/air. This hydrolysis step (addition of TFA for two hours at 121° C.; washing with isopropanol) was repeated twice.

### D. Reduction and Acetylation

10 mg/mL solution of sodium borodeuteride was prepared in 1 M ammonium hydroxide. 200 μL of this solution was added to the sample. The sample was then incubated at room temperature for at least one hour or overnight. After incubation with sodium borodeuteride solution, 5 drops of glacial acetic acid were added to the sample, followed by 5 drops of methanol. The sample was then dried down. 500 μL of 9:1 MeOH:HOAc was added to the sample and subsequently dried down (twice repeated). 500 μL MeOH was then added to the sample and subsequently dried down (once repeated). This produced a crusty white residue on the side of the sample vial.

250 μL acetic anhydride was then added to the sample vial and the sample was vortexed to dissolve. 230 μL concentrated TFA was added to the sample and the sample was incubated at 50° C. for 20 minutes. The sample was removed from the heat and allowed to cool to room temperature. Approximately 1 mL isopropanol was added and the sample was dried down. Then, approximately 200 μL isopropanol was added and the sample was dried down again. Approximately 1 mL of 0.2M sodium carbonate was then added to the sample and it was mixed gently. Approximately 2 mL dichloromethane was finally added to the sample, after which it was vortexed and centrifuged briefly. The aqueous top layer was discarded. 1 mL water was added and the sample was vortexed and centrifuged briefly. This step was repeated before the organic layer (bottom) was removed and transferred to another vial. The sample was concentrated using N₂/air to a final volume of about 100 μL. 1 μL of final sample was then injected on GC-MS.

The GC temperature program SP2330 was utilized for GC-MS analysis. The initial temperature was 80° C. and the initial time was 2.0 minutes. The first ramp was at a rate of 30° C./min with a final temperature of 170° C. and a final time of 0.0 minutes. The second ramp was at a rate of 4° C./min with a final temperature of 240° C. and a final time of 20.0 minutes.

Results

Permethylation data was collected using the methods described above for four batches of de-monomerized oligosaccharide composition produced by the process described in Example 7 (IIIarathon C catalyst). Each batch was analyzed in duplicate. Averaged data relating to the radicals present in these ten batches of de-monomerized oligosaccharide composition are provided below:

| | Mean mol | | Mean mol |
|---|---|---|---|
| | % +3 | Mean mol | % −3 |
| Radicals | STD | % | STD |
| t-mannopyranose | 4.10% | 3.56% | 3.02% |
| t-glucopyranose | 16.33% | 13.89% | 11.44% |
| t-galactofuranose | 7.78% | 4.52% | 1.26% |
| t-glucofuranose | 1.38% | 0.64% | 0.00% |
| t-galactopyranose | 12.48% | 10.38% | 8.29% |
| 3-glucopyranose | 4.88% | 3.95% | 3.02% |
| 2-mannopyranose and/or | 1.94% | 1.57% | 1.20% |
| 3-mannopyranose | | | |
| 2-glucopyranose | 3.22% | 2.83% | 2.44% |
| 2-galactofuranose and/or | 2.32% | 1.62% | 0.93% |
| 2-glucofuranose | | | |
| 3-galactopyranose | 3.92% | 3.43% | 2.94% |
| 4- mannopyranose and/or | 2.93% | 2.34% | 1.75% |
| 5-mannofuranose and/or | | | |
| 3 -galactofuranose | | | |
| 6-mannopyranose | 2.87% | 2.44% | 2.01% |
| 2-galactopyranose | 2.71% | 2.28% | 1.85% |
| 6-glucopyranose | 10.78% | 9.22% | 7.66% |
| 4-galactopyranose and/or | 3.80% | 3.22% | 2.65% |
| 5-galactofuranose | | | |
| 4-glucopyranose and/or | 4.25% | 3.66% | 3.06% |
| 5-glucofuranose and/or | | | |
| 6-mannofuranose | | | |
| 6-glucofuranose | 1.55% | 0.81% | 0.08% |
| 6-galactofuranose | 4.96% | 3.19% | 1.42% |
| 6-galactopyranose | 9.06% | 7.44% | 5.81% |
| 3,4-galactopyranose and/or | 1.42% | 1.16% | 0.90% |
| 3,5-galactofuranose and/or | | | |
| 2,3-galactopyranose | | | |
| 3,4-glucopyranose and/or | 1.04% | 0.43% | 0.00% |
| 3,5-glucofuranose | | | |
| 2,4-glucopyranose and/or | 1.39% | 1.16% | 0.92% |
| 2,5-glucofuranose and/or | | | |
| 2,4-galactopyranose and/or | | | |
| 2,5-galactofuranose | | | |
| 4,6-mannopyranose and/or | 0.69% | 0.59% | 0.49% |
| 5,6-mannofuranose | | | |
| 3,6-mannofuranose | 0.11% | 0.02% | 0.00% |
| 3,6-glucopyranose | 2.80% | 2.10% | 1.40% |
| 3,6-mannopyranose and/or | 0.67% | 0.53% | 0.39% |
| 2,6-mannofuranose | | | |
| 2,6-mannopyranose | 0.54% | 0.41% | 0.28% |
| 3,6-glucofuranose | 0.39% | 0.27% | 0.16% |
| 2,6-glucopyranose and/or | 3.58% | 2.33% | 1.08% |
| 4,6-glucopyranose and/or | | | |
| 5,6-glucofuranose | | | |
| 3,6-galactofuranose | 1.37% | 1.15% | 0.93% |
| 4,6-galactopyranose and/or | 2.86% | 2.48% | 2.11% |
| 5,6-galactofuranose | | | |
| 3,6-galactopyranose and/or | 2.98% | 2.28% | 1.58% |
| 2,6-galactofuranose | | | |
| 2,6-galactopyranose | 1.62% | 1.15% | 0.68% |
| 3,4,6-mannopyranose and/or | 0.30% | 0.07% | 0.00% |
| 3,5,6-mannofuranose and/or | | | |
| 2,3,6-mannofuranose | | | |
| 3,4,6-galactopyranose and/or | 1.11% | 0.82% | 0.53% |
| 3,5,6-galactofuranose and/or | | | |
| 2,3,6-galactofuranose | | | |
| 3,4,6-glucopyranose and/or | 0.47% | 0.35% | 0.22% |
| 3,5,6-glucofuranose | | | |
| 2,3,6-mannopyranose and/or | 0.49% | 0.17% | 0.00% |
| 2,4,6-mannopyranose and/or | | | |
| 2,5,6-mannofuranose | | | |
| 2,4,6-glucopyranose and/or | 1.36% | 0.56% | 0.00% |
| 2,5,6-glucofuranose | | | |
| 2,3,6-galactopyranose and/or | 0.91% | 0.66% | 0.41% |
| 2,4,6-galactopyranose and/or | | | |
| 2,5,6-galactofuranose | | | |
| 2,3,6-glucopyranose | 0.48% | 0.31% | 0.13% |

Permethylation data was collected using the methods described above for five batches of de-monomerized oligosaccharide composition produced by the process described in Example 9 (citric acid catalyst). Each batch was analyzed in duplicate. Averaged data relating to the radicals present in these ten batches of de-monomerized oligosaccharide composition are provided below:

| | Mean mol | | Mean mol |
|---|---|---|---|
| | % +3 | Mean mol | % −3 |
| Radicals | STD | % | STD |
| t-mannopyranose | 4.14% | 3.57% | 3.00% |
| t-glucopyranose | 17.59% | 15.58% | 13.58% |
| t-galactofuranose | 4.20% | 3.59% | 2.98% |
| t-glucofuranose | 0.73% | 0.15% | 0.00% |
| t-galactopyranose | 11.69% | 10.67% | 9.65% |
| 3-glucopyranose | 4.61% | 4.22% | 3.84% |
| 2-mannopyranose and/or | 1.99% | 1.41% | 0.83% |
| 3-mannopyranose | | | |
| 2-glucopyranose | 3.03% | 2.88% | 2.72% |
| 2-galactofuranose and/or | 1.78% | 1.30% | 0.83% |
| 2-glucofuranose and/or | | | |
| 3-glucofuranose | | | |
| 3-galactopyranose | 3.77% | 3.28% | 2.79% |
| 3 -galactofuranose | 2.24% | 1.92% | 1.60% |
| 6-mannopyranose | 2.47% | 2.28% | 2.08% |
| 2-galactopyranose | 2.42% | 2.03% | 1.65% |
| 6-glucopyranose | 11.06% | 10.29% | 9.53% |
| 4-galactopyranose and/or | 3.06% | 2.75% | 2.45% |
| 5-galactofuranose | | | |
| 4-glucopyranose and/or | 3.89% | 3.45% | 3.00% |
| 5-glucofuranose and/or | | | |
| 6-mannofuranose | | | |
| 2,3-galactofuranose | 0.42% | 0.05% | 0.00% |
| 6-glucofuranose | 0.77% | 0.31% | 0.00% |
| 6-galactofuranose | 2.68% | 2.50% | 2.31% |
| 6-galactopyranose | 8.75% | 7.90% | 7.06% |
| 3,4-galactopyranose and/or | 1.08% | 0.97% | 0.86% |
| 3,5-galactofuranose and/or | | | |
| 2,3-galactopyranose | | | |
| 3,4-glucopyranose and/or | 0.75% | 0.61% | 0.47% |
| 3,5-glucofuranose | | | |
| 2,3-glucopyranose | 2.11% | 0.89% | 0.00% |
| 2,4-mannopyranose and/or | 0.85% | 0.21% | 0.00% |
| 2,5-mannofuranose | | | |
| 2,4-glucopyranose and/or | 1.88% | 1.17% | 0.45% |
| 2,5-glucofuranose and/or | | | |
| 2,4-galactopyranose and/or | | | |
| 2,5-galactofuranose | | | |
| 4,6-mannopyranose and/or | 0.70% | 0.53% | 0.35% |
| 5,6-mannofuranose | | | |
| 3,6-glucopyranose | 2.93% | 2.47% | 2.02% |
| 3,6-mannopyranose | 0.66% | 0.54% | 0.43% |
| 2,6-mannopyranose | 0.51% | 0.45% | 0.39% |
| 3,6-glucofuranose | 0.33% | 0.12% | 0.00% |
| 2,6-glucopyranose and/or | 2.55% | 2.15% | 1.74% |
| 4,6-glucopyranose and/or | | | |
| 5,6-glucofuranose | | | |
| 3,6-galactofuranose | 1.16% | 1.01% | 0.86% |
| 4,6-galactopyranose and/or | 2.86% | 2.47% | 2.09% |
| 5,6-galactofuranose | | | |
| 3,6-galactopyranose | 2.73% | 2.36% | 1.99% |
| 2,6-galactopyranose | 1.49% | 1.24% | 0.99% |
| 3,4,6-mannopyranose and/or | 0.12% | 0.01% | 0.00% |
| 3,5,6-mannofuranose and/or | | | |
| 2,3,6-mannofuranose | | | |
| 3,4,6-galactopyranose and/or | 0.96% | 0.75% | 0.54% |
| 3,5,6-galactofuranose and/or | | | |
| 2,3,6-galactofuranose | | | |
| 3,4,6-glucopyranose and/or | 0.63% | 0.28% | 0.00% |
| 3,5,6-glucofuranose | | | |
| 2,3,6-mannopyranose | 0.34% | 0.12% | 0.00% |
| 2,4,6-glucopyranose and/or | 0.80% | 0.40% | 0.00% |
| 2,5,6-glucofuranose | | | |
| 2,3,6-galactopyranose and/or | 1.31% | 0.59% | 0.00% |
| 2,4,6-galactopyranose and/or | | | |
| 2,5,6-galactofuranose | | | |
| 2,4,6-galactopyranose and/or | 0.92% | 0.15% | 0.00% |
| 2,5,6-galactofuranose | | | |
| 2,3,6-glucopyranose | 0.74% | 0.37% | 0.00% |

### Example 14. HSQC NMR Analysis Procedure using a Varian Unity Inova NMR Machine

A determination of HSQC NMR spectra of samples of the selected oligosaccharide composition, as produced by the processes in Examples 7 and 9, were performed using a Varian Unity Inova NMR, according to the protocol described below.

Method

Sample Preparation:

25 mg of a previously lyophilized solid sample was dissolved in 300 uL of D2O with 0.1% acetone as internal standard. The solution was then placed into a 3 mm NMR tube.

NMR Experiment:

The sample was analyzed in a Varian Unity Inova operating at 499.83 MHz (125.69 MHz 13C) equipped with a XDB broadband probe with Z-axis gradient, tuned to 13C, and operating at 25° C. The sample was subjected to a heteroatomic single quantum coherence (HSQC), echo-antiecho, with gradient selection HSQCETGP pulse sequence experiment using the following acquisition and processing parameters in Table 3:

TABLE 3
Acquisition Parameter

| | |
|---|---|
| Number of Scans | 8 |
| Recycle Delay | 1 second |
| Number of datapoints | 596 × 600 |
| Sweep Width (ppm) | 4 ppm × 110 ppm |
| Carrier Frequency | 4.0 ppm-65 ppm |
| CH Coupling Constant | 146 Hz |

Processing Parameter

| | |
|---|---|
| Size of Matrix | 1024 × 2048 |
| Window Function | Gaussian (7.66, 26.48 Hz) |
| Baseline correction | No correction |

Spectral Analysis:

The resulting spectrum was analyzed using the MNova software package from Mestrelab Research (Santiago de Compostela, Spain). The spectrum was referenced to the internal acetone signal (1H-2.22 ppm; 13C-30.8 ppm) and phased using the Regions2D method in both the F2 and F1 dimension. Apodization using 90 degree shifted sine was applied in both the F2 and F1 dimension. Individual signals (C—H correlations) were quantified by integration of their respective peaks using “predefined integral regions” with elliptical integration shapes. The resulting table of integral regions and values were normalized to a sum of 100 in order for the value to represent a percentage of the total. Peak integral regions were selected to avoid peaks associated with monomers.

Results

Ten batches of the selected oligosaccharide composition produced according to the process of Example 7 (IIIarathon C catalyst) and analyzed by SEC as described in Example 15, were analyzed using the above NMR methods. Collectively, these batches comprised the following NMR peak signals (Table 4).

TABLE 4
HSQC NMR peaks of the selected oligosaccharide composition

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | To |
| 1 | 3.68 | 3.61 | 3.75 | 63.42 | 62.64 | 64.20 |
| 2 | 3.75 | 3.72 | 3.78 | 66.06 | 65.50 | 66.62 |
| 3 | 3.97 | 3.94 | 4.00 | 66.15 | 65.81 | 66.49 |
| 4 | 3.96 | 3.94 | 3.98 | 69.28 | 69.04 | 69.52 |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 8 | 3.97 | 3.94 | 4.00 | 71.56 | 71.29 | 71.84 |
| 9 | 3.72 | 3.67 | 3.77 | 72.35 | 71.95 | 72.74 |
| 15 | 4.44 | 4.41 | 4.46 | 103.86 | 103.56 | 104.15 |
| 10 | 3.33 | 3.27 | 3.4 | 73.74 | 73.26 | 74.22 |
| 14 | 4.5 | 4.47 | 4.54 | 103.29 | 102.87 | 103.70 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |
| 14 | 4.5 | 4.54 | 4.47 | 103.29 | 102.87 | 103.70 |
| 15 | 4.44 | 4.46 | 4.41 | 103.86 | 103.56 | 104.15 |

The relative size of each of the peaks (AUC) collected for the NMR spectra of the selected oligosaccharide composition produced according to the process as described in Example 7 was further determined, as shown below:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

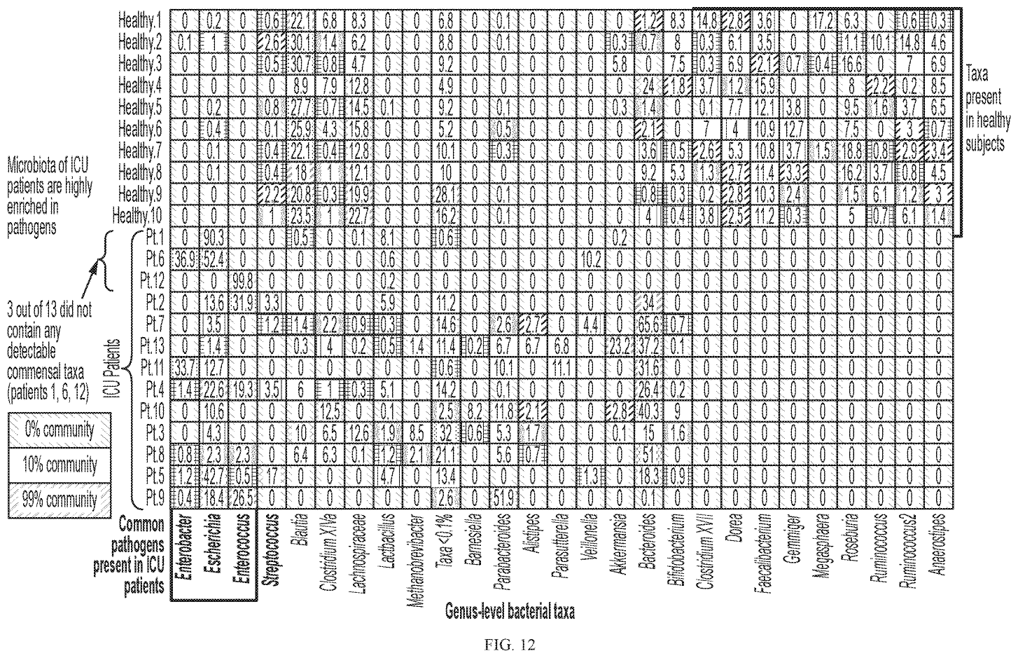

A representative HSQC NMR spectra of the selected oligosaccharide composition is provided in FIG. 11.

Twenty-three batches of the selected oligosaccharide composition produced according to the process of Example 9 (citric acid catalyst) and analyzed by SEC as described in Example 15, were analyzed using the above NMR methods. Collectively, these batches comprised the NMR peak signals as shown in Table 4. The relative size of each of the peaks (AUC) collected for the NMR spectra of the selected oligosaccharide composition produced according to the process as described in Example 9 was further determined, as shown below:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

### Example 15. Size Exclusion Chromatography Method

The weight-average molecular weight (MWw), number-average molecular weight (MWn), and polydispersity index (PDI) of batches and samples of the selected oligosaccharide composition, as produced by the processes in Examples 7 and 9, were determined by SEC HPLC

Method

These methods involved the use of an Agilent 1100 with refractive index (RI) detector equipped with a guard column (Shodex SUGAR SP-G 6B Guard Column 6×50 mm, 10 μm) and two chromatography columns in series: 1) Shodex OHpak SB-802 HQ, 8.0×300 mm, 8 μm, P/N F6429100; 2) Shodex OHpak SB-803 HQ, 8.0×300 mm, 6 μm, P/N F6429102.

The mobile phase (0.1 M NaNO₃) was prepared by weighing 17 g of NaNO₃ (ACS grade reagent) and dissolving in 2000 mL of deionized (DI) water (from MiliQ water filter). The solution was filtered through a 0.2 μm filter.

Polymer standard solutions (10.0 mg/mL) of each of D-(+) Glucose Mp 180, Carbosynth Ltd Standard, or equivalent (CAS #50-99-7); Maltose Mp 342, Carbosynth Ltd Standard, or equivalent (CAS #69-79-4); Maltotetraose Mp 667, Carbosynth Ltd Standard, or equivalent (CAS #34612-38-9); Maltooctaose Mp 1315, Carbosynth Ltd Standard, or equivalent (CAS #6156-84-9); Nominal Mp 6100 Pullulan Standard, PSS #PPS-pul6k; Nominal Mp 9600 Pullulan Standard, PSS #PPS-pul10k; and Nominal Mp 22000 Pullulan Standard, PSS #PPS-pul22k were prepared by weighing 20 mg of a standard into a separate 20 mL scintillation vial and adding 2.0 mL of DI water to each vial. A polymer solution mixture #1 was prepared by weighing 10 mg of each standard of glucose, maltose, maltooctaose and Mp 9600 into an HPLC vial, adding 1.0 mL of diluent and mixing well. A polymer solution mixture #1 was prepared by weighing 10 mg of each standard of maltotetraose, Mp 6100 and Mp 21100 into an HPLC vial, adding 1.0 mL of diluent and mixing well.

Sample A was prepared in duplicate. Approximately 300 mg of oligosaccharide sample was weighed into a 20 mL scintillation vial and 10 mL of DI water was added. The solution was mixed and filtered through a PES syringe filter with a 0.2 μm polyethersulfone membrane.

Sample B was prepared in duplicate. Approximately 220 mg of oligosaccharide sample was weighed into a 20 mL scintillation vial and 10 mL of DI-water was added. The solution was mixed and filtered a PES syringe filter with a 0.2 μm polyethersulfone membrane.

The flow rate was set to 0.7 mL/min at least 2 hours before running samples with the column temperature set to 65° C. and the RI detector temperature set to 50° C. with the RI detector purge turned on.

Before running samples wherein the injection volume for all samples was 10 μL and run time was 40 minutes, the detector purge was turned off and the pump was run at 0.7 mL/min until an acceptable baseline was obtained.

A blank sample consisting of DI water was run. Samples of standard mixtures #1 and #2 were run. Sample A was run. Sample B was run.

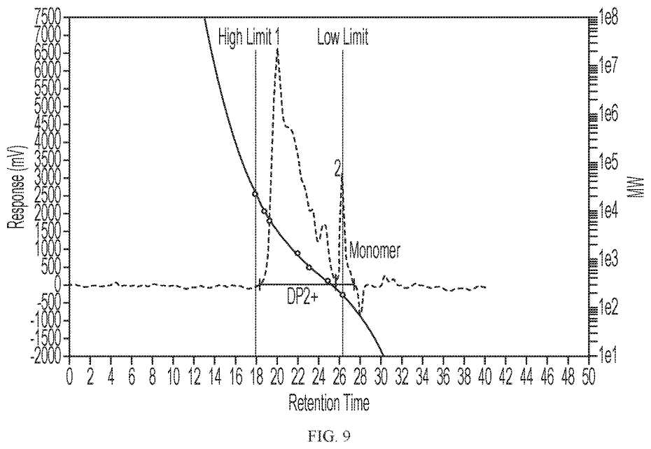

The peaks between 18 and 25.5 minutes were integrated. The monomer and the broad peak (the product) were integrated as shown in the sample chromatogram (FIG. 9). The calibration curve fit type in Empower 3 software was set to 3^{rd} order. The molecular weight distributions and polydispersity were calculated using Empower 3 software for the broad peak. The Mw, Mn and polydispersity of the product peak (DP2+) were determined using these methods.

Results

Fourteen batches of the selected oligosaccharide composition produced using the process in Example 7 (IIIarathon C catalyst) were analyzed using the above SEC methods. The batches of oligosaccharide composition comprised oligosaccharides with an average MWw of 2074 g/mol (ranging from 1905-2286 g/mol), an average MWn of 1097 g/mol (ranging from 1033-1184 g/mol), and an average PDI of 1.9 (ranging from 1.84-1.97). Assayed batches comprised a DP2+ of 91.1% (DP2+ ranging from 86.3-95.9%) and about 8.9% monomer on average (ranging from 4.1-13.8% monomer). Assayed batches had an average degree of polymerization (DP) of 12.7 (ranging from 11.6-14.0).

Twenty-three batches of the selected oligosaccharide composition produced using the process in Example 9 (citric acid catalyst) were analyzed using the above SEC methods. The batches of oligosaccharide composition comprised oligosaccharides with an average MWw of 1998 g/mol (ranging from 1863-2268 g/mol), an average MWn of 1030 g/mol (ranging from 984-1106.00 g/mol), and an average PDI of 1.94 (ranging from 1.88-2.05). Assayed batches comprised a DP2+ of 87.3% (DP2+ ranging from 83.6-91.0%) and about 12.7% monomer on average (ranging from 9.0-16.4% monomer). Assayed batches had an average degree of polymerization (DP) of 12.2 (ranging from 11.4-13.9).

### Example 16. SEC HPLC Methodology for Determination of Impurities

The presence of residual organic acid impurities and related substances of batches and samples of the selected oligosaccharide composition, as produced by the processes in Examples 7 and 9, were determined by SEC HPLC.

Methods

These methods involved the use of an Agilent 1100 with refractive index (RI) detector equipped with a guard column (Bio-Rad MicroGuard Cation H+ Cartridge, PIN 125-0129, or equivalent) and a Bio-Rad Aminex HPX-87H, 300×7.8 mm, 9 μm, PIN 125-0140 column, or equivalent.

The mobile phase (25 mM H₂SO₄ in water) was prepared by filling a bottle with 2000 mL DI-water and slowly adding 2.7 mL of H₂SO₄. The solution was filtered through a 0.2 μm filter.

A standard solution was prepared by measuring 50±2 mg of reference standard into a 100-mL volumetric flask, adding mobile phase to 100-mL mark and mixing well.

A sample of a selected oligosaccharide composition (Sample A) was prepared in duplicate. Approximately 1000 mg of oligosaccharide sample was weighed into a 10 mL volumetric flask and mobile phase was added up to the mark. The solution was mixed and filtered through a PES syringe filter with a 0.2 μm polyethersulfone membrane.

A sample of a selected oligosaccharide composition (Sample B) was prepared in duplicate. Approximately 700 mg of oligosaccharide sample was weighed into a 10 mL volumetric flask and mobile phase was added up to the mark. The solution was mixed and filtered through a PES syringe filter with a 0.2 μm polyethersulfone membrane.

The flow rate was set to 0.65 mL/min at least 2 hours before running samples with the column temperature set to 50° C. and the RI detector temperature set to 50° C. with the RI detector purge turned on.

Before running samples wherein the injection volume for all samples was 50 μL and run time was 40 minutes, the detector purge was turned off and the pump was run at 0.65 mL/min until an acceptable baseline was obtained.

A blank sample consisting of DI water was run. The standard, sample A, and sample B were each independently run.

The peaks at 7.5 min (Glucuronic acid), 9.4 min (Maleic Acid), 11.3 min (Levoglucosan), 11.9 min (Lactic Acid), 13.1 min (Formic Acid), 14.2 min (Acetic Acid), 15.5 min (Levulinic Acid), 31.8 min (hydroxymethylfurfural, HMF), and 8.3 min (Glucose) were integrated. The calibration curve fit type in Empower 3 software was set to 3^{rd} order.

Results

Ten batches of the selected oligosaccharide, as produced by the process in Example 7 (IIIarathon C catalyst), were tested using the method above. The selected oligosaccharide composition comprised 0.35% w/w (±0.05%) levoglucosan, 0.03% w/w (±0.01%) lactic acid, and 0.06% w/w (±0.01%) formic acid. Samples of the selected oligosaccharide composition comprised 0.28-0.43% w/w levoglucosan, 0.00-0.03% w/w lactic acid, and 0.05-0.07% w/w formic acid.

Batches of the selected oligosaccharide, as produced by the process in Example 9 (citric acid catalyst), were tested using the method above. The selected oligosaccharide composition comprised 0.47% w/w (±0.02%) levoglucosan (23 batches of the selected oligosaccharide), 0.01% w/w lactic acid (11 batches of the selected oligosaccharide), 0.02% w/w formic acid (12 batches of the selected oligosaccharide), and 0.02% w/w citric acid (23 batches of the selected oligosaccharide). Samples of the selected oligosaccharide composition comprised 0.43-0.51% w/w levoglucosan, 0.01-0.02% w/w lactic acid, 0.00-0.03% w/w formic acid, and 0.00-0.03% w/w citric acid.

### Example 17. SEC HPLC Methodology for Determination of DP1-DP7

The relative amounts of oligosaccharides with a degree of polymerization (DP) of 1, 2, and 3+ in batches and samples of a selected oligosaccharide composition, as produced by the processes in Examples 7-9 were determined by SEC HPLC.

Methods

These methods involved the use of an Agilent 1100 with refractive index (RI) detector equipped with a guard column (Shodex SUGAR SP-G 6B Guard Column 6×50 mm, 10 μm, P/N F6700081, or equivalent) and a chromatography column (Shodex Sugar SP0810, 8.0×300 mm, 8 μm, P/N F6378105, or equivalent).

The mobile phase (0.1 M NaNO₃) was prepared by weighing 42.5 g of NaNO₃ (ACS grade reagent) and dissolving in 5000 mL of deionized (DI) water (from MiliQ water filter). The solution was filtered through a 0.2 μm filter.

Polymer standard solutions (10.0 mg/mL) of each of D-(+) Glucose Mp 180, Carbosynth Ltd Standard, or equivalent (CAS #50-99-7) (DPI); Maltose Mp 342, Carbosynth Ltd Standard, or equivalent (CAS #69-79-4) (DP2); Maltotriose Mp 504, Carbosynth Ltd Standard, or equivalent (CAS #1109-28-0) (DP3); Maltotetraose Mp 667, Carbosynth Ltd Standard, or equivalent (CAS #34612-38-9) (DP4); Maltopentaose Mp 828, Carbosynth Ltd Standard, or equivalent (CAS #34620-76-3) (DP5); Maltohexaose Mp 990, Carbosynth Ltd Standard, or equivalent (CAS #34620-77-4) (DP6); Maltoheptaose Mp 1153, Carbosynth Ltd Standard, or equivalent (CAS #34620-78-5) (DP7); and Maltooctaose Mp 1315, Carbosynth Ltd Standard, or equivalent (CAS #6156-84-9) (DP8), were prepared by weighing 10 mg of a standard into an individual 1.5 mL centrifuge tube and adding DI water to make 10 mg/mL solution.

Samples of the selected oligosaccharide composition were prepared as 10mg/mL concentrated samples or dilute aqueous samples to 2.5-3.5 Brix.

The flow rate was set to 1.0 mL/min at least 2 hours before running samples with the column temperature set to 70° C. and the RI detector temperature set to 40° C. with the RI detector purge turned on.

Before running samples wherein the injection volume for all samples was 5 μL and run time was 15 minutes, the detector purge was turned off and the pump was run at 1.0 mL/min until an acceptable baseline was obtained.

A blank sample consisting of DI water, individual standards, and sample were independently run.

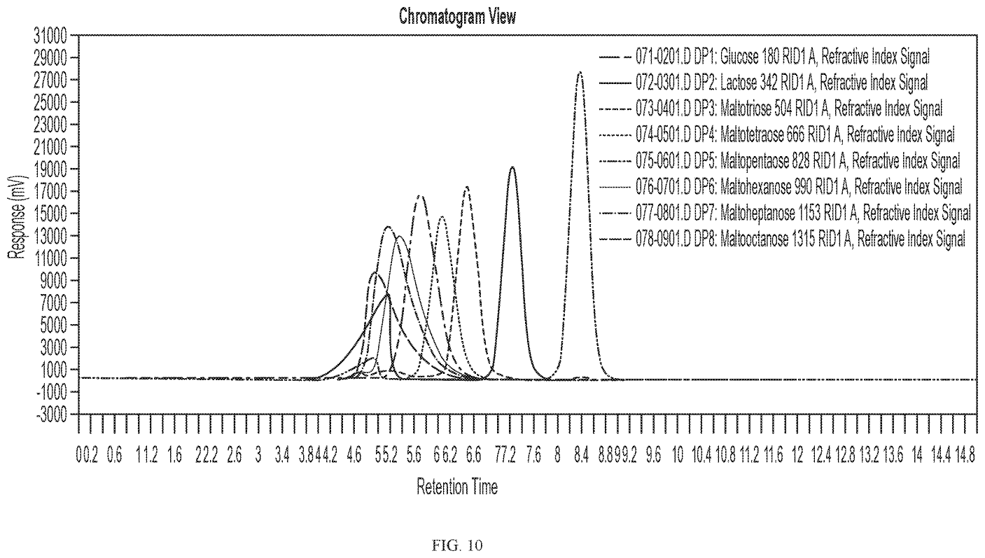

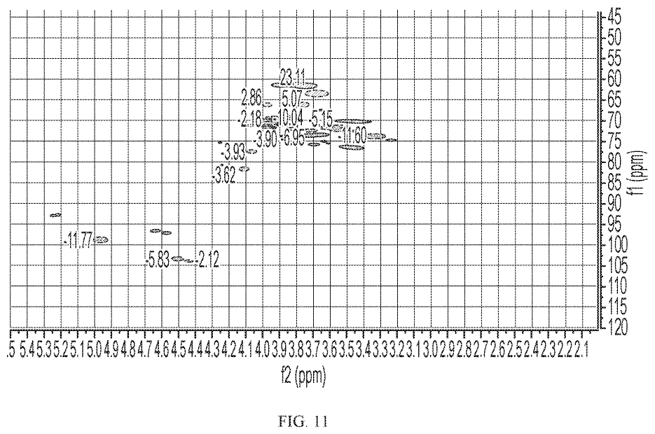

Each peak between 4 and 9.2 minutes in the sample run, corresponding to individual standards, was integrated. An overlay of the standards in shown in FIG. 10. The calibration curve fit type in Empower 3 software was set to 3^{rd} order. The DP1, DP2, and DP3+ values of the samples (samples of selected oligosaccharide composition) were determined using these methods.

Results

Ten samples of selected oligosaccharide composition produced using the process in Example 7 (IIIarathon C catalyst) were assayed using this method. The selected oligosaccharide composition comprised 5.24%(±0.35%) monomer (DPI), 7.52%(±0.44%) disaccharide (DP2), and 87.25%(±0.78%) oligomers having at least three linked monomer units (DP3+).

### Example 18. Determination of Total Dietary Fiber

The amount of dietary fiber in batches of the selected oligosaccharide composition, as produced by the process in Example 7 (IIIarathon C catalyst) were measured according to the methods of AOAC 2011.25 (AOAC International, AOAC Official Method 2011.25). The average amount of total dietary fiber was 87.44% (on dry basis) across 10 batches (ranging from 84.9-90.5%). The percent Dextrose Equivalent (DE) (dry basis) of these oligosaccharide batches was also measured according to the Food Chemicals Codex (FCC). The average amount of dextrose equivalent (on dry basis) was 16.60% across two batches (one at 15.10% DE and the other at 18.10% DE).

The amount of dietary fiber and dextrose equivalents (DE) in batches of the selected oligosaccharide composition, as produced by the process in Example 9 (citric acid catalyst) were measured according to the methods of AOAC 2011.25 (AOAC International, AOAC Official Method 2011.25). The percent Dextrose Equivalent (DE) (dry basis) of these oligosaccharide batches was also measured according to the Food Chemicals Codex (FCC). The average amount of total dietary fiber was 64.14% (on dry basis) across fourteen batches (ranging from 47.10-73.10%). The average amount of dextrose equivalent (on dry basis) was 20.60% across two batches (one at 18.60% DE and the other at 22.60% DE).

### Example 19. Clinical Trial to Assess Ability of the Selected Oligosaccharide Composition to Reduce the Relative or Absolute Abundance of Pathogens

The ability of the selected oligosaccharide composition comprised of a plurality of oligosaccharides selected from Formula (I), Formula (II), and Formula (III) as produced by a similar process as described in Examples 7-9 is assessed for its ability to reduce the abundance (e.g., relative abundance or absolute abundance) of pathogenic bacteria (e.g., Enterobacteriaceae, Enterococcus, and C. difficile).

The study is a randomized, controlled, multi-site, open label clinical study to assess the selected oligosaccharide composition on safety as well as the proportion of subjects with a clinically significant reduction from baseline in abundance of taxa of interest (Enterobacteriaceae, Enterococcus, and C. difficile, combined) as measured by metagenomic sequencing.

A reasonable number of patients are randomized 3:2 to a treatment group (i.e., treatment with the selected oligosaccharide composition) or to an observational control group.

Patients are at least 18 years of age and must have a positive fecal sample for VRE, ESBLE, or CRE, based on a 16S metagenomic analysis of a fecal sample of the patient.

Patients in the treatment group are administered (e.g., orally self-administered) a dosage of the oligosaccharide composition (e.g., once or twice daily) for the length of the study (e.g., 28 days). Patients in the treatment group and the observational group undergo regular physical checkups throughout the length of the study, which may include observation of vital signs, fecal stool sample collection for microbiologic and/or 16S metagenomic testing, recordation of stool frequency and BSS evaluation, and/or recordation of adverse effects.

The endpoints of the study may include the number of patients experiencing study product-related treatment emergent adverse events (TEAEs); serious adverse events (SAEs), change from baseline in vital signs, electrocardiograms (ECGs), physical examinations, safety laboratory analyses; and change from baseline in stool frequency, Bristol Stool Score (BSS); and the proportion of subjects with a reduction from baseline (e.g., a ≥30% reduction) at the end of the study (e.g., on Day 28) in the abundance (e.g., relative abundance or absolute abundance) of taxa of interest (Enterobacteriaceae, Enterococcus, and C. difficile, combined and/or individually) as measured by metagenomic sequencing.

Additional endpoints may include (i) proportion of subjects with a reduction in level of MDR organisms (cfu/g feces by culture) including VRE, ESBLE, and CRE, combined and/or individually; (ii) alpha diversity (Shannon index) by nucleic acid sequencing over the intake phase versus baseline; (iii) abundance of bacteria as measured by nucleic acid sequencing over the intake phase versus baseline; (iv) change in level of stool inflammatory biomarkers (e.g., lipocalin) over the intake phase versus baseline; (v) Change in blood inflammatory markers (e.g., IFN-γ, IL-1β, IL-2, IL-4, IL-6, IL-8, IL-10, IL-12p70, IL-13, and TNF-α) over the intake phase versus baseline; (vi) change in microbial metabolite concentration (for example, p-cresol sulfate, trimethylamine oxide) in stool, urine or blood samples over the intake phase versus baseline; (vii) change in abundance (e.g., relative abundance or absolute abundance) of bacterial taxa as determined by nucleic acid sequencing of stool samples at baseline versus over the intake phase, compared with changes in bacterial taxa seen in an ex vivo culture system using subject stool samples; and/or (viii) rate of infections and hospitalizations per subject per week over intake and washout phases in treatment group versus control control group.

### Equivalents and Terminology

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A method of reducing the relative or absolute abundance of pathogens in the gastrointestinal tract of a human subject, the method comprising administering to the gastrointestinal tract of the subject an effective amount of an oligosaccharide composition, wherein the oligosaccharide composition comprises a plurality of oligosaccharides selected from Formula (I), Formula (II), and Formula (III): 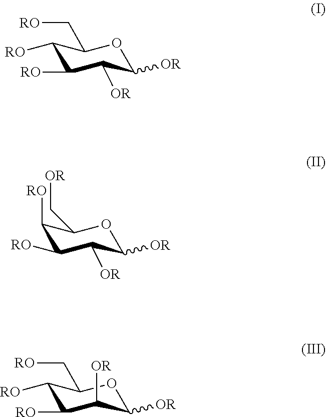
wherein each R independently is selected from hydrogen, and Formulae (Ia), (Ib), (Ic), (Id), (IIa), (IIb), (IIc), (IId), (IIIa), (IIIb), (IIIc), (IIId): 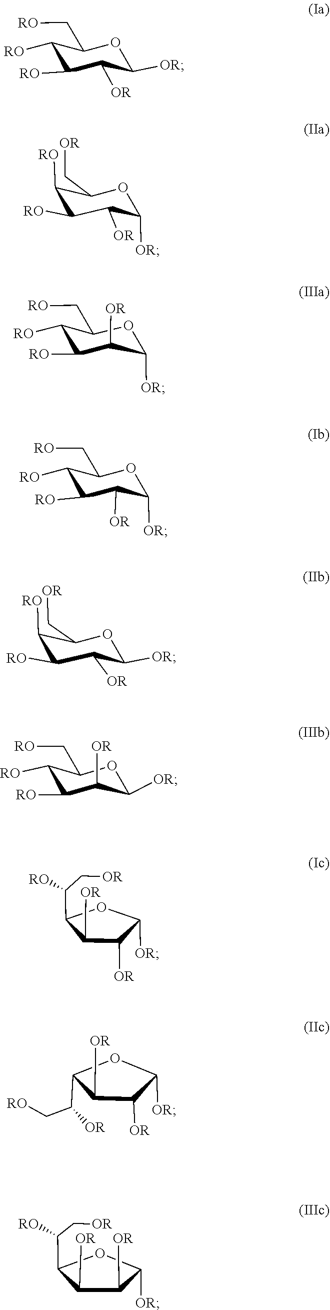 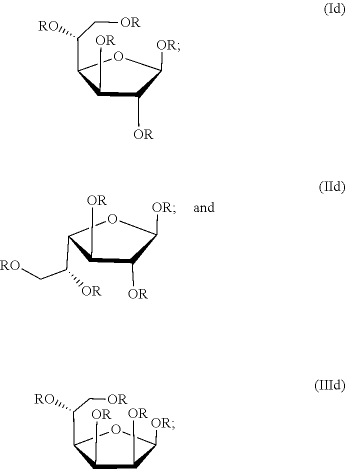
wherein each R independently is as defined above;
wherein the oligosaccharide composition is produced by a process comprising:
(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and
(b) promoting acid catalyzed oligosaccharide formation in the reaction mixture by transferring sufficient heat to the reaction mixture to maintain the reaction mixture at its boiling point.

2. The method of claim 1, wherein step (b) comprises promoting acid catalyzed oligosaccharide formation in the reaction mixture by transferring sufficient heat to the reaction mixture to maintain the reaction mixture at its boiling point until the weight percent of total monomer content in the oligosaccharide composition is in a range of 2% to 20%, wherein the total monomer content comprises dextrose monomer, galactose monomer, and/or mannose monomer.

3. The method of claim 1 or 2, wherein the mean degree of polymerization of all oligosaccharide compositions is in a range of 7-15.5.

4. The method of any one of claims 1-3, wherein the method reduces the abundance of pathogenic bacteria in the gastrointestinal tract.

5. The method of any one of claims 1-4, wherein the method increases the abundance of commensal bacteria in the gastrointestinal tract.

6. The method of any one of claims 1-5, wherein the relative or absolute abundance of pathogens is determined by performing nucleic acid sequencing (e.g., 16S metagenomic sequencing) of a sample collected from the subject (e.g., a fecal sample).

7. The method of claim 6, wherein the reduction of the relative or absolute abundance of pathogens is determined by:
(i) performing nucleic acid sequencing (e.g., 16S metagenomic sequencing) of a sample collected from the subject prior to administration of the oligosaccharide composition or obtaining such data;
(ii) performing nucleic acid sequencing (e.g., 16S metagenomic sequencing) of a sample collected from the subject following administration of the oligosaccharide composition or obtaining such data; and
(iii) comparing the relative or absolute abundance of pathogens using the sequencing data provided in (ii) relative to the relative or absolute abundance of pathogens using the sequencing data provided in (i).

8. An oligosaccharide composition comprising a plurality of oligosaccharides selected from Formula (I), Formula (II), and Formula (III): 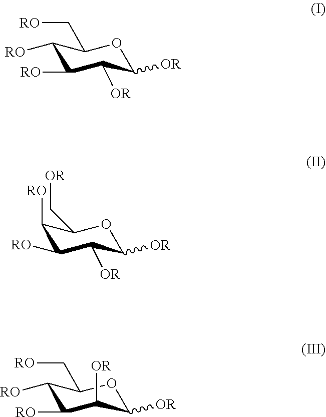
wherein each R independently is selected from hydrogen, and Formulae (Ia), (Ib), (Ic), (Id), (IIa), (IIb), (IIc), (IId), (IIIa), (IIIb), (IIIc), (IIId): 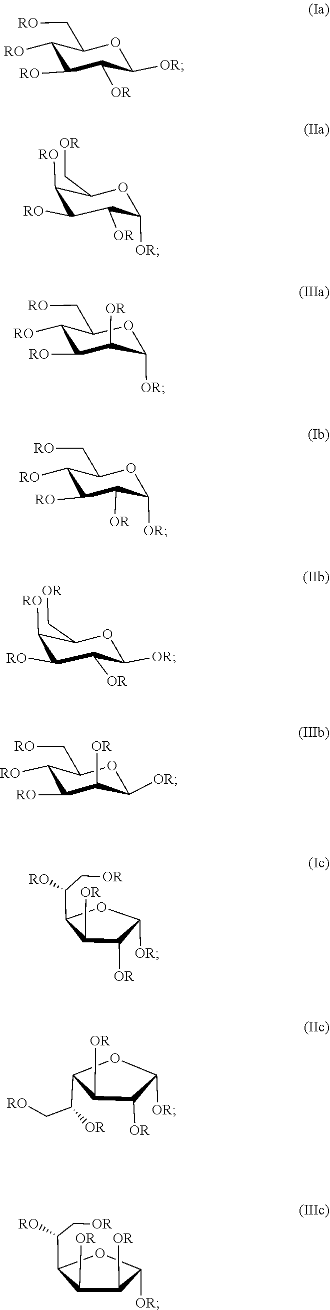 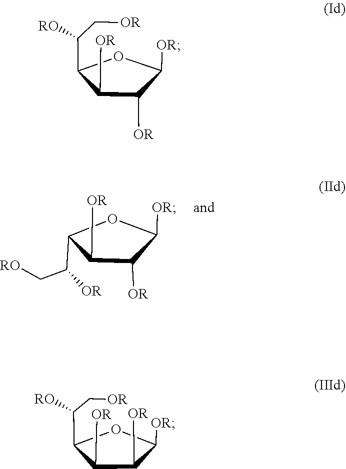
wherein each R independently is as defined above;
wherein the oligosaccharide composition is produced by a process comprising:
(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and
(b) promoting acid catalyzed oligosaccharide formation in the reaction mixture by transferring sufficient heat to the reaction mixture to maintain the reaction mixture at its boiling point.

9. The composition of claim 8, wherein step (b) comprises promoting acid catalyzed oligosaccharide formation in the reaction mixture by transferring sufficient heat to the reaction mixture to maintain the reaction mixture at its boiling point until the weight percent of total monomer content in the oligosaccharide composition is in a range of 2% to 20%, wherein the total monomer content comprises dextrose monomer, galactose monomer, and/or mannose monomer.

10. An oligosaccharide composition comprising a plurality of oligosaccharides selected from Formula (I), Formula (II), and Formula (III): 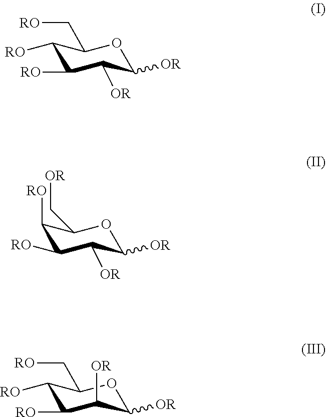
wherein each R independently is selected from hydrogen, and Formulae (IIa), (IIb), (IIc), (IId), (IIIa), (IIIb), (IIIc), (IIId): 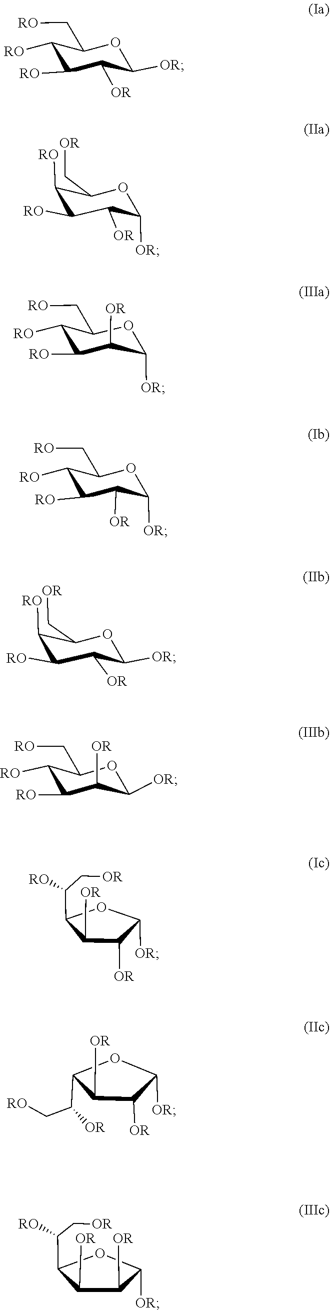 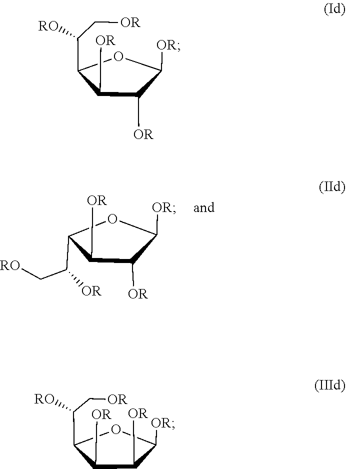
wherein each R independently is as defined above;
wherein the oligosaccharide composition is produced by a process comprising:
(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and
(b) maintaining the reaction mixture at its boiling point, at a pressure in the range of 0.5-1.5 atm, under conditions that promote acid catalyzed oligosaccharide formation, until the weight percent of total monomer content in the oligosaccharide composition is in a range of 2% to 20%, wherein the total monomer content comprises dextrose monomer, galactose monomer, and/or mannose monomer.

11. An oligosaccharide composition comprising a plurality of oligosaccharides that are minimally digestible in humans, the composition being characterized by a multiplicity-edited gradient-enhanced ¹H-¹³C heteronuclear single quantum correlation (HSQC) NMR spectrum comprising signals 5, 6, 7, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

12. The composition of claim 11, wherein the oligosaccharide composition is characterized by a multiplicity-edited gradient-enhanced ¹H-¹³C heteronuclear single quantum correlation (HSQC) NMR spectrum comprising signals 5, 6, 7, 10, 14, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

13. The composition of claim 11 or 12, wherein the oligosaccharide composition is characterized by a multiplicity-edited gradient-enhanced ¹H-¹³C heteronuclear single quantum correlation (HSQC) NMR spectrum comprising signals 5, 6, 7, and 10-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

14. The composition of any one of claims 11-13, wherein the oligosaccharide composition is characterized by a multiplicity-edited gradient-enhanced ¹H-¹³C heteronuclear single quantum correlation (HSQC) NMR spectrum comprising signals 1-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

15. The composition of any one of claims 11-14, wherein any one of signals 1-15 are further characterized by an ¹H integral region and a ¹³C integral region, defined as follows:

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | to |
| 1 | 3.68 | 3.61 | 3.75 | 63.42 | 62.64 | 64.20 |
| 2 | 3.75 | 3.72 | 3.78 | 66.06 | 65.50 | 66.62 |
| 3 | 3.97 | 3.94 | 4.00 | 66.15 | 65.81 | 66.49 |
| 4 | 3.96 | 3.94 | 3.98 | 69.28 | 69.04 | 69.52 |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 8 | 3.97 | 3.94 | 4.00 | 71.56 | 71.29 | 71.84 |
| 9 | 3.72 | 3.67 | 3.77 | 72.35 | 71.95 | 72.74 |
| 10 | 3.33 | 3.27 | 3.4 | 73.74 | 73.26 | 74.22 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |
| 14 | 4.5 | 4.47 | 4.54 | 103.29 | 102.87 | 103.70 |
| 15 | 4.44 | 4.41 | 4.46 | 103.86 | 103.56 | 104.15 |

16. The composition of any one of claims 11-15, wherein the NMR spectrum is obtained by subjecting a sample of the composition to a multiplicity-edited gradient-enhanced ¹H-¹³C heteronuclear single quantum coherence (HSQC) experiment using an echo-antiecho scheme for coherence selection using the following pulse sequence diagram, acquisition parameters and processing parameters:

[symbol]

17. The composition of any one of claims 11-16, wherein the NMR spectrum is obtained by subjecting a sample of the oligosaccharide composition to HSQC NMR, wherein the sample is dissolved in D2O.

18. The composition of any one of claims 11-17, wherein the oligosaccharide composition has been subjected to a de-monomerization procedure.

19. The composition of any one of claims 11-17, wherein the composition comprises a plurality of oligosaccharides selected from Formula (I), Formula (II), and Formula (III): 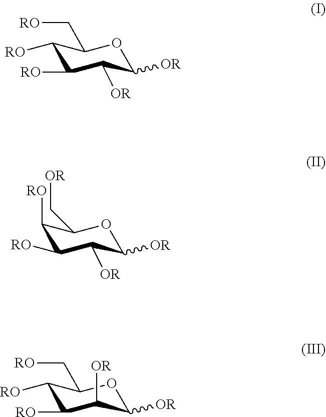
wherein each R independently is selected from hydrogen, and Formulae (Ia), (Ib), (Ic), (Id), (IIa), (IIb), (IIc), (IId), (IIIa), (IIIb), (IIIc), (IIId): 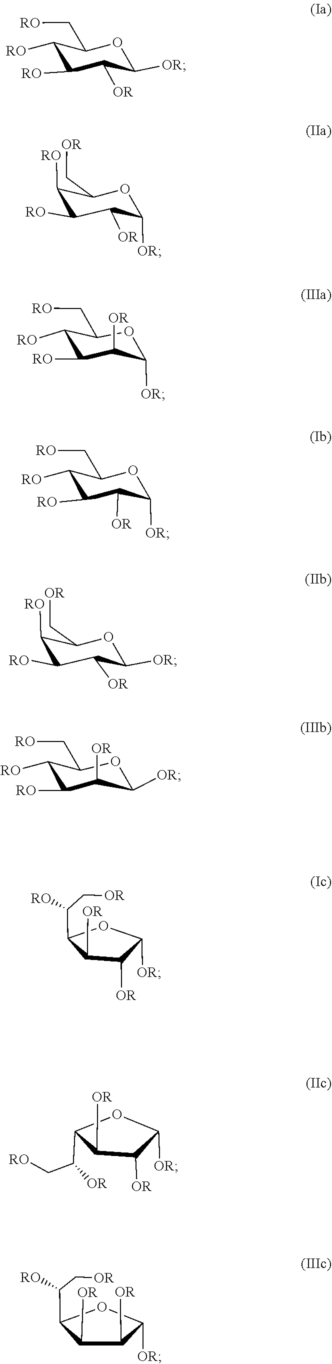 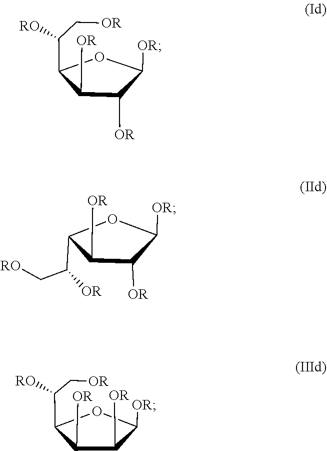
wherein each R independently is as defined above;
wherein the oligosaccharide composition is produced by a process comprising:
(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and
(b) promoting acid catalyzed oligosaccharide formation in the reaction mixture by transferring sufficient heat to the reaction mixture to maintain the reaction mixture at its boiling point.

20. The composition of any one of claim 8-10 or 19, wherein step (b) comprises loading the reaction mixture with an acid catalyst comprising positively charged hydrogen ions, in an amount such that the molar ratio of positively charged hydrogen ions to total dextrose monomer, galactose monomer, and mannose monomer content is in an appropriate range.

21. The composition of any one of claim 8-10 or 19-20, wherein steps (a) and (b) occur simultaneously.

22. The composition of any one of claim 8-10 or 19-21, wherein step (a) comprises heating the reaction mixture under agitation conditions to a temperature in a range of 100° C. to 160° C.

23. The composition of claim 22, wherein step (a) comprises heating the reaction mixture under agitation conditions to a temperature in a range of 135° C. to 145° C.

24. The composition of any one of claim 8-10 or 19-23, wherein step (a) comprises heating the reaction mixture under agitation conditions at a temperature in a range of 100° C. to 160° C.

25. The composition of claim 24, wherein step (a) comprises heating the reaction mixture under agitation conditions at a temperature in a range of 135° C. to 145° C.

26. The composition of any one of claim 8-10 or 19-25, wherein step (a) comprises gradually increasing the temperature (e.g., from room temperature) to about 140° C., under suitable conditions to achieve homogeneity and uniform heat transfer.

27. The composition of any one of claim 8-10 or 19-26, wherein step (b) comprises maintaining the reaction mixture at atmospheric pressure or under vacuum, at a temperature in a range of 135° C. to 145° C., under conditions that promote acid catalyzed oligosaccharide composition formation, until the weight percent of dextrose monomer, galactose monomer, and mannose monomer in the oligosaccharide composition is in a range of 4-14.

28. The composition of any one of claim 8-10 or 19-27, wherein step (b) comprises gradually increasing the temperature (e.g., from room temperature) to about 140° C., under suitable conditions to achieve homogeneity and uniform heat transfer.

29. The composition of any one of claim 8-10 or 19-28, wherein the acid catalyst is a strong acid cation exchange resin having one or more physical and chemical properties according to Table 1 and/or wherein the catalyst comprises >3.0 mmol/g sulfonic acid moieties and <1.0 mmol/gram cationic moieties.

30. The composition of claim 29, wherein the catalyst has a nominal moisture content of 45-50 weight percent.

31. The composition of any one of claim 8-10 or 19-30, wherein the acid catalyst is a soluble catalyst.

32. The composition of claim 31, wherein the soluble catalyst is an organic acid,

33. The composition of claim 30 or 32, wherein the soluble catalyst is a weak organic acid.

34. The composition of any one of claims 31-33, wherein the soluble catalyst is citric acid.

35. The composition of any one of claim 8-10 or 19-34 wherein the process further comprises:
(c) quenching the reaction mixture, for example, using water, while bringing the temperature of the reaction mixture to a temperature in the range of 55° C. to 95° C. (e.g., 85° C., 90° C.).

36. The composition of claim 35 wherein the process further comprises:
(d) separating oligosaccharide composition from the acid catalyst.

37. The composition of claim 36, wherein in (d) said separating comprises removing the catalyst by filtration.

38. The composition of claim 36 or 37, wherein (d) comprises cooling the reaction mixture to below about 85° C. before filtering.

39. The composition of any one of claims 36-38, wherein the process further comprises:
(e) diluting the oligosaccharide composition of (d) with water to a concentration of about 45-55 weight percent;
(f) passing the diluted composition through a cationic exchange resin;
(g) passing the diluted composition through a decolorizing polymer resin; and/or
(h) passing the diluted composition through an anionic exchange resin;
wherein each of (f), (g), and (h) can be performed one or more times in any order.

40. The composition of any one of claim 8-10 or 19-39, wherein the mean degree of polymerization of all oligosaccharide compositions is in a range of 7-15.5.

41. The composition of any one of claim 8-10 or 19-40, wherein the mean degree of polymerization of all oligosaccharide compositions is in a range of 11-15.

42. The composition of any one of claim 8-10 or 19-41, wherein said heating comprises melting the reaction mixture and/or heating the reaction mixture under suitable conditions to achieve homogeneity and uniform heat transfer.

43. The composition of any one of claim 8-10 or 19-42, wherein said heating comprises melting the reaction mixture and/or heating the reaction mixture under suitable conditions to achieve homogeneity and uniform heat transfer.

44. The composition of any one of claim 8-10 or 19-43, wherein (b) further comprises removing water from the reaction mixture by evaporation.

45. The composition of any one of claim 8-10 or 19-44, wherein (b) further comprises maintaining the reaction mixture at 93-94 weight percent dissolved solids.

46. The composition of any one of claims 35-45, wherein in (c) the water is deionized water.

47. The composition of any one of claims 35-46, wherein in (c) the water has a temperature of about 95° C.

48. The composition of any one of claims 37-47, wherein in (c) the water is added to the reaction mixture under conditions sufficient to avoid solidifying the mixture.

49. The composition of any one of claims 36-48, wherein in (d) said separating comprises removing the catalyst by filtration.

50. The composition of any one of claims 36-49, wherein (d) comprises cooling the reaction mixture to below about 85° C. before filtering.

51. The composition of any one of claims 36-50, wherein the process further comprises diluting the oligosaccharide composition of (d) with water to a concentration of about 35-55 weight percent and passing the diluted composition through a 45 μm filter.

52. The composition of any one of claim 8-10 or 19-51, further comprising water at a level below that which is necessary for microbial growth upon storage at room temperature.

53. The composition of any one of claim 8-10 or 19-52, wherein the composition comprises water in a range of 45-55 weight percent.

54. The composition of any one of claims 8-53, having a MWw (g/mol) in a range of 1905-2290.

55. The composition of any one of claims 8-53, having a MWw (g/mol) in a range of 1740-2407.

56. The composition of any one of claims 8-53, having a MWw (g/mol) in a range of 1863-2268.

57. The composition of any one of claims 8-53, having a MWw (g/mol) in a range of 1700-2295.

58. The composition of any one of claims 8-57, having a MWn (g/mol) in a range of 1033-1184.

59. The composition of any one of claims 8-57, having a MWn (g/mol) in a range of 975-1155.

60. The composition of any one of claims 8-57, having a MWn (g/mol) in a range of 984-1106.

61. The composition of any one of claims 8-57, having a MWn (g/mol) in a range of 938-1120.

62. The composition of any one of claims 8-61, wherein a solution comprising the oligosaccharide composition has a pH in a range of 2.50-7.00,

63. The composition of any one of claims 8-62, wherein a solution comprising the oligosaccharide composition has a pH in a range of 2.50-3.50.

64. The composition of any one of claims 8-63, wherein the composition comprises oligomers having two or more repeat units (DP2+) in a range of 86-96 weight percent.

65. The composition of any one of claims 8-63, wherein the composition comprises oligomers having two or more repeat units (DP2+) in a range of 81-100 weight percent.

66. The composition of any one of claims 8-65, wherein the composition comprises oligomers having at least three linked monomer units (DP3+) in a range of 85-90 weight percent.

67. The composition of any one of claims 8-66, wherein the composition further comprises:

0. 18-0.51% w/w levoglucosan, 0.01-0.05% w/w lactic acid, and/or 0.04-0.07% w/w formic acid.

68. The composition of any one of claims 8-67, wherein the composition further comprises:

0. 40-0.53% w/w levoglucosan, 0.01-0.02% w/w lactic acid, 0.01-0.04% w/w formic acid, and/or 0.01-0.04% w/w citric acid.

69. The composition of any one of claims 8-68, wherein the composition is substantially non-absorbable in a human.

70. A method of reducing a ratio of pathogenic bacteria to commensal bacteria in the gastrointestinal tract of a human subject, the method comprising administering to the gastrointestinal tract of the subject an effective amount of an oligosaccharide composition according to any one of claims 8-69.

71. A method of reducing the relative or absolute abundance of pathogens in the gastrointestinal tract of a human subject, the method comprising administering to the gastrointestinal tract of the subject an effective amount of an oligosaccharide composition according to any one of claims 8-69.

72. The method of claim 70 or 71, wherein the oligosaccharide composition is administered in an amount effective to reduce or inhibit colonization or to increase decolonization of the pathogen in the gut (e.g., small intestine, large intestine and/or colon) of the human subject.

73. The method of claim 70 or 71, wherein the method reduces the abundance of pathogenic bacteria in the gastrointestinal tract, relative to a control (e.g., a control subject or baseline measurement).

74. The method of any one of claims 70-73, wherein the method increases the abundance of commensal bacteria in the gastrointestinal tract, relative to a control (e.g., a control subject or baseline measurement).

75. The method of any one of claims 70-74, wherein the reduction of the relative or absolute abundance of pathogens is determined by performing nucleic acid sequencing (e.g., 16S metagenomic sequencing) of a sample collected from the subject (e.g., a fecal sample).

76. The method of claim 75, wherein the reduction of the relative or absolute abundance of pathogens is determined by:
(i) performing nucleic acid sequencing (e.g., 16S metagenomic sequencing) of a sample collected from the subject prior to administration of the oligosaccharide composition or obtaining such data;
(ii) performing nucleic acid sequencing (e.g., 16S metagenomic sequencing) of a sample collected from the subject following administration of the oligosaccharide composition or obtaining such data; and
(iii) comparing the relative or absolute abundance of pathogens using the sequencing data provided in (ii) relative to the relative or absolute abundance of pathogens using the sequencing data provided in (i).

77. A method of treating a subject for a pathogen infection, the method comprising administering to the gastrointestinal tract of the subject an effective amount of an oligosaccharide composition according to any one of claims 8-69, thereby treating the subject.

78. A method of treating a subject for a pathogen infection, the method comprising administering to the gastrointestinal tract of the subject an effective amount of an oligosaccharide composition, wherein the oligosaccharide composition has an average degree of polymerization of 5-20 and comprises a plurality of oligosaccharides selected from Formula (I), Formula (II), and Formula (III): 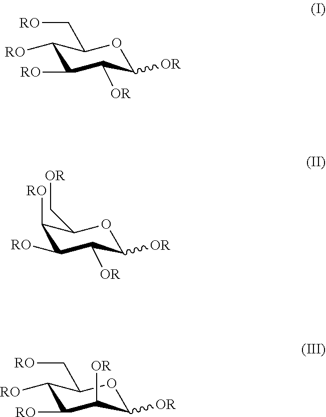
wherein each R independently is selected from hydrogen, and Formulae (Ia), (Ib), (Ic), (Id), (IIa), (IIb), (IIc), (IId), (IIIa), (IIIb), (IIIc), (IIId): 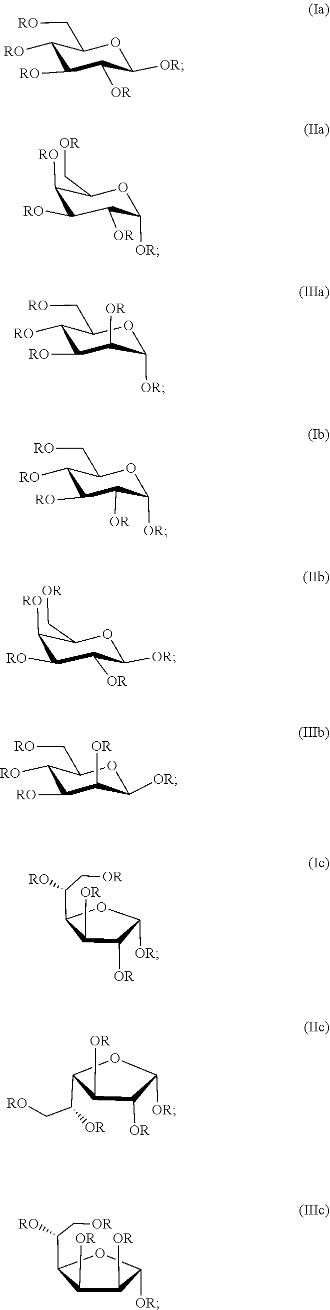 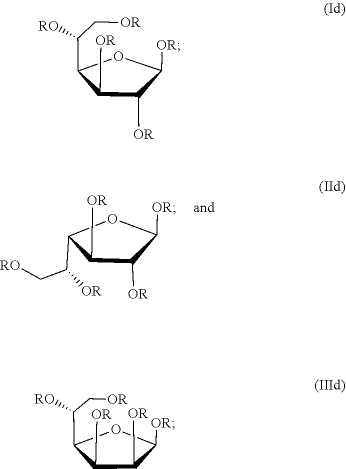
wherein each R independently is as defined above;
thereby treating the subject.

79. The method of claim 77 or 78, wherein the method reduces the rate of infection.

80. The method of any one of claims 77-79, wherein the method reduces the abundance of pathogen.

81. The method of 80, wherein the method reduces the abundance of pathogen of infection by at least 5%, 10%, 20%, or 30%, relative to a baseline measurement (e.g., wherein the baseline measurement is determined prior to treatment).

82. The method of any one of claims 77-81, wherein the method reduces the colonization of the gastrointestional tract by the pathogen.

83. The method of any one of claims 77-82, wherein the method prevents the onset of an infection.

84. The method of any one of claims 77-83, wherein the pathogen infection is an infection of the gastrointestinal tract, lungs, bloodstream, central nervous system, lymphatic system, and/or soft tissues of the subject.

85. The method of any one of claims 70-84, wherein the oligosaccharide composition is administered in an amount sufficient, to reduce or prevent dysbiosis in the gut (e.g., small intestine, large intestine and/or colon) of the human subject.

86. The method of any one of claims 70-85, wherein the oligosaccharide composition reduces the risk of an adverse effect of the pathogen on the human subject.

87. The method of any one of claims 70-86, wherein the oligosaccharide composition is administered in an amount effective to:
a) reduce pathogen biomass (e.g., the number of pathogens and/or the number of drug- or antibiotic-resistance gene or MDR element carriers);
b) modulate (e.g., increase) the level of anti-microbial compounds produced by the subject (e.g., by the resident gut microbiota and/or the host (e.g., human cells));
c) modulate the environment of the GI tract (e.g., small intestine, large intestine or colon), e.g. reducing the pH (e.g., by increasing production or levels of lactic acid, e.g. produced by the resident gut microbiota);
d) modulate (e.g., reduce) a conjugation property of a donor microbe of a drug- or antibiotic-resistance gene or MDR element or modulate (e.g., reduce) the ability of a donor microbe to share a drug- or antibiotic-resistance gene or MDR element with a recipient;
e) reduce the number of drug- or antibiotic-resistance gene or MDR element recipients;
f) reduce the copy number of a drug- or antibiotic-resistance gene or MDR element (e.g. total copy number, e.g. in a donor microbe); and/or
g) increase the fitness cost of the maintenance of antibiotic resistance genes or elements, in the human subject.

88. The method of any one of claims 70-87, wherein the oligosaccharide composition is administered in an amount effective to:
a) decrease the relative or absolute abundance of pathogens and/or drug- or antibiotic-resistance gene or MDR element carriers; and
b) increase the relative or absolute abundance of commensal or beneficial bacteria.

89. The method of any of claims 70-88, wherein the pathogen is a bacterial microorganism (e.g., non-antibiotic resistant) or a fungal microorganism.

90. The method of any of claims 70-89, wherein the pathogen is a drug or antibiotic resistant pathogen, optionally a multi-drug resistant (MDR) pathogen.

91. The method of any of claims 70-90, wherein the pathogen is vancomycin resistant Enterococcus (VRE) or carbapenem resistant Enterobacteriaceae (CRE).

92. The method of any of claims 70-90, wherein the pathogen is VRE Enterococcus faecium.

93. The method of any of claims 70-90, wherein the pathogen is CRE Escherichia coli or CRE Klebsiella pneumoniae.

94. The method of any of claims 70-90, wherein the pathogen is Candida albicans, Candida glabrata, Candida krusei, Candida tropicalis, or Candida lusitaniae.

95. The method of any of claims 70-90, wherein the pathogen is Clostridium difficile.

96. The method of any of claims 70-90, wherein the pathogen is gram-positive bacteria or gram-negative bacteria.

97. The method of any of claims 70-90, wherein the pathogen is a fungus.

98. The method of claim 97, wherein the pathogen is Candida.

99. The method of any of claims 70-98, wherein the human subject:
(i) has received cancer treatment;
(ii) is a transplant recipient;
(iii) has received immunosuppression;
(iv) has an auto-immune disease (e.g., systemic lupus erythematosus, rheumatoid arthritis, Sjögren's syndrome, or Crohn's disease);
(v) has a hematological malignancy;
(vi) has cirrhosis (e.g., including end-stage liver disease (ESLD))
(vii) is preparing for or recovering from a gastrointestinal surgery
(viii) is a patient in an intensive care unit (ICU);
(ix) has had multiple courses of antibiotics, and/or chronic use of antibiotics;
(x) has a positive stool culture for Carbapenem-resistant Enterobacteriaciae (CRE), extended spectrum beta lactamase (ESBL) producing Enterobacteriaciae (ESBLE), and/or Vancomycin-resistant Enterococcus (VRE);
(xi) has low diversity of bacterial communities in the gastrointestinal tract; and/or
(xii) has recently had a central line-associated bloodstream infection (CLABSI), a catheter-associated urinary tract infection (CAUTI), or a C. difficile infections).

100. The method of claim 99, wherein the transplant recipient is a hematopoietic stem cell transplant (HSCT) recipient and/or a solid organ transplant recipient.

101. A method comprising:
(a) identifying a human subject who
(i) has received cancer treatment; (ii) is a transplant recipient; (iii) has received immunosuppression; (iv) has an auto-immune disease (e.g., systemic lupus erythematosus, rheumatoid arthritis, Sjögren's syndrome, or Crohn's disease); (v) has a hematological malignancy; (vi) has cirrhosis (e.g., including end-stage liver disease (ESLD)); (vii) is preparing for or recovering from a gastrointestinal surgery; (viii) is a patient in an intensive care unit (ICU); (ix) has had multiple courses of antibiotics, and/or chronic use of antibiotics; (x) has a positive stool culture for Carbapenem-resistant Enterobacteriaciae (CRE), extended spectrum beta lactamase (ESBL) producing Enterobacteriaciae (ESBLE), and/or Vancomycin-resistant Enterococcus (VRE); (xi) has low diversity of bacterial communities in the gastrointestinal tract; (xii) has increased levels of a drug or antibiotic resistant pathogen (e.g., VRE, CRE, Candida, or Clostridium difficile), gram-positive bacteria or gram-negative bacteria; and/or (xiii) has recently had a central line-associated bloodstream infection (CLABSI), a catheter-associated urinary tract infection (CAUTI), or a C. difficile infections); and
(b) treating the subject with an effective amount of an oligosaccharide composition according to any one of claims 8-69.

102. The method of any one of claims 70-101 further comprising administering to the human subject a population of commensal or probiotic bacteria.

103. The method of claim 102, wherein the human subject is a patient having a gut microbiome devoid of any detectable levels of commensal bacteria.

104. The method of any one of claims 70-103 further comprising administering to the human subject antibiotics (e.g., broad spectrum antibiotics) or other standard-of-care treatment concurrent with the oligosaccharide composition.

105. The method of any one of claims 70-104, wherein the subject has been treated with antibiotics (e.g., broad spectrum antibiotics) or other standard-of-care treatment prior to administration with the oligosaccharide composition.

106. The method of any one of claims 70-105, wherein the oligosaccharide composition is administered to the subject one to twenty-eight days before a cancer treatment, surgery (e.g., transplant, e.g., hematopoietic stem cell), or admission to an intensive care unit

107. The method of any one of claims 70-106, wherein the oligosaccharide composition is administered to the subject one to twenty-eight days after a cancer treatment, surgery (e.g., transplant, e.g., hematopoietic stem cell), or admission to an intensive care unit.

108. The method of any one of claims 70-107, wherein the oligosaccharide composition is administered to the subject at least one to twenty-eight days after onset of a pathogen infection.

109. The method of any one of claims 70-108, wherein the method comprises administering the oligosaccharide composition to the intestines (e.g., the large intestine).

110. The method of any one of claims 70-109, wherein the oligosaccharide composition is self-administered to the subject.

111. The method of any one of claims 70-110, wherein the oligosaccharide composition is formulated as a pharmaceutical composition for oral delivery.

112. The method of any one of claims 70-111, wherein the oligosaccharide composition is orally administered to the subject.

113. The method of any one of claims 70-110, wherein the oligosaccharide composition is formulated as a pharmaceutical composition for delivery by a feeding tube.

114. The method of any one of claim 70-110 or 113, wherein the oligosaccharide composition is administered to the subject by a feeding tube.

115. The method of any one of claims 70-110, wherein the oligosaccharide composition is administered to the subject once per day or twice per day.
